(19) United States
(12) Patent Application Publication
(10) Pub. No.: US 2024/0182530 A1
(43) Pub. Date: Jun. 6, 2024

(54) POLYPEPTIDES THAT INTERACT WITH PEPTIDE TAGS AT LOOPS OR TERMINI AND USES THEREOF

(71) Applicant: Oxford University Innovation Limited, Oxford (GB)

(72) Inventors: Mark HOWARTH, Oxford (Oxfordshire) (GB); Vikash YADAV, Oxford (Oxfordshire) (GB); Matteo FERLA, Oxford (Oxfordshire) (GB)

(21) Appl. No.: 18/282,580

(22) PCT Filed: Apr. 1, 2022
(86) PCT No.: PCT/GB2022/050841

§ 371 Date: Sep. 18, 2023

(30) Foreign Application Priority Data
Apr. 8, 2021 (GB) 2104999.4

(51) Int. Cl. C07K 14/315; C07K 1/14; C12N 15/63

(52) U.S. Cl. CPC C07K 14/315; C07K 1/14; C12N 15/63; C07K 2319/90

(57) ABSTRACT

The present invention relates to a polypeptide that forms one part of a two-part linker in which the polypeptide spontaneously forms an isopeptide bond with a peptide tag, the second part of the two-part linker. Nucleic acid molecules encoding the polypeptide, vectors comprising said nucleic acid molecules, and host cells comprising said vectors and nucleic acid molecules are also provided. A kit comprising said two-part linker (i.e. peptide tag and polypeptide binding partner), and/or nucleic acid molecules/vectors is also provided. A method of producing the polypeptide and the uses of the polypeptide of the invention are also provided.

### FIELD OF THE INVENTION

The present invention relates in one aspect to a polypeptide that forms one part of a two-part linker in which the polypeptide (protein) spontaneously forms an isopeptide bond with a peptide tag, the second part of the two-part linker. In particular, the two-part linker may be viewed as a peptide tag and polypeptide binding partner cognate pair that can be conjugated via a covalent bond when contacted under conditions that allow the spontaneous formation of an isopeptide bond between the polypeptide of the invention and the peptide tag. In a second aspect, the invention also provides an affinity purification system comprising a modified polypeptide (protein) that binds selectively (e.g. specifically) and reversibly to its cognate peptide tag (ligand), i.e. does not spontaneously form an isopeptide bond with a peptide tag. Nucleic acid molecules encoding the polypeptides, vectors comprising said nucleic acid molecules, and host cells comprising said vectors and nucleic acid molecules are also provided. Kits comprising said polypeptides (e.g. peptide tag and polypeptide binding partner), and/or nucleic acid molecules/vectors are also provided. Further products comprising said polypeptides and uses of the polypeptides of the invention are also provided.

### BACKGROUND TO THE INVENTION

Cellular function depends on enormous numbers of reversible non-covalent protein-protein interactions and the precise arrangement of proteins in complexes influences and determines their function. Thus, the ability to engineer covalent protein-protein interactions can bring a range of new opportunities for basic research, synthetic biology and biotechnology. In particular, the conjugation of two or more proteins to form a so-called “fusion protein” can result in molecules with useful characteristics. For instance, clustering a single kind of protein often greatly enhances biological signals, e.g. the repeating antigen structures on vaccines. Clustering proteins with different activities can also result in complexes with improved activities, e.g. substrate channeling by enzymes.

Typically, covalent protein interactions are mediated through disulfide bonds, but disulfides are reversible, inapplicable in reducing cellular compartments, and can interfere with protein folding. Peptide tags are convenient tools for protein analysis and modification because their small size minimises the perturbation to protein function. Peptide tags are simple to genetically encode and their small size reduces disruption from (i) interfering with other interactions, (ii) cost of biosynthesis, and (iii) introduction of immunogenicity. However, interactions between peptide tags and their peptide or polypeptide binding partners are rarely of high affinity, which limits their utility in the formation of stable complexes.

Proteins that are capable of spontaneous isopeptide bond formation (so-called “isopeptide proteins”) have been advantageously used to develop peptide tag/polypeptide binding partner pairs (i.e. two-part linkers) which covalently bind to each other and provide irreversible interactions (see e.g. WO2011/098772, WO 2016/193746, WO 2018/197854 and WO2020/183198 all herein incorporated by reference). In this respect, proteins which are capable of spontaneous isopeptide bond formation may be expressed as separate fragments, to give a peptide tag and a polypeptide binding partner for the peptide tag, where the two fragments are capable of covalently reconstituting by isopeptide bond formation, thereby linking molecules or components fused to the peptide tag and its polypeptide binding partner.

Isopeptide bonds are amide bonds formed between carboxyl/carboxamide and amino groups, where at least one of the carboxyl or amino groups is outside of the protein main-chain (the backbone of the protein). Such bonds are chemically irreversible under typical biological conditions and they are resistant to most proteases. Since isopeptide bonds are covalent in nature, they result in the some of the strongest measured protein interactions. The isopeptide bond formed by a peptide tag and its polypeptide binding partner is stable under conditions where non-covalent interactions would rapidly dissociate, e.g. over long periods of time (e.g. weeks), at high temperature (to at least 95° C.), at high force, or with harsh chemical treatment (e.g. pH 2-11, organic solvent, detergents or denaturants).

In brief, a two-part linker, i.e. a peptide tag and its polypeptide binding partner (a so-called peptide tag/binding partner pair) may be derived from a protein capable of spontaneously forming an isopeptide bond (an isopeptide protein), wherein the domains of the protein are expressed separately to produce a peptide tag that comprises one of the residues involved in the isopeptide bond (e.g. an aspartate or asparagine) and a peptide or polypeptide binding partner (or “catcher”) that comprises the other residue involved in the isopeptide bond (e.g. a lysine) and at least one other residue required to form the isopeptide bond (e.g. a glutamate). Mixing the peptide tag and binding partner results in the spontaneous formation of an isopeptide bond between the tag and binding partner. Thus, by separately fusing the peptide tag and binding partner to different molecules or components, e.g. proteins, it is possible to covalently link said molecules or components together via an isopeptide bond formed between the peptide tag and binding partner, i.e. to form a linker between the molecules or components fused to the peptide tag and binding partner.

There are many efficient ways to connect proteins at their termini, from classic genetic fusion through to advanced enzymatic ligations and two-part peptide tag/polypeptide binding partner pairs (i.e. two-part linkers) such as those disclosed herein and referenced above. Extensive work has been done to establish post-translational connection of protein units, including native chemical ligation, split inteins, sortase and butelase. However, several of these methods of connection are inappropriate for ligating proteins at internal sites. For example, split inteins must be at the termini of proteins. Similarly, sortase enzymes are almost always used at the termini of proteins, and require very high concentrations of the oligoglycine reactant. There has been much less attention to protein-protein ligation at internal sites, where there is more steric hindrance and fewer accessible chemistries than at termini. N- and C-termini of natural proteins are often highly flexible and more exposed, facilitating reaction, whereas internal loops may adopt diverse structures and there are countless examples of insertion of a peptide tag in a loop interfering with protein folding or function. It is therefore much more difficult to connect proteins together at internal sites, such as protein loops because of the lower flexibility and more variable environment.

However, in some applications, it is necessary or desirable to connect proteins together at internal sites. Numerous proteins are not amenable to fusions at their termini, including those with termini which are key for the function of the protein (e.g. the proteasome), or those with termini which are located on the intracellular side of the plasma membrane (e.g. tetraspanins and many ion channels), or buried at inter-protein interfaces (e.g. Qβ virus-like particles). Even when termini are a possible fusion site, internal fusion, such as loop fusion, may still be preferred to control protein orientation, such as at the surface of diagnostics, in a multi-enzyme complex, or in a vaccine conjugate.

The present inventors previously developed another two-part peptide tag/polypeptide binding partner system, known as SpyTag/SpyCatcher, based on the CnaB2 domain of the Streptococcus pyogenes FbaB protein (Zakeri et al., 2012, Proc Natl Acad Sci USA 109, E690-E697). The most recent iteration of this system, SpyTag003/SpyCatcher003, is the pair previously established to have the fastest reactivity for protein-protein reaction at termini (see WO2020/183198 incorporated herein by reference). However, as shown in the Examples, although SpyTag003 could be inserted internally into specific loop regions of certain proteins and could react with its cognate partner SpyCatcher003, the rate of reaction was significantly reduced as compared to when SpyTag003 was fused at a terminus of the same protein. Moreover, in certain cases, when SpyTag003 was inserted into a loop region of a given protein, expression of the protein was not possible at all.

An alternative system for joining proteins together at internal sites may be provided by expressing the domains of an isopeptide protein which comprise the residues involved in isopeptide bond formation separately, i.e. as three separate fragments, i.e. two peptides and a polypeptide (see e.g. Fierer et al. 2014, PNAS E1176-E1181). One such system was developed by the present inventors based on RrgA (see WO2018/189517 incorporated herein by reference). The RrgA protein was split into three separate components; a first peptide tag (termed SnoopTagJr) which comprises one of the residues involved in the isopeptide bond (e.g. a lysine), a second peptide tag (termed DogTag) which comprises the other residue involved in the isopeptide bond (e.g. an asparagine) and a polypeptide (termed SnoopLigase) which comprises the residue involved in mediating the isopeptide bond formation (e.g. a glutamate). Mixing all three fragments, i.e. both peptides and the polypeptide, results in the formation of an isopeptide bond between the two peptides comprising the residues that react to form the isopeptide bond, i.e. between SnoopTagJr and DogTag. However, the reaction rate of SnoopLigase is relatively slow (˜48 h to reach completion) which limits its application, especially in cellular systems. In addition, the SnoopLigase system is not compatible with certain buffers, and requires relatively high concentrations of the constituent components, which is not always possible in practice, particularly with mammalian expression systems, for example.

Accordingly, there is a need for an improved linker system which is capable of joining proteins at internal sites.

A peptide tag/binding partner pair (two-part linker), termed RrgATag/RrgACatcher, has been derived from the adhesin protein RrgA from Streptococcus pneumoniae, a Gram-positive bacterium which can cause septicaemia, pneumonia and meningitis in humans. A spontaneous isopeptide bond forms in the D4 immunoglobulin-like domain of RrgA between residues Lys742 and Asn854. This D4 domain was previously split into a pair of linkers termed RrgATag (SEQ ID NO: 4) and RrgACatcher (SEQ ID NO: 6) (see WO 2016/193746 which is incorporated herein by reference). RrgATag is derived from residues 838-856 of the RrgA protein, and thus includes the Asn854 residue, whilst RrgACatcher (also known as R2Catcher) corresponds to residues 734-837 of the RrgA protein, and thus includes the Lys742 residue. Accordingly, RrgATag (SEQ ID NO: 4) and RrgACatcher (SEQ ID NO: 6) are capable of spontaneously forming an isopeptide bond.

Although purified RrgATag and RrgACatcher could successfully reconstitute and react upon mixing, the rate of isopeptide bond formation was relatively slow, particularly when the linkers were present at concentrations equivalent to cellular expression levels. An engineered version of RrgATag (DogTag, SEQ ID NO: 3) was shown to have faster reconstitution, i.e. a faster rate of formation of the isopeptide bond with RrgACatcher. RrgATag contains a Thr residue instead of a Gly residue at the position corresponding to position 842 of RrgA. This sequence was further modified to extend the peptide to contain residues corresponding to residues 857-860 of RrgA and the Asp residue corresponding to position 848 within RrgATag was substituted with Gly. RrgATag with these two modifications (C-terminal extension and D848G) was referred to as RrgATag2 (SEQ ID NO: 5). In addition, the Asn residue corresponding to position 847 of RrgA within RrgATag and RrgATag2 was substituted with Asp. RrgATag with all three of these modifications (C-terminal extension, N847D, and D848G) was referred to as DogTag (SEQ ID NO: 3).

DogTag had an improved reaction rate with RrgACatcher, relative to RrgATag and other versions of the tag, e.g. R2Tag (SEQ ID NO: 17) as shown in the Examples, but the reaction rate was still slow at low concentrations. Moreover, the RrgACatcher polypeptide was observed to have limited solubility in certain conditions.

### SUMMARY OF THE INVENTION

The present inventors have now surprisingly determined that the reaction rate of the RrgACatcher polypeptide can be increased by at least an order of magnitude, and that the solubility of the polypeptide in common buffers and conditions can be significantly improved, by modifying (i.e. mutating) the amino acid sequence of the RrgACatcher polypeptide. Notably and unexpectedly, the modifications that result in the increased reaction rate and solubility do not adversely affect other desirable properties of the polypeptide. Thus, the modified RrgACatcher polypeptide of the invention (termed DogCatcher, SEQ ID NO: 1) has a reaction rate with DogTag which is more than an order of magnitude greater than the original RrgACatcher polypeptide, and can be used in various applications under a wide range of conditions because of its increased solubility.

Whilst not wishing to be bound by theory, it is hypothesised that of the ten modifications to the RrgACatcher polypeptide that result in the DogCatcher polypeptide, seven of these (termed the “solubility modifications”) may function independently to increase the solubility of the polypeptide. The sequence of RrgACatcher containing all seven of the solubility modifications is termed RrgACatcherB or R2CatcherB (SEQ ID NO: 8). It is thought that the remaining three modifications which distinguish DogCatcher from RrgACatcher (termed the “reactivity modifications”) may function independently to increase the rate of reaction with the DogTag peptide. The sequence of RrgACatcher containing all three of the reactivity modifications is provided in SEQ ID NO: 9. Thus, it is contemplated that each of the solubility and reactivity modifications in the polypeptide of the invention (DogCatcher (SEQ ID NO: 1) or DogCatcher variant (e.g. SEQ ID NO: 8 or 9)) relative to the amino acid sequence of RrgACatcher may separately improve the solubility and reactivity of the polypeptide, respectively.

It is further contemplated that the polypeptide exemplified herein (i.e. DogCatcher, SEQ ID NO: 1) may be truncated at the N-terminus and/or at the C-terminus without significantly reducing the activity of the polypeptide. In particular, SEQ ID NO: 1 may be truncated by up to 4 amino acids at the N-terminus (e.g. 1, 2, 3, or 4 amino acids) and/or by up to 5 amino acids at the C-terminus (e.g. 1, 2, 3, 4 or 5 amino acids).

Advantageously, the polypeptide (mutant “catcher” or peptide tag binding partner) of the invention (DogCatcher, SEQ ID NO: 1) may thus be used with its cognate peptide tag, e.g. DogTag (SEQ ID NO: 3), (i.e. as a two-part linker) in utilities where only low concentrations of the peptide tag and polypeptide binding partner are available, e.g. in vivo. The polypeptide (peptide tag binding partner) of the invention also may be particularly useful in analytical assays that require high sensitivity and/or speed, e.g. Western blots in which the peptide tag (e.g. DogTag, SEQ ID NO: 3) is being used as an epitope tag. The improved rate constant of the mutant catcher (polypeptide) of the invention is also advantageous in reactions in which the tag and/or catcher are fused to molecules or components that may slow the reaction (e.g. large proteins) and in reactions where molecules or components fused to the tag and/or catcher cause steric hindrance, such as in the formation of virus-like particles for vaccine assembly.

In this regard, the present inventors noted that the sequence of Domain 4 of RrgA from which DogTag is derived (residues 838-860) forms a β-hairpin which comprises the reactive Asn854 residue, and thus hypothesised that this could form the foundation of a loop-friendly Tag/Catcher pair (i.e. a Tag/Catcher pair capable of joining proteins at internal sites, such as protein loops). It was surprisingly and advantageously found that DogTag could be inserted into a range of loop sites in different proteins without disrupting the expression or function of said proteins, and that the rate of reaction between the polypeptide of the present invention (DogCatcher, SEQ ID NO: 1) and its binding partner (DogTag, SEQ ID NO: 3) was comparable regardless of whether DogTag was inserted into a protein at a terminal site or an internal, e.g. loop, site. The DogTag/DogCatcher two-part linker, involving the polypeptide of the present invention, exhibited a rate of reaction approximately 10-fold faster than that of SpyTag003/SpyCatcher003 when the peptide tags were inserted into certain protein loop sites. As is set out in more detail in the Examples below, the DogTag/DogCatcher two-part linker was demonstrated to be functional when DogTag was inserted internally in proteins that are predominantly α-helical, predominantly β-sheet, or α+β folds. Moreover, as a result of the aforementioned mutations, the DogCatcher polypeptide is soluble in a range of different buffers, and thus the DogTag/DogCatcher two-part linker can be used at lower concentrations in a variety of conditions.

Thus, the polypeptide of the present invention (DogCatcher, SEQ ID NO: 1) forms one part of a two-part linker system which is capable of spontaneously forming an isopeptide bond at a high reaction rate where the peptide tag is inserted internally within molecules or components of interest, for example where it is inserted at internal protein, e.g. loop, sites. This two-part linker therefore provides an improved method of introducing covalent protein ligation between certain proteins, particularly where the termini of at least one of the proteins are not optimal sites for fusion.

Alternatively viewed, the polypeptide of the invention (DogCatcher, SEQ ID NO: 1), in combination with its cognate peptide tag (DogTag, SEQ ID NO: 3), provides a two-part linker system which is particularly useful for connecting proteins together at internal sites, such as protein loops.

Accordingly, in one aspect, the present invention provides a polypeptide (peptide tag binding partner) comprising:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70 and one or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein if the amino acid sequence comprises proline at position 75, it also comprises one or more amino acid residues selected from 1)-6) and 8)-10), and wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66 and one or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein if the amino acid sequence comprises proline at position 71, it also comprises one or more amino acid residues selected from 1)-5) and 7)-9), and wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In an alternative embodiment, the polypeptide of the present invention may comprise:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70, one or more of the following:
1) glutamic acid at position 4;
2) aspartic acid at position 11;
3) threonine at position 13;
4) aspartic acid at position 47;
5) threonine at position 59;
6) proline at position 75; and
7) aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66, one or more of the following:
1) aspartic acid at position 7;
2) threonine at position 9;
3) aspartic acid at position 43;
4) threonine at position 55;
5) proline at position 71; and
6) aspartic acid at position 88;
and one or more of the following:
1) isoleucine at position 65;
2) serine at position 83; and
3) arginine at position 85;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

Alternatively viewed, the invention provides a polypeptide (peptide tag binding partner) comprising:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70 and one or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein: (A) if the amino acid sequence comprises proline at position 75, it also comprises one or more amino acid residues selected from 1)-6) and 8)-10); or (B) the amino acid sequence comprises at least one amino acid residue selected from 1)-5), 7) and 10) and one amino acid residue selected from 6), 8) and 9),
and wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66 and one or more of the following:
1) aspartic acid at position 7;
2) threonine at position 9;
3) aspartic acid at position 43;
4) threonine at position 55;
5) isoleucine at position 65;
6) proline at position 71;
7 serine at position 83;
8) arginine at position 85; and
9) aspartic acid at position 88;
wherein: (A) if the amino acid sequence comprises proline at position 71, it also comprises one or more amino acid residues selected from 1)-5) and 7)-9); or (B) the amino acid sequence comprises at least one amino acid residue selected from 1)-4), 6) and 9) and 1 amino acid residue selected from 5), 7) and 8),
and wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In another aspect, the invention provides a recombinant or synthetic polypeptide comprising a peptide or polypeptide linked to the polypeptide of the invention.

In another aspect, the invention provides the use of a polypeptide of the invention to conjugate two molecules or components via an isopeptide bond,
wherein said molecules or components conjugated via an isopeptide bond comprise:
a) a first molecule or component comprising a polypeptide of the invention; and
b) a second molecule or component comprising a peptide selected from:
(i) a peptide comprising an amino acid sequence as set forth in any one of SEQ ID NOs: 3-5 or 17; and
(ii) a peptide comprising an amino acid sequence with at least 80% sequence identity to a sequence as set forth in any one of SEQ ID NOs: 3-5 or 17 (e.g. at least 85, 90 or 95% identical to a sequence as set forth in any one of SEQ ID NOs: 3-5 or 17) wherein the amino acid sequence comprises an asparagine residue at position 17 and optionally comprises a threonine residue at position 5, an aspartic acid residue at position 10 and a glycine residue at position 11,
and wherein said peptide is capable of spontaneously forming an isopeptide bond with a polypeptide comprising an amino acid sequence as set forth in SEQ ID NO: 1, wherein said isopeptide bond forms between the asparagine residue at position 17 of SEQ ID NO: 3, 4, 5 or 17 and the lysine residue at position 9 of SEQ ID NO: 1.

Alternatively viewed, the invention provides a process for conjugating two molecules or components via an isopeptide bond comprising:
a) providing a first molecule or component comprising a polypeptide of the invention;
b) providing a second molecule or component comprising a peptide selected from:
(i) a peptide comprising an amino acid sequence as set forth in any one of SEQ ID NOs: 3-5 or 17; and
(ii) a peptide comprising an amino acid sequence with at least 80% sequence identity to a sequence as set forth in any one of SEQ ID NOs: 3-5 or 17 (e.g. at least 85, 90 or 95% identical to a sequence as set forth in any one of SEQ ID NOs: 3-5 or 17) wherein the amino acid sequence comprises an asparagine residue at position 17 and optionally comprises a threonine residue at position 5, an aspartic acid residue at position 10 and a glycine residue at position 11,
wherein said peptide is capable of spontaneously forming an isopeptide bond with a polypeptide comprising an amino acid sequence as set forth in SEQ ID NO: 1, wherein said isopeptide bond forms between the asparagine residue at position 17 of SEQ ID NO: 3, 4, 5 or 17 and the lysine residue at position 9 of SEQ ID NO: 1; and
c) contacting said first and second molecules or components under conditions that enable the spontaneous formation of an isopeptide bond between the polypeptide and peptide, thereby conjugating said first molecule or component to said second molecule or component via an isopeptide bond to form a complex.

In yet another aspect, the invention provides a kit, preferably for use in the use or process of the invention, wherein said kit comprises:
(a) a polypeptide of the invention, optionally conjugated or fused to a molecule or component; and
(b) a peptide, optionally conjugated or fused to a molecule or component, wherein the peptide is selected from:
(i) a peptide comprising an amino acid sequence as set forth in any one of SEQ ID NOs: 3-5 or 17; and
(ii) a peptide comprising an amino acid sequence with at least 80% sequence identity to a sequence as set forth in any one of SEQ ID NOs: 3-5 or 17 (e.g. at least 85, 90 or 95% identical to a sequence as set forth in any one of SEQ ID NOs: 3-5) wherein the amino acid sequence comprises an asparagine residue at position 17 and optionally comprises a threonine residue at position 5, an aspartic acid residue at position 10 and a glycine residue at position 11,
wherein said peptide is capable of spontaneously forming an isopeptide bond with a polypeptide comprising an amino acid sequence as set forth in SEQ ID NO: 1, wherein said isopeptide bond forms between the asparagine residue at position 17 of SEQ ID NO: 3, 4, 5 or 17 and the lysine residue at position 9 of SEQ ID NO: 1; and/or
(c) a nucleic acid molecule, particularly a vector, encoding a polypeptide as defined in (a); and/or
(d) a nucleic acid molecule, particularly a vector, encoding a peptide as defined in (b).

The inventors have previously determined that “Catcher” polypeptides may be modified to establish an affinity purification system for their cognate peptide tag (see e.g. WO 2020/115252, which is incorporated herein by reference). The system therefore may be viewed as a two-part system comprising a polypeptide (an affinity purification polypeptide) and its cognate peptide tag (affinity tag) that are capable of forming a stable and reversible non-covalent complex (i.e. a polypeptide:ligand complex) that can be dissociated under appropriate conditions to facilitate the isolation and/or purification of a molecule or component (fusion partner) conjugated or fused to said peptide tag.

Upon determining that the properties of the RrgACatcher polypeptide could be improved by introducing mutations found in DogCatcher, the inventors identified the possibility of modifying the polypeptide defined above to establish a DogTag affinity purification system. While not wishing to be bound by theory, it is thought that mutation of the DogCatcher polypeptide at the position of the activating glutamic acid residue in the D4 domain of RrgA (803E) is sufficient to abrogate the formation of an isopeptide bond between DogCatcher and DogTag, whilst maintaining a selective, stable and reversible non-covalent interaction with DogTag.

Accordingly, in a further aspect the invention provides a polypeptide (an affinity purification polypeptide) comprising:
i) an amino acid sequence as set forth in SEQ ID NO: 18, wherein X at position 70 is not glutamic acid or aspartic acid (i.e. X at position 70 may be any amino acid other than glutamic acid or aspartic acid), optionally wherein X at position 70 is selected from alanine, glycine, serine, asparagine, or threonine;
ii) a portion of (i) comprising an amino acid sequence as set forth in SEQ ID NO: 19, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 18, wherein X at position 70 is not glutamic acid or aspartic acid, optionally wherein X at position 70 is selected from alanine, glycine, serine, asparagine, or threonine and wherein the amino acid sequence comprises one or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein if the amino acid sequence comprises proline at position 75, it also comprises one or more amino acid residues selected from 1)-6) and 8)-10), and wherein the specified amino acid residues are at positions equivalent to the positions in SEQ ID NO: 18; or
iv) a portion of (iii) comprising an amino acid sequence with at least 80% sequence identity to a sequence as set forth in SEQ ID NO: 19, wherein X at position 66 is not glutamic acid or aspartic acid, optionally wherein X at position 66 is selected from alanine, glycine, serine, asparagine, or threonine and wherein the amino acid sequence comprises one or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein if the amino acid sequence comprises proline at position 71, it also comprises one or more amino acid residues selected from 1)-5) and 7)-9), and wherein the specified amino acid residues are at positions equivalent to the positions in SEQ ID NO: 19,
and wherein the polypeptide binds selectively and reversibly to a peptide comprising an amino acid sequence as set forth in SEQ ID NO: 3.

In an alternative aspect, the invention provides a polypeptide (an affinity purification polypeptide) comprising:
i) an amino acid sequence as set forth in SEQ ID NO: 18, wherein X at position 70 is not glutamic acid or aspartic acid, optionally wherein X at position 70 is selected from alanine, glycine, serine, asparagine, or threonine;
ii) a portion of (i) comprising an amino acid sequence as set forth in SEQ ID NO: 19, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 18, wherein X at position 70 is not glutamic acid or aspartic acid, optionally wherein X at position 70 is selected from alanine, glycine, serine, asparagine, or threonine and wherein the amino acid sequence comprises one or more of the following:
1) glutamic acid at position 4;
2) aspartic acid at position 11;
3) threonine at position 13;
4) aspartic acid at position 47;
5) threonine at position 59;
6) proline at position 75; and
7 aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 19, wherein X at position 66 is not glutamic acid or aspartic acid, optionally wherein X at position 66 is selected from alanine, glycine, serine, asparagine, or threonine and wherein the amino acid sequence comprises one or more of the following:
1) aspartic acid at position 7;
2) threonine at position 9;
3) aspartic acid at position 43;
4) threonine at position 55;
5) proline at position 71; and
6) aspartic acid at position 88;
and one or more of the following:
1) isoleucine at position 65;
2) serine at position 83;
3) arginine at position 85;
wherein the specified amino acid residues are at positions equivalent to the positions in SEQ ID NO: 19,
and wherein the polypeptide binds selectively and reversibly to a peptide comprising an amino acid sequence as set forth in SEQ ID NO: 3.

Alternatively viewed, the invention provides a polypeptide (an affinity purification polypeptide) comprising:
i) an amino acid sequence as set forth in SEQ ID NO: 18, wherein X at position 70 is not glutamic acid or aspartic acid (i.e. X at position 70 may be any amino acid other than glutamic acid or aspartic acid), optionally wherein X at position 70 is selected from alanine, glycine, serine, asparagine, or threonine;
ii) a portion of (i) comprising an amino acid sequence as set forth in SEQ ID NO: 19, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 18, wherein X at position 70 is not glutamic acid or aspartic acid, optionally wherein X at position 70 is selected from alanine, glycine, serine, asparagine, or threonine and wherein the amino acid sequence comprises one or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein: (A) if the amino acid sequence comprises proline at position 75, it also comprises one or more amino acid residues selected from 1)-6) and 8)-10); or (B) the amino acid sequence comprises at least one amino acid residue selected from 1)-5), 7) and 10) and one amino acid residue selected from 6), 8) and 9),
and wherein the specified amino acid residues are at positions equivalent to the positions in SEQ ID NO: 18; or
iv) a portion of (iii) comprising an amino acid sequence with at least 80% sequence identity to a sequence as set forth in SEQ ID NO: 19, wherein X at position 66 is not glutamic acid or aspartic acid, optionally wherein X at position 66 is selected from alanine, glycine, serine, asparagine, or threonine and wherein the amino acid sequence comprises one or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein: (A) if the amino acid sequence comprises proline at position 71, it also comprises one or more amino acid residues selected from 1)-5) and 7)-9); or (B) the amino acid sequence comprises at least one amino acid residue selected from 1)-4), 6) and 9) and 1 amino acid residue selected from 5), 7) and 8),
and wherein the specified amino acid residues are at positions equivalent to the positions in SEQ ID NO: 19,
and wherein the polypeptide binds selectively and reversibly to a peptide comprising an amino acid sequence as set forth in SEQ ID NO: 3.

In a further aspect, the invention provides a process for purifying or isolating a molecule or component comprising a peptide having an amino acid sequence with at least 80% sequence identity to a sequence as set forth in one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11, said process comprising:
a) providing a solid substrate on which a polypeptide (affinity purification polypeptide) as defined above is immobilised;
b) providing a sample comprising said molecule or component;
c) contacting the solid substrate of a) with the sample of b) under conditions that enable said peptide to selectively bind to said polypeptide, thereby forming a non-covalent complex between said polypeptide immobilised on the solid substrate and molecule or component comprising said peptide;
d) washing the solid substrate with a buffer;
e) separating the molecule or component comprising the peptide from the polypeptide immobilised on the solid substrate.

In yet another embodiment, the invention provides the use of a polypeptide (affinity purification polypeptide) as defined above to purify or isolate a molecule or component comprising a peptide having an amino acid sequence with at least 80% sequence identity to a sequence as set forth in one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11.

In another aspect, the invention provides an apparatus for use in the process or use defined above comprising a solid substrate on which a polypeptide (affinity purification polypeptide) as defined above is immobilised.

In a further aspect, the invention provides a kit for use in preparing a solid substrate on which a polypeptide (affinity purification polypeptide) as defined above is immobilised, comprising:
a) a polypeptide (affinity purification polypeptide) as defined above; and
b) means for immobilising the polypeptide of a) on a solid substrate.

In a further aspect, the invention provides a nucleic acid molecule comprising a nucleotide sequence which encodes a polypeptide of the invention or a recombinant or synthetic polypeptide of the invention defined above.

In still another aspect, the invention provides a vector comprising the nucleic acid molecule of the invention.

In another aspect, the invention provides a cell comprising the nucleic acid molecule or vector of the invention.

The invention also provides a process for producing or expressing the polypeptide or recombinant polypeptide of the invention comprising the steps of:
a) transforming or transfecting a host cell with a vector of the invention;
b) culturing the host cell under conditions which allow the expression of the polypeptide; and optionally
c) isolating the polypeptide.

### DETAILED DESCRIPTION

As discussed above, it is contemplated that each of the solubility and reactivity mutations in DogCatcher relative to RrgACatcher may separately and independently improve the solubility and reactivity of the polypeptide, respectively. Accordingly, in some embodiments, the polypeptide comprises:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70 and two or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66 and two or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In some embodiments where the polypeptide comprises two or more residues selected from 1)-10) above, the polypeptide comprises at least one of the solubility modifications (i.e. at least one of 1)-5), 7) or 10)) and at least one of the reactivity modifications (i.e. at least one of 6), 8) or 9)).

Based on the data in Table 1, and without wishing to be bound by theory, it is hypothesised that the presence of a proline residue at a position equivalent to position 75 of SEQ ID NO: 1 (equivalent to position 71 of SEQ ID NO: 2) has a particularly beneficial effect on the solubility of the polypeptide of the invention. In some embodiments, the polypeptide comprises a proline residue at a position equivalent to position 75 of SEQ ID NO: 1 (equivalent to position 71 of SEQ ID NO: 2). Accordingly, the two or more amino acids may be the proline at position 75 of SEQ ID NO: 1 and any one or more of 1)-6) and 8)-10) or the proline at position 71 of SEQ ID NO: 2, and any one or more of 2)-6) and 8)-10) (or 1)-5) and 7)-9) using the numbering in part (iv) above). However, any combination of two or more amino acids from those listed above are contemplated herein. In some embodiments, the two or more amino acids may be the proline at position 75 of SEQ ID NO: 1 and any one or more of 1), 4) and 10) or the proline at position 71 of SEQ ID NO: 2, and one or both of 4) or 10) (or 3) or 9) using the numbering in part (iv) above).

In a further embodiment of the invention, the polypeptide comprises:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70 and three or more, four or more, five or more, six or more, seven or more, eight or more, or nine or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66 and three or more, four or more, five or more, six or more, seven or more or eight or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In some embodiments, the four or more residues include residues 1), 4), 7) and 10) and optionally one, two or three of 6), 8) and 9) (using the numbering in part (iii) above).

As noted above, of the ten modifications that were made to the RrgACatcher polypeptide (SEQ ID NO: 6) that result in the DogCatcher polypeptide (SEQ ID NO: 1), seven of these (termed the “solubility modifications”) are suggested to function to increase the solubility of the polypeptide, and three of these (termed the “reactivity modifications”) are suggested to function to increase the rate of reaction with the DogTag peptide.

The seven solubility modifications are, in terms of the residues in the original RrgA protein: D737E, N774D, N746T, N780D, K792T, A808P, and N825D. In terms of the residues in SEQ ID NO: 1, these solubility modifications correspond to:
1) glutamic acid at position 4;
2) aspartic acid at position 11;
3) threonine at position 13;
4) aspartic acid at position 47;
5) threonine at position 59;
6) proline at position 75; and
7) aspartic acid at position 92.

As noted above, the sequence of RrgACatcher containing all seven of the solubility modifications is termed RrgACatcherB or R2CatcherB (SEQ ID NO: 8). In some embodiments, the polypeptide of the present invention may comprise all of the solubility modifications, i.e. may comprise an amino acid sequence as set forth in SEQ ID NO: 8 or a variant amino acid sequence thereof that results in a functional polypeptide as defined herein, e.g. that is capable of spontaneously forming an isopeptide bond with a peptide comprising an amino acid sequence as set forth in SEQ ID NO: 3. Alternatively put, the polypeptide of the present invention may comprise:
i) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70 and all of the following:
1) glutamic acid at position 4;
2) aspartic acid at position 11;
3) threonine at position 13;
4) aspartic acid at position 47;
5) threonine at position 59;
6) proline at position 75; and
7) aspartic acid at position 92
wherein the specified amino acid residues are at positions equivalent to the positions in SEQ ID NO: 1; or
ii) a portion of (i) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66 and all of the following:
1) aspartic acid at position 7;
2) threonine at position 9;
3) aspartic acid at position 43;
4) threonine at position 55;
5) proline at position 71; and
6) aspartic acid at position 88;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

The three reactivity modifications are, in terms of the residues in the original RrgA protein: F802I, A820S, and Q822R. In terms of the residues in SEQ ID NO: 1, these solubility modifications correspond to:
1) isoleucine at position 69;
2) serine at position 87; and
3) arginine at position 89.

As noted above, the sequence of RrgACatcher containing all three of the reactivity modifications is provided in SEQ ID NO: 9. In some embodiments, the polypeptide of the present invention may comprise all of the reactivity modifications, i.e. may comprise an amino acid sequence as set forth in SEQ ID NO: 9, or a variant amino acid sequence thereof that results in a functional polypeptide as defined herein, e.g. that is capable of spontaneously forming an isopeptide bond with a peptide comprising an amino acid sequence as set forth in SEQ ID NO: 3 under suitable conditions. Alternatively put, the polypeptide of the present invention may comprise:
i) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70 and all of the following:
1) isoleucine at position 69;
2) serine at position 87; and
3) arginine at position 89;
wherein the specified amino acid residues are at positions equivalent to the positions in SEQ ID NO: 1; or
ii) a portion of (i) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66 and all of the following:
1) isoleucine at position 65;
2) serine at position 83; and
3) arginine at position 85;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In some embodiments, the polypeptide of the present invention comprises a proline residue at a position equivalent to position 75 of SEQ ID NO: 1 (equivalent to position 71 of SEQ ID NO: 2). Accordingly, the polypeptide of the present invention may comprise a proline residue at said position, and one or more of the reactivity modifications. In this regard, the polypeptide of the present invention may comprise:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70, a proline at position 75 and one or more of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66, a proline at position 71 and one or more of the following:
1) isoleucine at position 65;
2) serine at position 83; and
3) arginine at position 85;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In a further embodiment, the polypeptide of the present invention comprises a proline residue at a position equivalent to position 75 in SEQ ID NO: 1, one or more additional solubility modifications, and one of more reactivity modifications. That is to say, the polypeptide of the present invention may comprise:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70, a proline at position 75, one or more of the following:
1) glutamic acid at position 4;
2) aspartic acid at position 11;
3) threonine at position 13;
4) aspartic acid at position 47;
5) threonine at position 59; and
6) aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66, a proline at position 71, one or more of the following:
1) aspartic acid at position 7;
2) threonine at position 9;
3) aspartic acid at position 43;
4) threonine at position 55; and
5) aspartic acid at position 88;
and one or more of the following:
1) isoleucine at position 65;
2) serine at position 83; and
3) arginine at position 85;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In a further embodiment, the polypeptide of the present invention comprises:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70, and two or more, three or more, four or more, five or more or six or more of the following:
1) glutamic acid at position 4;
2) aspartic acid at position 11;
3) threonine at position 13;
4) aspartic acid at position 47;
5) threonine at position 59;
6) proline at position 75; and
7) aspartic acid at position 92;
and one or more (e.g. two or three) of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66, two or more, three or more, four or more or five or more of the following:
1) aspartic acid at position 7;
2) threonine at position 9;
3) aspartic acid at position 43;
4) threonine at position 55;
5) proline at position 71; and
6) aspartic acid at position 88;
and one or more (e.g. two or three) of the following:
1) isoleucine at position 65;
2) serine at position 83; and
3) arginine at position 85;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In some embodiments, the two or more amino acids selected from the list of solubility modifications may be the proline at position 75 of SEQ ID NO: 1 and any one or more of 1)-5) and 7) (using the numbering part (iii) above) or the proline at position 71 of SEQ ID NO: 2, and any one of 1)-4) and 6) (using the numbering part (iii) above). However, any combination of two or more amino acids from those listed above is contemplated herein. In some embodiments, the polypeptide comprises at least glutamic acid at position 4; aspartic acid at position 47; proline at position 75; and aspartic acid at position 92. In some embodiments, the truncated polypeptide (polypeptide portion) comprises at least aspartic acid at position 43; proline at position 71; and aspartic acid at position 88.

In some embodiments, the polypeptide of the invention comprises all of the solubility modifications and one or more, e.g. all, of the reactivity modifications. Accordingly, the polypeptide of the present invention may comprise:
i) an amino acid sequence as set forth in SEQ ID NO: 1; or
ii) a portion of (i) comprising an amino acid sequence as set forth in SEQ ID NO: 2;
iii) an amino acid sequence with at least 80% sequence identity to a sequence as set forth in SEQ ID NO: 1 (e.g. at least 85, 90, 95, 96, 97, 98 or 99% identical to a sequence as set forth in SEQ ID NO: 1), wherein said amino acid sequence comprises a lysine at position 9, a glutamic acid at position 70 and all of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2 (e.g. at least 85, 90, 95, 96, 97, 98 or 99% identical to a sequence as set forth in SEQ ID NO: 2), wherein the amino acid sequence comprises a lysine at position 5, a glutamic acid at position 66 and all of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

In a particularly preferred embodiment, all ten of the amino acids mentioned above in relation to SEQ ID NO: 1 (nine in relation to SEQ ID NO: 2) which distinguish DogCatcher from RrgACatcher are present in the variant polypeptide of the invention. In embodiments in which the polypeptide variants (i.e. sequence identity related polypeptides and portions thereof) of the invention do not contain all of the residues specified above, it is typically preferred that in the specified positions the variants contain the amino acid residues at the equivalent positions in the RrgACatcher polypeptide (SEQ ID NO: 6) or conservative substitutions thereof. The equivalent positions can readily be determined by comparing the amino acid sequence of the polypeptide variant with SEQ ID NO: 6, e.g. using the BLASTP algorithm.

Thus, by way of example, in embodiments where the polypeptide of the invention comprises an amino acid sequence with at least 80% sequence identity to a sequence defined herein (e.g. as set forth in SEQ ID NO: 1 or 18), if the residue at position 4 (or the equivalent position) is not glutamic acid, it is preferred that the residue is aspartic acid. Similarly, if the residue at position 11 (or the equivalent position) is not aspartic acid, it is preferred that the residue is asparagine. If the residue at position 13 (or the equivalent position) is not threonine, it is preferred that the residue is asparagine. If the residue at position 47 (or the equivalent position) is not aspartic acid, it is preferred that the residue is asparagine. If the residue at position 59 (or the equivalent position) is not threonine, it is preferred that the residue is lysine. If the residue at position 69 (or the equivalent position) is not isoleucine, it is preferred that the residue is phenylalanine. If the residue at position 75 (or the equivalent position) is not proline, it is preferred that the residue is alanine. If the residue at position 87 (or the equivalent position) is not serine, it is preferred that the residue is alanine. If the residue at position 89 (or the equivalent position) is not arginine, it is preferred that the residue is glutamine. If the residue at position 92 (or the equivalent position) is not aspartic acid, it is preferred that the residue is asparagine. This applies to other residues specified below.

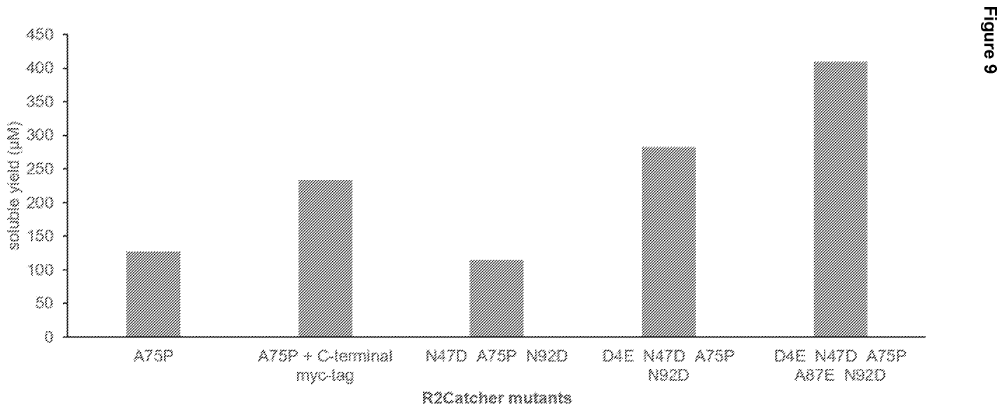

However, in some embodiments, if the residue at position 87 (or the equivalent position) is not serine, it is preferred that the residue is glutamic acid. As shown in FIG. 9 below, a glutamic acid residue at this position may further improve the solubility of the polypeptide. Thus, in some embodiments, the polypeptide comprises only two of the “reactivity” modifications, isoleucine at position 69 (or the equivalent position) and arginine at position 89 (or the equivalent position) and comprises a glutamic acid at position 87 (or the equivalent position).

In some embodiments, a polypeptide variant of the present invention may differ from SEQ ID NO: 1 or 18 by, for example, 1 to 30, 1 to 25, 1 to 20, 1 to 15, 1 to 10, 1 to 8, 1 to 6, 1 to 5, 1 to 4, e.g. 1, 2 or 3 amino acid substitutions, insertions and/or deletions, preferably 1 to 21, 1 to 20, 1 to 15, 1 to 10, 1 to 8, 1 to 6, 1 to 5, 1 to 4, e.g. 1, 2 to 3 amino acid substitutions and/or 1 to 15, 1 to 10, 1 to 9, 1 to 8, 1 to 6, 1 to 5, 1 to 4, e.g. 1, 2 or 3 amino acid deletions. As discussed below, in some embodiments, it is preferred that deletions are at the N- and/or C-terminus, i.e. truncations, thereby generating polypeptide portions of SEQ ID NO: 1 as defined above, e.g. SEQ ID NO: 2 or 19.

In some embodiments, any mutations that are present in the polypeptide of the present invention relative to the exemplified polypeptides (e.g. SEQ ID NOs: 1 or 18) may be conservative amino acid substitutions. A conservative amino acid substitution refers to the replacement of an amino acid by another which preserves the physicochemical character of the polypeptide (e.g. D may be replaced by E or vice versa, N by Q, or L or I by V or vice versa). Thus, generally the substituting amino acid has similar properties, e.g. hydrophobicity, hydrophilicity, electronegativity, bulky side chains etc. to the amino acid being replaced. Isomers of the native L-amino acid e.g. D-amino acids may be incorporated.

Thus, in some embodiments in which the polypeptide variants of the invention do not contain all of the residues specified above and further below (i.e. all of the mutations in SEQ ID NO: 1 or 18 relative to SEQ ID NO: 6), in the positions specified herein, particularly the positions specified below, the variant may contain a conservative substitution of the amino acid residues at the equivalent positions in the RrgACatcher peptide (SEQ ID NO: 6). Thus, for example, if the residue at position 69 (or the equivalent position) is not isoleucine or phenylalanine, it is preferred that the residue represents a conservative substitution of the residue at the equivalent position in SEQ ID NO: 1, 6 or 18, e.g. leucine.

The term “linker” as used herein refers to molecules that function to link, i.e. conjugate or join, two molecules or components together, preferably by a covalent bond, e.g. an isopeptide bond. Thus, the polypeptide (peptide tag binding partner) of the invention and its peptide tag may be viewed as a two-part linker, wherein formation of the isopeptide bond between the first part, i.e. polypeptide, and second part, i.e. peptide tag, reconstitutes the linker, thereby joining molecules or components fused or conjugated to said first and second parts of the linker. Alternatively stated, the polypeptide (peptide tag binding partner) of the invention and its peptide tag may be viewed as a cognate pair that functions as a linker, i.e. a peptide tag and polypeptide cognate pair or a peptide tag and binding partner cognate pair. These terms are used interchangeably throughout the description.

The term “cognate” refers to components that function or specifically interact together. Thus, in the context of the present invention, a cognate pair may refer to a peptide tag and a polypeptide (peptide tag binding partner) of the invention that react together spontaneously to form an isopeptide bond. Thus, a two-part linker comprising a peptide tag and polypeptide that react together efficiently to form an isopeptide bond under conditions that enable the spontaneous formation of said isopeptide bond can also be referred to as being a “complementary pair”, i.e. a peptide tag and polypeptide complementary pair.

In some embodiments, a cognate pair refers to a peptide tag (i.e. DogTag or a variant thereof) and the polypeptide (affinity purification polypeptide) of the invention that bind non-covalently to form a complex (i.e. a polypeptide:peptide tag complex).

Thus, in some embodiments, a cognate peptide tag refers to a DogTag peptide or variant thereof, such as RrgATag or RrgATag2 (e.g. a peptide comprising an amino acid sequence set forth in any one of SEQ ID NOs: 3-5 or 17) with which the polypeptide of the invention reacts spontaneously to form an isopeptide bond. In some embodiments, the cognate peptide tag may be a peptide comprising an amino acid sequence with at least 80% (e.g. at least 85, 90 or 95%) sequence identity to an amino acid sequence as set forth in one of SEQ ID NOs: 3-5 or 17 that is capable of spontaneously forming an isopeptide bond with a polypeptide comprising an amino acid sequence as set forth in SEQ ID NO: 1, e.g. between an asparagine in the cognate peptide tag (i.e. an asparagine at a position equivalent to position 17 in any one of SEQ ID NOs: 3-5 or 17) and the lysine residue at position 9 of SEQ ID NO: 1.

In some embodiments, a cognate peptide tag refers to a peptide tag as defined herein (e.g. a peptide comprising an amino acid sequence set forth in one of SEQ ID NOs: 3-5 or 17) to which the polypeptide (affinity purification polypeptide) of the invention can bind selectively (e.g. specifically) and reversibly.

Thus, in some preferred embodiments, the peptide tag comprises or consists of an amino acid sequence as set forth in SEQ ID NO: 3, 4, 5 or 17 or an amino acid sequence with at least 80% sequence identity to a sequence as set forth in any one of SEQ ID NOs: 3-5 or 17 (e.g. at least 85, 90 or 95% identical to a sequence as set forth in any one of SEQ ID NOs: 3-5 or 17) wherein the amino acid sequence comprises an asparagine residue at position 17 and optionally comprises a threonine residue at position 5, an aspartic acid residue at position 10 and a glycine residue at position 11. Thus, in some embodiments the peptide tag comprises or consists of an amino acid sequence with at least 80% sequence identity to a sequence as set forth in SEQ ID NO: 3 (e.g. at least 85, 90 or 95% identical to a sequence as set forth in SEQ ID NO: 3) wherein the amino acid sequence comprises a threonine residue at position 5, an aspartic acid residue at position 10 a glycine residue at position 11 and an asparagine residue at position 17.

Thus, the invention further provides a two-part linker comprising a peptide (peptide tag) and polypeptide (a peptide tag binding partner), wherein:
a) said polypeptide (peptide tag binding partner) comprises an amino acid sequence as defined above (i.e. SEQ ID NO: 1 or a variant thereof); and
b) said peptide (peptide tag) comprises an amino acid sequence as defined above (e.g. an amino acid sequence as set forth in SEQ ID NO: 3, 4 or 5 or a variant thereof), 30
and wherein said peptide (peptide tag) and polypeptide (peptide tag binding partner) are capable of spontaneously forming an isopeptide bond between the asparagine residue in the peptide tag (e.g. at position 17 in SEQ ID NO: 3, 4 or 5) and the lysine residue at position 9 of SEQ ID NO: 1.

The lysine residue at position 9 of the polypeptide (peptide tag binding partner) of the invention (e.g. SEQ ID NO: 1) spontaneously forms an isopeptide bond with the asparagine residue at position 17 in SEQ ID NO: 3, 4, 5 or 17 under various conditions including those explained below that are suitable for the formation of an isopeptide bond between said peptide tag and polypeptide (peptide tag binding partner). It is evident from the Examples below that the polypeptide (peptide tag binding partner) of the invention is active under a range of conditions and capable of reacting with a variety of peptide tags (particularly SEQ ID NOs: 3-5).

For instance, the polypeptide (peptide tag binding partner) is active (i.e. capable of spontaneously forming an isopeptide bond with a peptide tag as described herein) in a variety of buffers including phosphate buffered saline (PBS), 4-(2-hydroxyethyl)-1-piperazineethanesulfonic acid (HEPES), HEPES buffered saline (HBS), Tris (tris(hydroxymethyl)aminomethane) and Tris buffered saline (TBS), both with and without EDTA. The polypeptide (peptide tag binding partner) is active at a pH of about 5.5-11.0, e.g. 5.5-10.0, 6.0-9.5, such as about 6.0-8.5 or 6.5-9.0, over a wide range of temperatures, e.g. 0-40° C., e.g. 1, 2, 3, 4, 5, 10, 12, 15, 18, 20, 22, 25, 28, 30, 35 or 37° C., preferably about 25-35° C., e.g. about 25° C. The polypeptide (peptide tag binding partner) of the invention is also active in the presence of the commonly used detergents, such as Tween 20 and Triton X-100, e.g. up to a concentration of about 1% (v/v), and in the presence of a reducing agent, e.g. dithiothreitol (DTT). The skilled person would readily be able to determine other suitable conditions.

Thus, in some embodiments, conditions that are suitable for the formation of an isopeptide bond between the polypeptide (peptide tag binding partner) of the invention and a cognate peptide tag (e.g. a peptide comprising or consisting of an amino acid sequence as set forth in SEQ ID NOs: 3-5) includes any conditions in which contacting the peptide tag and polypeptide (peptide tag binding partner) of the invention results in the spontaneous formation of an isopeptide bond between said peptide tag and polypeptide (peptide tag binding partner), particularly between the asparagine residue at position 17 of SEQ ID NO: 3, 4 or 5 and the lysine residue at position 9 of SEQ ID NO: 1 (or equivalent position). For instance, contacting said peptide tag and polypeptide (peptide tag binding partner) in buffered conditions, e.g. in a buffered solution or on a solid phase (e.g. column) that has been equilibrated with a buffer, such as PBS. The step of contacting may be at any suitable pH, such as about pH 5.5-11.0, e.g. 5.5-10.0, such as about pH 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4 or 9.6. Additionally or alternatively, the step of contacting may be at any suitable temperature, such as about 0-40° C., e.g. about 1-39, 2-38, 3-37, 4-36, 5-35, 6-34, 7-33, 8-32, 9-31 or 10-30° C., e.g. about 10, 12, 15, 18, 20, 22, 25, 28, 30, 33, 35 or 37° C., preferably about 25-35° C., e.g. about 25° C.

As noted above, the formation of the isopeptide bond between the peptide tag described herein and polypeptide (peptide tag binding partner) of the invention is spontaneous. In this respect, the polypeptide (peptide tag binding partner) comprises a glutamic acid at position 70 (or an equivalent position, based on the numbering of SEQ ID NO: 1) that facilitates, e.g. induces, promotes or catalyses, the formation of the isopeptide bond between the asparagine and lysine residues in the peptide tag and polypeptide (peptide tag binding partner), respectively.

The term “spontaneous” as used herein refers to an isopeptide bond, which can form in a protein or between peptides or proteins (e.g. between two peptides or a peptide and a protein, i.e. the peptide tag and polypeptide (peptide tag binding partner) of the invention) without any other agent (e.g. an enzyme catalyst) being present and/or without chemical modification of the protein or peptide, e.g. without native chemical ligation or chemical coupling using 1-ethyl-3-(3-dimethylaminopropyl) carbodiimide (EDC). Thus, native chemical ligation to modify a peptide or protein having a C-terminal thioester is not carried out.

Thus, a spontaneous isopeptide bond can form between a peptide tag as defined herein and a polypeptide (peptide tag binding partner) of the invention when in isolation and without chemical modification of the peptide tag and/or polypeptide of the invention. A spontaneous isopeptide bond may therefore form of its own accord in the absence of enzymes or other exogenous substances and without chemical modification of the peptide tag and/or polypeptide of the invention.

A spontaneous isopeptide bond may form almost immediately after contact of the peptide tag and polypeptide (peptide tag binding partner) of the invention, e.g. within 1, 2, 3, 4, 5, 10, 15, 20, 25 or 30 minutes, or within 1, 2, 4, 8, 12, 16, 20 or 24 hours.

The speed of isopeptide formation will be dependent on the concentration of the peptide tag and polypeptide reactants and the conditions of the reaction, e.g. temperature. In some embodiments, spontaneous isopeptide bond formation may complete for about 80% or more of the reactants in about 20 minute or less, e.g. where the reactants are each present at a concentration of about 5 μM at a reaction temperature of about 25° C.

Alternatively viewed, in some embodiments, spontaneous isopeptide bond formation may complete for about 80% or more of the reactants in about 20 minutes or less, e.g. where the reactants are each present at a concentration of about UM at a reaction temperature of about 25° C.

The other reaction conditions, e.g. buffer, pH etc. used to determine the speed of reaction defined above may be any conditions defined herein. In some embodiments, the reaction conditions are those used in the Examples. For instance, in some embodiments, the spontaneous isopeptide bond formation is complete in the amounts specified above in PBS buffer at a pH of about 7.5.

The polypeptide of the invention encompasses mutant forms of the polypeptide (i.e. peptide tag binding partner or affinity purification polypeptide) (i.e. referred to herein as homologues, variants or derivatives), which are structurally similar to the exemplified polypeptides set forth in SEQ ID NOs: 1 and 18. The polypeptide (peptide tag binding partner) variants of the invention are able to function as a peptide tag binding partner (catcher), i.e. capable of spontaneously forming an isopeptide bond between the asparagine at position 17 (or equivalent position) of a peptide tag as defined herein and the lysine at position 9 (or equivalent position) of the polypeptide (peptide tag binding partner) variant under suitable conditions as defined above. The affinity purification polypeptide variants of the invention are able to bind selectively and reversibly to the cognate peptide tag under suitable conditions as defined herein.

In cases where a polypeptide variant comprises mutations, e.g. deletions or insertions, relative to SEQ ID NO: 1 or 18, the residues specified above are present at equivalent amino acid positions in the variant polypeptide sequence. In some embodiments, deletions in the polypeptide variants of the invention are not N-terminal and/or C-terminal truncations.

However, as mentioned above, it is contemplated that the polypeptide exemplified herein (e.g. SEQ ID NO: 1 or 18) may be truncated at the N-terminus and/or C-terminus without significantly reducing the activity or function of the polypeptide. In particular, SEQ ID NO: 1 or 18 may be truncated by up to 4 amino acids at the N-terminus (e.g. 1, 2, 3 or 4 amino acids) and/or by up to 5 amino acids at the C-terminus (e.g. 1, 2, 3, 4 or 5 amino acids). Thus, the term variant as used herein includes truncation variants of the exemplified polypeptides. Alternatively viewed, the invention may be seen to provide a portion of the exemplified polypeptide, wherein said portion comprises an amino acid sequence as set forth in SEQ ID NO: 2 or 19 or a variant thereof, as discussed above.

As referred to herein a “portion” comprises at least an amino acid sequence as set forth in, e.g. SEQ ID NO: 2 or 19, i.e. at least 95, 96, 97, 98, 99, 100, 101, 102, or 103 amino acids of SEQ ID NO: 1 or 18 (the sequence from which it is derived) containing an amino acid sequence as set forth in SEQ ID NO: 2. Thus, said portion may be obtained from a central or N-terminal or C-terminal portion of the sequence. Preferably said portion is obtained from the central portion, i.e. it comprises an N-terminal and/or C-terminal truncation, as defined above. Notably, “portions” as described herein are polypeptides of the invention and therefore satisfy the identity (relative to a comparable region) conditions and functional equivalence conditions mentioned herein.

In some embodiments, a peptide tag for use with the polypeptides of the invention may be a variant of the sequences described herein, e.g. may differ from SEQ ID NOs: 3-5 by for example 1 to 5, 1 to 4, e.g. 1, 2 to 3 amino acid substitutions, insertions and/or deletions, preferably substitutions, as defined above. In some embodiments, the polypeptide variant of the present invention may differ from, e.g. SEQ ID NO: 1 or 18 as defined above. However, the peptide and polypeptide variants must retain their functional activity, e.g. their ability to spontaneously form an isopeptide bond with their cognate binding partner and peptide, respectively, or their ability to form a complex (i.e. a polypeptide:peptide tag complex).

Sequence identity may be determined by any suitable means known in the art, e.g. using the SWISS-PROT protein sequence databank using FASTA pep-cmp with a variable pamfactor, and gap creation penalty set at 12.0 and gap extension penalty set at 4.0, and a window of 2 amino acids. Other programs for determining amino acid sequence identity include the BestFit program of the Genetics Computer Group (GCG) Version 10 Software package from the University of Wisconsin. The program uses the local homology algorithm of Smith and Waterman with the default values: Gap creation penalty—8, Gap extension penalty=2, Average match=2.912, Average mismatch=−2.003.

Preferably said comparison is made over the full length of the sequence, but may be made over a smaller window of comparison, e.g. less than 100, 80 or 50 contiguous amino acids.

Preferably the peptide tag and polypeptide (peptide tag binding partner) variants (e.g. sequence identity-related variants) are functionally equivalent to the peptide tag and polypeptide (peptide tag binding partner) having a sequence as set forth in SEQ ID NOs: 3-5 or SEQ ID NOs: 1 or 2, respectively. As referred to herein, “functional equivalence” refers to variants of the peptide tag defined herein and polypeptide (peptide tag binding partner) of the invention discussed above that may show some reduced efficacy in the spontaneous formation of an isopeptide bond with its respective partner (e.g. lower expression yield, lower reaction rate, or activity in a limited range of reaction conditions (e.g. narrower temperature range, such as 10-30° C. etc.)) relative to the parent molecule (i.e. the molecule with which it shows sequence homology), but preferably are as efficient or are more efficient.

A mutant or variant peptide tag with activity that is “equivalent” to the activity of a peptide tag comprising or consisting of an amino acid sequence as set forth in one of SEQ ID NOs: 3-5 may have activity that is similar (i.e. comparable) to the activity of a peptide tag comprising or consisting of an amino acid sequence as set forth in one of SEQ ID NOs: 3-5, i.e. such that the practical applications of the peptide tag are not significantly affected, e.g. within a margin of experimental error. Thus, an equivalent peptide tag activity means that the mutant or variant peptide tag is capable of spontaneously forming an isopeptide bond with a polypeptide (peptide tag binding partner, e.g. comprising or consisting of an amino acid sequence as set forth in SEQ ID NO: 1 or 2) with a similar reaction rate (i.e. rate constant as discussed below) and/or yield to a peptide tag comprising or consisting of an amino acid sequence as set forth in one of SEQ ID NOs: 3-5 under the same conditions.

Similarly, a mutant or variant polypeptide of the invention with activity that is “equivalent” to the activity of a polypeptide comprising or consisting of an amino acid sequence as set forth in SEQ ID NO: 1, 2, 18 or 19 (preferably SEQ ID NO: 1 or 18) may have functional properties (e.g. solubility and/or activity (e.g. reactivity or affinity)) that are similar (i.e. comparable) to the properties of a polypeptide comprising or consisting of an amino acid sequence as set forth in SEQ ID NO: 1, 2, 18 or 19 (preferably SEQ ID NO: 1 or 18), i.e. such that the practical applications of the polypeptide are not significantly affected, e.g. within a margin of experimental error.

Thus, in some embodiments, an equivalent polypeptide (peptide tag binding partner) activity or function means that the mutant or variant polypeptide (peptide tag binding partner) of the invention is capable of spontaneously forming an isopeptide bond with a peptide tag (e.g. comprising or consisting of an amino acid sequence as set forth in one of SEQ ID NOs: 3-5) with a similar reaction rate (i.e. rate constant as discussed below) and/or yield to a polypeptide (peptide tag binding partner) comprising or consisting of an amino acid sequence as set forth in SEQ ID NO: 1 or 2 (preferably SEQ ID NO: 1) under the same conditions.

In some embodiments, an equivalent polypeptide function means that the mutant or variant polypeptide (e.g. peptide tag binding partner) of the invention has similar solubility characteristics to a polypeptide (peptide tag binding partner) comprising or consisting of an amino acid sequence as set forth in SEQ ID NO: 1, 2, 18 or 19 (preferably SEQ ID NO: 1 or 18) under the same conditions. Notably, in some embodiments, an equivalent polypeptide with similar solubility characteristics to a polypeptide (peptide tag binding partner) comprising or consisting of an amino acid sequence as set forth in SEQ ID NO: 1 or 2 (preferably SEQ ID NO: 1) must also be capable of spontaneously forming an isopeptide bond with a peptide tag as defined herein (e.g. comprising or consisting of an amino acid sequence as set forth in one of SEQ ID NOs: 3-5). Preferably, an equivalent polypeptide has similar solubility, reaction rate and/or yield to a polypeptide (peptide tag binding partner) comprising or consisting of an amino acid sequence as set forth in SEQ ID NO: 1 or 2 (preferably SEQ ID NO: 1) under the same conditions.

In some embodiments, an equivalent polypeptide with similar solubility characteristics to a polypeptide (affinity purification polypeptide) comprising or consisting of an amino acid sequence as set forth in SEQ ID NO: 18 or 19 (preferably SEQ ID NO: 18) must also be capable of binding selectively and reversibly to the cognate peptide tag under suitable conditions as defined herein. Preferably, an equivalent polypeptide has similar solubility, binding affinity and/or yield to a polypeptide (affinity purification polypeptide) comprising or consisting of an amino acid sequence as set forth in SEQ ID NO: 18 or 19 (preferably SEQ ID NO: 18) under the same conditions.

Accordingly, it can be seen that a mutant or variant polypeptide of the invention may have solubility that is similar (i.e. comparable) to the solubility of a polypeptide comprising or consisting of an amino acid sequence as set forth in SEQ ID NO: 1, 2, 18 or 19 (preferably SEQ ID NO: 1 or 18), i.e. such that the practical applications of the polypeptide are not significantly affected, e.g. within a margin of experimental error.

The activity of different peptide tag and polypeptides (e.g. SEQ ID NO: 3 versus mutant and SEQ ID NO: 1 versus mutant, respectively) measured under the same reaction conditions, e.g. temperature, substrates (i.e. peptide tag or polypeptide sequences) and their concentration, buffer, salt etc. as exemplified above, can be readily compared to determine whether the activity for each peptide tag and polypeptide is higher, lower or equivalent.

In particular, the peptide tag variants defined herein and the polypeptide variants of the invention may have an equivalent rate constant to the peptide tag and polypeptide having a sequence as set forth in SEQ ID NOs: 3-5 or SEQ ID NOs: 1 or 2, respectively. The rate constant refers to the coefficient of proportionality relating the rate of the reaction (the formation of an isopeptide bond) at a given temperature to the product of the concentrations of reactants (i.e. the product of the concentration of the peptide tag and polypeptide of the invention).

Thus, the activity, e.g. rate constant, of the variant (e.g. mutant) peptide tag disclosed herein may be at least 60%, e.g. at least 70, 75, 80, 85 or 90% of the activity, e.g. rate constant, of a peptide tag comprising or consisting of an amino acid sequence as set forth in one of SEQ ID NOs: 3-5, such as at least 91, 92, 93, 94 or 95% of the activity of a peptide tag comprising or consisting of an amino acid sequence as set forth in one of SEQ ID NOs: 3-5. Alternatively viewed, the activity, e.g. rate constant, of the mutant peptide tag may be no more than 40% lower than the activity, e.g. rate constant, of a peptide tag comprising or consisting of an amino acid sequence as set forth in one of SEQ ID NOs: 3-5, e.g. no more than 35, 30, 25 or 20% lower than the activity, e.g. rate constant, of a peptide tag comprising or consisting of an amino acid sequence as set forth in one of SEQ ID NOs: 3-5, such as no more than 10, 9, 8, 7, 6 or 5% lower than the activity, e.g. rate constant, of a peptide tag comprising or consisting of an amino acid sequence as set forth in one of SEQ ID NOs: 3-5.

Similarly, the activity, e.g. rate constant, of the variant polypeptide (peptide tag binding partner) of the invention may be at least 60%, e.g. at least 70, 75, 80, 85 or 90% of the activity, e.g. rate constant, of a polypeptide comprising or consisting of an amino acid sequence as set forth in SEQ ID NO: 1 or 2, such as at least 91, 92, 93, 94, 95, 96, 97, 98 or 99% of the activity, e.g. rate constant, of a polypeptide comprising or consisting of an amino acid sequence as set forth in SEQ ID NO: 1 or 2. Alternatively viewed, the activity of the variant polypeptide may be no more than 40% lower than the activity, e.g. rate constant, of a polypeptide comprising or consisting of an amino acid sequence as set forth in SEQ ID NO: 1 or 2, e.g. no more than 35, 30, 25 or 20% lower than the activity, e.g. rate constant, of a polypeptide comprising or consisting of an amino acid sequence as set forth in SEQ ID NO: 1 or 2, such as no more than 10, 9, 8, 7, 6, 5, 4, 3, 2 or 1% lower than the activity, e.g. rate constant, of a polypeptide comprising or consisting of an amino acid sequence as set forth in SEQ ID NO: 1 or 2.

Moreover, the solubility of the variant polypeptide of the invention may be at least 60%, e.g. at least 70, 75, 80, 85 or 90% of the solubility of a polypeptide comprising or consisting of an amino acid sequence as set forth in SEQ ID NO: 1, 2, 18 or 19, such as at least 91, 92, 93, 94, 95, 96, 97, 98 or 99% of the solubility of a polypeptide comprising or consisting of an amino acid sequence as set forth in SEQ ID NO: 1 or 2, when measured under the same conditions, e.g. buffer, temperature, pH etc. Alternatively viewed, the solubility of the variant polypeptide may be no more than 40% lower than the solubility of a polypeptide comprising or consisting of an amino acid sequence as set forth in SEQ ID NO: 1, 2, 18 or 19, e.g. no more than 35, 30, 25 or 20% lower than the solubility of a polypeptide comprising or consisting of an amino acid sequence as set forth in SEQ ID NO: 1, 2, 18 or 19, such as no more than 10, 9, 8, 7, 6, 5, 4, 3, 2 or 1% lower than the solubility of a polypeptide comprising or consisting of an amino acid sequence as set forth in SEQ ID NO: 1, 2, 18 or 19, when measured under the same conditions, e.g. buffer, temperature, pH etc. Solubility of a polypeptide may be measured using any suitable means known in the art. For instance, as shown in the Examples, solubility may be measured by determining the yield of soluble protein obtained from expression in a suitable host cell, e.g. E. coli, under a specified conditions. In a further representative example, relative solubility of proteins may be measured using a spin concentrator, wherein protein solutions are concentrated until protein aggregation occurs and the point of aggregation may be used to determine the relative solubility of the proteins.

Notably, the rate constant of the reaction of the peptide tag disclosed herein and the polypeptide of the invention may be lower than the values described in the Examples when the peptide tag and/or polypeptide are fused to large molecules or components (e.g. proteins), which diffuse slower than the isolated peptide tag and polypeptide. Moreover, the rate constant may be reduced if the molecules or components to which the peptide tag and/or polypeptide are fused cause steric hindrance to the reaction. Accordingly, when measuring the rate constant of the reaction of the peptide tag variants disclosed herein and the polypeptide variants of the invention, it is preferred that measurement is performed using isolated peptide tags and polypeptides, i.e. peptide tags and polypeptides that are not fused or conjugated to other molecules or components.

However, as shown in the Examples, it is often convenient to measure the rate constant of the reaction of the polypeptide variants of the invention using a peptide tag that is fused to a polypeptide. Thus, when measuring and comparing the rate constants of different polypeptide variants using a peptide tag that is fused to a polypeptide, it is preferred that a polypeptide fused to the peptide tag is the same size, preferably the same sequence, in all reactions.

It will be evident that fusion to large molecules or components and/or steric hindrance will also affect the rate constant of other peptide tags and polypeptides, e.g. RrgATag, RrgATag2 and RrgACatcher. Thus, the enhancements in rate constant of the polypeptide of the invention may still be advantageous when the polypeptide of the invention and its cognate peptide tag are used at high concentrations, such as about at least 10 μM, (e.g. when fused to large molecules or components) in addition to their use at low concentrations.

The reaction rate and rate constant can be assessed by any suitable means known in the art and as described in the Examples and in WO 2018/197854 (herein incorporated by reference). For instance, the reaction rate may be monitored by (i) assessing the mobility of the reaction products on SDS-PAGE after boiling in SDS or other strong denaturing treatment that would disrupt all non-covalent interactions or (ii) by mass spectrometry.

Hence, any modification or combination of modifications may be made to SEQ ID NO: 1 to produce a variant polypeptide (peptide tag binding partner) of the invention, provided that the variant polypeptide (peptide tag binding partner) comprises a lysine residue at a position equivalent to position 9 of SEQ ID NO: 1 and a glutamic acid residue at a position equivalent to position 70 of SEQ ID NO: 1 and at least one (preferably two or more) other amino acid residue(s) at positions equivalent to positions 4, 11, 13, 47, 59, 69, 75, 87, 89 and 92 of SEQ ID NO: 1 as defined above (including that wherein the at least one amino acid is proline at position 75, the amino acid sequence also comprises at least one other amino acid residue at positions equivalent to positions 4, 11, 13, 47, 59, 69, 87, 89 and 92 of SEQ ID NO: 1 as defined above) and retains the functional characteristics defined above, i.e. it results in a polypeptide (peptide tag binding partner) capable of spontaneously forming an isopeptide bond with a peptide tag comprising or consisting of an amino acid sequence as set forth in one of SEQ ID NOs: 3-5 and optionally has an equivalent or higher yield, reaction rate, e.g. rate constant, solubility, tolerance to variation in temperature and/or buffer relative to a polypeptide (peptide tag binding partner) having an amino acid sequence as set forth in SEQ ID NO: 1.

In some further embodiments, the variant polypeptide of the invention comprises the residues specified above and retains the functional characteristics defined above, i.e. it results in a polypeptide (peptide tag binding partner) capable of spontaneously forming an isopeptide bond with a peptide tag comprising or consisting of an amino acid sequence as set forth in one of SEQ ID NOs: 3-5 and optionally has an equivalent or higher yield, reaction rate, e.g. rate constant, solubility, tolerance to variation in temperature and/or buffer range relative to a polypeptide (peptide tag binding partner) having an amino acid sequence as set forth in SEQ ID NO: 1.

Alternatively viewed, any modification or combination of modifications (preferably substitutions) may be made to SEQ ID NO: 2 to produce a variant polypeptide (peptide tag binding partner) of the invention, provided that the variant polypeptide (peptide tag binding partner) comprises a lysine residue at a position equivalent to position 5 of SEQ ID NO: 2 and a glutamic acid residue at a position equivalent to position 66 of SEQ ID NO: 2 and at least one (preferably two or more) other amino acid residue(s) at positions equivalent to positions 7, 9, 43, 55, 65, 71, 83, 85 and 88 of SEQ ID NO: 2 as defined above (including that wherein the at least one amino acid is proline at position 71, the amino acid sequence also comprises at least one other amino acid residue at positions equivalent to positions 7, 9, 43, 55, 65, 71, 83, 85 and 88 of SEQ ID NO: 2 as defined above) and retains the functional characteristics defined above, i.e. it results in a polypeptide (peptide tag binding partner) capable of spontaneously forming an isopeptide bond with a peptide tag comprising or consisting of an amino acid sequence as set forth in one of SEQ ID NOs: 3-5 and optionally has an equivalent or higher yield, reaction rate, e.g. rate constant, solubility, tolerance to variation in temperature and/or buffer range relative to a polypeptide (peptide tag binding partner) having an amino acid sequence as set forth in SEQ ID NO: 2.

In some further embodiments, the truncated variant polypeptide of the invention comprises the residues specified above and retains the functional characteristics defined above, i.e. it results in a polypeptide (peptide tag binding partner) capable of spontaneously forming an isopeptide bond with a peptide tag comprising or consisting of an amino acid sequence as set forth in one of SEQ ID NOs: 3-5 and optionally has an equivalent or higher yield, reaction rate, e.g. rate constant, solubility, tolerance to variation in temperature and/or buffer range relative to a polypeptide (peptide tag binding partner) having an amino acid sequence as set forth in SEQ ID NO: 2.

An equivalent position in the peptide tag disclosed herein is preferably determined by reference to the amino acid sequence of SEQ ID NO: 3. An equivalent position in the polypeptide (peptide tag binding partner) of the invention is determined by reference to the amino acid sequence of SEQ ID NO: 1 or 2. The homologous or corresponding position can be readily deduced by lining up the sequence of the homologue (mutant, variant or derivative) peptide tag and the sequence of SEQ ID NO: 3 or the sequence of the homologue (mutant, variant or derivative) polypeptide (peptide tag binding partner) and the sequence of SEQ ID NO: 1 or 2 based on the homology or identity between the sequences, for example using a BLAST algorithm.

The terms “tag” and “peptide tag” as used herein generally refer to a peptide or oligopeptide.

The term “peptide tag binding partner”, “binding partner” or “catcher” as used herein generally refers to a polypeptide or protein.

In this respect, there is no standard definition regarding the size boundaries between what is meant by peptide and what is meant by polypeptide. Typically a peptide may be viewed as comprising between 2-39 amino acids. Accordingly, a polypeptide may be viewed as comprising at least 40 amino acids, preferably at least 50, 60, 70, 80, 90 or 100 amino acids.

Thus, in preferred embodiments a peptide tag as defined herein may be viewed as comprising at least 12 amino acids, e.g. 12-39 amino acids, such as e.g. 13-35, 14-34, 15-33, 16-31, 17-30 amino acids in length, e.g. it may comprise or consist of 12, 13, 14, 15, 16, 17, 18, 19, 20, 21, 22 or 23 amino acids.

A polypeptide of the invention (e.g. a peptide tag binding partner, binding partner or “catcher” or affinity purification polypeptide) as defined herein may be viewed as comprising at least 95 amino acids, e.g. 95-150 amino acids, such as e.g. 95-140, 95-130, 95-120 amino acids in length, e.g. it may comprise or consist of 95, 96, 97, 98, 99, 100, 101, 102, 103 or 104 amino acids.

As discussed above, two-part linkers (e.g. tag and catcher systems or pairs, i.e. cognate pairs) have a large number of utilities and the polypeptide (peptide tag binding partner) of the invention and its cognate peptide tag(s) (e.g. SEQ ID NOs: 3-5) find particular utility in conjugating (i.e. joining or linking) two molecules or components via an isopeptide bond. For instance, the peptide tag and polypeptide (peptide tag binding partner) may be separately conjugated or fused to molecules or components of interest and subsequently contacted together under conditions suitable to allow the spontaneous formation of an isopeptide bond between the peptide tag and polypeptide (peptide tag binding partner), thereby joining (i.e. linking or conjugating) the molecules or components via an isopeptide bond.

Thus, in some embodiments, the invention may be seen to provide the use of a peptide (peptide tag) and polypeptide (peptide tag binding partner) pair as defined herein to conjugate two molecules or components via an isopeptide bond,
wherein said molecules or components conjugated via an isopeptide bond comprise:
a) a first molecule or component comprising (e.g. conjugated or fused to) a polypeptide (peptide tag binding partner) of the invention as defined herein; and
b) a second molecule or component comprising (e.g. conjugated or fused to) a peptide (peptide tag) as defined herein.

It will be evident that the use of the peptide tag and polypeptide (peptide tag binding partner) pair (i.e. two-part linker) described above comprises contacting said first and second molecules under conditions suitable to enable (e.g. promote or facilitate) the spontaneous formation of an isopeptide bond between said peptide tag and polypeptide (peptide tag binding partner) as described above.

As noted above, the peptide tag and polypeptide (peptide tag binding partner) pair (i.e. two-part linker) described above are particularly effective when the peptide tag is incorporated into a molecule or component at an internal site, i.e. not at one of the termini of said molecule or component. Accordingly, in some embodiments, the second molecule or component may comprise a peptide (peptide tag) as defined herein at an internal site, e.g. in a loop. Alternatively put, the peptide tag as defined herein may be present at an internal site in the second molecule or component.

The term “internal site” as used herein refers to a site within a molecule or component into which the peptide tag or polypeptide (peptide tag binding partner) of the present invention is to be incorporated which is not at either of the termini of said molecule or component, i.e. which is internal within said molecule or component. Where the molecule or component is a protein, an internal site may be a site that is at least 1 or more residues away from a terminus of the protein, e.g. at least 2, 3, 4, 5, 10, 15, 20 or 25 or more residues away from a terminus of the protein. The internal site may be at any point internally within the molecule or component. Where the molecule or component is a protein, it is preferred if the internal site is within a protein loop region, i.e. within a region which connects two regions of defined regular secondary structure within the protein.

Accordingly, in some embodiments, the second molecule or component may be a protein, and may comprise a peptide (peptide tag) as defined herein at an internal site. In preferred embodiments, the second molecule or component may be a protein, and may comprise a peptide (peptide tag) as defined herein in a loop region or domain.

Alternatively viewed, the invention provides a process for conjugating two molecules or components via an isopeptide bond comprising:
a) providing a first molecule or component comprising (e.g. conjugated or fused to) a polypeptide (peptide tag binding partner) of the invention as defined herein;
b) providing a second molecule or component comprising (e.g. conjugated or fused to) a peptide (peptide tag) as defined herein;
c) contacting said first and second molecules or components under conditions that enable (e.g. promote or facilitate) the spontaneous formation of an isopeptide bond between the peptide and polypeptide as described above, thereby conjugating said first molecule or component to said second molecule or component via an isopeptide bond to form a complex.

Again, in some embodiments, the second molecule or component may comprise a peptide (peptide tag) as defined herein at an internal site. In a preferred embodiment the second molecule or component is a protein, and the peptide tag as defined herein is present at an internal site of said protein, preferably in a loop region or domain.

The terms “conjugating” or “linking” in the context of the present invention with respect to connecting two or more molecules or components to form a complex refers to joining or conjugating said molecules or components, e.g. proteins, via a covalent bond, particularly an isopeptide bond which forms between the peptide tag and polypeptide (peptide tag binding partner) that are incorporated in, or fused to, said molecules or components, e.g. proteins (e.g. the peptide tag and polypeptide (peptide tag binding partner)) may form domains of proteins to be conjugated or linked together).

As mentioned above, in some embodiments, the peptide tag disclosed herein and/or polypeptide of the invention are fused or conjugated to other molecules or to other components or entities. Such molecules or components (i.e. entities) may be a nucleic acid molecule, protein (e.g. antibody or antigen-binding fragment thereof), peptide, small-molecule organic compound, fluorophore, metal-ligand complex, polysaccharide, nanoparticle, 2D monolayer (e.g. graphene), lipid, nanotube, polymer, cell, virus, virus-like particle, viral vector or any combination of these. In some embodiments the component or entity to which the peptide tag and/or polypeptide is fused or conjugated is a solid support, i.e. solid substrate or solid phase, as defined below.

Thus, alternatively viewed, the invention provides a nucleic acid molecule, protein (e.g. antibody or antigen-binding fragment thereof), peptide, small-molecule organic compound, fluorophore, metal-ligand complex, polysaccharide, nanoparticle, 2D monolayer (e.g. graphene), lipid, nanotube, polymer, cell, virus, virus-like particle, viral vector or any combination thereof or solid support fused or conjugated to a peptide tag and/or polypeptide of the invention.

The cell may be a prokaryotic or eukaryotic cell. In some embodiments, the cell is a prokaryotic cell, e.g. a bacterial cell. In some embodiments, the cell is a eukaryotic cell, such as an animal cell, e.g. a human cell.

In some embodiments, the peptide tag and/or polypeptide (e.g. peptide tag binding partner) may be conjugated or fused to a compound or molecule which has a therapeutic or prophylactic effect, e.g. an antibiotic, antiviral, vaccine, antitumour agent, e.g. a radioactive compound or isotope, cytokines, toxins, oligonucleotides and nucleic acids encoding genes or nucleic acid vaccines.

In some embodiments, the peptide tag and/or polypeptide (e.g. peptide tag binding partner) may be conjugated or fused to a label, e.g. a radiolabel, a fluorescent label, luminescent label, a chromophore label as well as to substances and enzymes which generate a detectable signal, e.g. horseradish peroxidase, luciferase or alkaline phosphatase. This detection may be applied in numerous assays where antibodies are conventionally used, including Western blotting/immunoblotting, histochemistry, enzyme-linked immunosorbent assay (ELISA), or flow cytometry (FACS) formats. Labels for magnetic resonance imaging, positron emission tomography probes and boron 10 for neutron capture therapy may also be conjugated to the peptide tag and/or polypeptide (peptide tag binding partner) of the invention. Particularly, the peptide tag and/or polypeptide (e.g. peptide tag binding partner) may be fused or produced with another peptide, for example His tag, and/or may be fused or produced with another protein, for example with the purpose of enhancing recombinant protein expression by fusing to Maltose Binding Protein.

In some embodiments, the polypeptide of the invention may comprise a peptide (e.g. a c-myc Tag) fused to its C-terminus, e.g. via linker or spacer sequence such as SEQ ID NO: 16, which may further improve the solubility of the polypeptide, e.g. relative to the polypeptide without the C-terminal peptide.

In some embodiments, it may be useful to introduce a cysteine residue into the polypeptide of the invention to couple the polypeptide to another molecule or component, such as a label, e.g. a fluorescent label, or a solid substrate. For instance, the introduction of a cysteine residue would allow the polypeptide to be coupled to another molecule or component, such as a label, e.g. a fluorescent label, containing a maleimide functional group.

As noted above, the peptide tag binding partner polypeptide (“catcher”) defined above may be modified to abrogate spontaneous formation of an isopeptide bond between the polypeptide and its cognate peptide tag. Advantageously, the modified polypeptide may be immobilised on a solid substrate (phase) to provide an affinity purification system for the isolation and/or purification of molecules or components comprising a peptide tag as defined herein. Thus, any of the polypeptides defined above may be modified to provide a polypeptide with utility in an affinity purification system by substituting the glutamic acid at position 70 of SEQ ID NO: 1 or position 66 of SEQ ID NO: 2 (or an equivalent position), such that the modified polypeptide cannot spontaneously form an isopeptide bond with a peptide tag as defined herein. Thus, the modified polypeptide (i.e. the affinity purification polypeptide) may comprise a non-conservative substitution of the glutamic acid at position 70 of SEQ ID NO: 1 or position 66 of SEQ ID NO: 2 (or an equivalent position). Alternatively viewed, the modified polypeptide does not contain a glutamic acid or aspartic acid at position 70 of SEQ ID NO: 1 or position 66 of SEQ ID NO: 2 (or an equivalent position). In some embodiments, glutamic acid at position 70 of SEQ ID NO: 1 or position 66 of SEQ ID NO: 2 (or an equivalent position) may be substituted with alanine, glycine, serine, asparagine, or threonine.

Thus, in one embodiment, the invention provides a polypeptide (affinity purification polypeptide) comprising:
i) an amino acid sequence as set forth in SEQ ID NO: 18, wherein X at position 70 is not glutamic acid or aspartic acid (i.e. any amino acid other than glutamic acid or aspartic acid), optionally wherein X at position 70 is selected from alanine, glycine, serine, asparagine, or threonine;
ii) a portion of (i) comprising an amino acid sequence as set forth in SEQ ID NO: 19, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 18, wherein X at position 70 is not glutamic acid or aspartic acid, optionally wherein X at position 70 is selected from alanine, glycine, serine, asparagine, or threonine and wherein the amino acid sequence comprises one or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein if the amino acid sequence comprises proline at position 75, it also comprises one or more amino acid residues selected from 1)-6) and 8)-10), and wherein the specified amino acid residues are at positions equivalent to the positions in SEQ ID NO: 18; or
iv) a portion of (iii) comprising an amino acid sequence with at least 80% sequence identity to a sequence as set forth in SEQ ID NO: 19, wherein X at position 66 is not glutamic acid or aspartic acid, optionally wherein X at position 66 is selected from alanine, glycine, serine, asparagine, or threonine and wherein the amino acid sequence comprises one or more of the following:
1) aspartic acid at position 7;
2) threonine at position 9;
3) aspartic acid at position 43;
4) threonine at position 55;
5) isoleucine at position 65;
6) proline at position 71;
7 serine at position 83;
8) arginine at position 85; and
9) aspartic acid at position 88;
wherein if the amino acid sequence comprises proline at position 71, it also comprises one or more amino acid residues selected from 1)-5) and 7)-9), and wherein the specified amino acid residues are at positions equivalent to the positions in SEQ ID NO: 19,
and wherein the polypeptide binds selectively and reversibly to a peptide comprising an amino acid sequence as set forth in SEQ ID NO: 3.

In a further embodiment, the invention provides a polypeptide (affinity purification polypeptide) comprising:
i) an amino acid sequence as set forth in SEQ ID NO: 18, wherein X at position 70 is not glutamic acid or aspartic acid (i.e. any amino acid other than glutamic acid or aspartic acid), optionally wherein X at position 70 is selected from alanine, glycine, serine, asparagine, or threonine;
ii) a portion of (i) comprising an amino acid sequence as set forth in SEQ ID NO: 19, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 18, wherein X at position 70 is not glutamic acid or aspartic acid, optionally wherein X at position 70 is selected from alanine, glycine, serine, asparagine, or threonine and wherein the amino acid sequence comprises one or more of the following:
1) glutamic acid at position 4;
2) aspartic acid at position 11;
3) threonine at position 13;
4) aspartic acid at position 47;
5) threonine at position 59;
6) proline at position 75; and
7) aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 19, wherein X at position 66 is not glutamic acid or aspartic acid, optionally wherein X at position 66 is selected from alanine, glycine, serine, asparagine, or threonine and wherein the amino acid sequence comprises one or more of the following:
1) aspartic acid at position 7;
2) threonine at position 9;
3) aspartic acid at position 43;
4) threonine at position 55;
5) proline at position 71; and
6) aspartic acid at position 88;
and one or more of the following:
1) isoleucine at position 65;
2) serine at position 83; and
3) arginine at position 85;
wherein the specified amino acid residues are at positions equivalent to the positions in SEQ ID NO: 19,
and wherein the polypeptide binds selectively and reversibly to a peptide comprising an amino acid sequence as set forth in SEQ ID NO: 3.

In some embodiments, X at position 70 of SEQ ID NO: 18 (or the equivalent position) is a conventional (standard) amino acid other than an acidic amino acid, i.e. X is not D or E.

In some embodiments, X at position 70 of SEQ ID NO: 18 (or the equivalent position) is not a basic amino acid (e.g. R, K or H), an aromatic amino acid (e.g. F, Y or W), cysteine (C) and/or proline (P).

Accordingly, in some embodiments, X at position 70 of SEQ ID NO: 18 (or the equivalent position) is selected from A, G, I, L, M, N, Q, S, T and V. In some embodiments, X at position 70 (or the equivalent position) is selected from A, G, S, N and T.

A “conventional or standard amino acid” is an amino acid that is used in vivo to produce a polypeptide or protein molecule, i.e. a proteinogenic amino acid. In other words, an amino acid with a standard or conventional R-group or an amino acid which possesses a side chain that is coded for by the standard genetic code, i.e. “coded amino acids”.

The combinations “solubility modifications” and/or “activity modifications” specified above with respect to the peptide tag binding partner polypeptide of the invention (i.e. the polypeptide capable of spontaneously forming an isopeptide bond with a peptide tag as defined herein, e.g. DogCatcher and functional variants thereof), apply equally to the modified (affinity purification) polypeptide defined above. For instance, the affinity purification polypeptide may comprise all of the “solubility modifications” and/or all of the “activity modifications”, or any selection or combination thereof as defined above.

The lysine residue at position 9 of SEQ ID NO: 1 may not be required in the affinity purification polypeptide of the invention because the polypeptide does not spontaneously form an isopeptide bond with a peptide tag as defined herein. However, as this residue may interact non-covalently with the peptide tag to facilitate selective binding of the polypeptide to the peptide tag, it may be advantageous to retain it in the affinity purification polypeptide of the invention.

Thus, in some embodiments, the (affinity purification) polypeptide defined above comprises an amino acid sequence with at least 80% sequence identity to a sequence as set forth in SEQ ID NO: 18 or 19 and wherein the amino acid sequence comprises lysine at a position equivalent to position 9 in SEQ ID NO: 18 or position 5 in SEQ ID NO: 19.

As noted above, it may be useful to introduce a cysteine residue into the polypeptide of the invention to couple the polypeptide to another molecule or component, particularly a solid substrate, e.g. for use in an affinity purification system or apparatus as defined herein. In some embodiments, the cysteine residue may be incorporated into the polypeptide by the addition of an N-terminal or C-terminal amino acid sequence (e.g. tag) comprising a cysteine residue, as shown in SEQ ID NO: 20.

Thus, in some embodiments, the polypeptide defined above (e.g. affinity purification polypeptide) comprises an additional N-terminal or C-terminal sequence comprising a cysteine residue.

In some embodiments, a cysteine residue may be introduced into the polypeptide by substituting an amino acid with a cysteine residue. In a preferred embodiment, the cysteine residue is not introduced at position of any of the solubility or activity modification residues defined above. In some embodiments, the aspartic acid at a position equivalent to position 31 in SEQ ID NO: 18 or a position equivalent to position 27 in SEQ ID NO: 19 is substituted with a cysteine residue. In some embodiments, the glutamine at a position equivalent to position 41 in SEQ ID NO: 18 or a position equivalent to position 37 in SEQ ID NO: 19 is substituted with a cysteine residue.

Thus, in some embodiments, the (affinity purification) polypeptide comprises a polypeptide comprising:
i) an amino acid sequence as set forth in SEQ ID NO: 21 or 22, wherein X at position 70 is not glutamic acid or aspartic acid, optionally wherein X at position 70 is selected from alanine, glycine, serine, asparagine, or threonine;
ii) a portion of (i) comprising an amino acid sequence as set forth in SEQ ID NO: 23 or 24, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 21 or 22, wherein X at position 70 is not glutamic acid or aspartic acid, optionally wherein X at position 70 is selected from alanine, glycine, serine, asparagine, or threonine and wherein the amino acid sequence comprises a cysteine at position 31 or 41 and one or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein the specified amino acid residues are at positions equivalent to the positions in SEQ ID NO: 21 or 22 and optionally wherein: (A) if the amino acid sequence comprises proline at position 75, it also comprises one or more amino acid residues selected from 1)-6) and 8)-10); or (B) the amino acid sequence comprises at least one amino acid residue selected from 1)-5), 7) and 10) and one amino acid residue selected from 6), 8) and 9); or
iv) a portion of (iii) comprising an amino acid sequence with at least 80% sequence identity to a sequence as set forth in SEQ ID NO: 23 or 24, wherein X at position 66 is not glutamic acid or aspartic acid, optionally wherein X at position 66 is selected from alanine, glycine, serine, asparagine, or threonine and wherein the amino acid sequence comprises a cysteine at position 27 or 37 and one or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein the specified amino acid residues are at positions equivalent to the positions in SEQ ID NO: 23 or 24 and optionally wherein: (A) if the amino acid sequence comprises proline at position 71, it also comprises one or more amino acid residues selected from 1)-5) and 7)-9); or (B) the amino acid sequence comprises at least one amino acid residue selected from 1)-4), 6) and 9) and 1 amino acid residue selected from 5), 7) and 8),
and wherein the polypeptide binds selectively and reversibly to a peptide comprising an amino acid sequence as set forth in SEQ ID NO: 3.

In some embodiments, the (affinity purification) polypeptide defined above comprises an amino acid sequence with at least 80% sequence identity to a sequence as set forth in any one of SEQ ID NOs: 21 to 24, wherein the amino acid sequence comprises lysine at a position equivalent to position 9 in SEQ ID NO: 21 or 22 or position 5 in SEQ ID NO: 23 or 24.

The term “binds selectively” refers to the ability of the (affinity purification) polypeptide to bind non-covalently (e.g. by van der Waals forces and/or hydrogen-bonding) to its cognate peptide tag with greater affinity and/or specificity than to other components in the sample in which the peptide tag is present (e.g. the sample from which the peptide tag (and associated molecule or component to which the peptide tag is fused or conjugated, i.e. fusion partner) is to be isolated or purified). Thus, the (affinity purification) polypeptide of the invention may alternatively be viewed as binding specifically and reversibly to its cognate peptide tag (i.e. DogTag peptide or a variant thereof), such as a peptide comprising an amino acid sequence as set forth in SEQ ID NOs: 3, 4, 5 or 17, under suitable conditions.

Binding to the cognate peptide tag may be distinguished from binding to other molecules (e.g. peptides or polypeptides) present in the sample, i.e. non-cognate molecules. The (affinity purification) polypeptide of the invention either does not bind to other molecules (e.g. peptides or polypeptides) present in the sample or does so negligibly or non-detectably that any such non-specific binding, if it occurs, readily may be distinguished from binding to the cognate peptide tag.

In particular, if the (affinity purification) polypeptide of the invention binds to molecules other than the cognate peptide tag, such binding must be transient and the binding affinity must be less than the binding affinity of the (affinity purification) polypeptide for the cognate peptide tag. Thus, the binding affinity of the (affinity purification) polypeptide for the peptide tag should be at least an order of magnitude more than the other molecules (i.e. non-cognate molecules) present in the sample. Preferably, the binding affinity of the (affinity purification) polypeptide for the cognate peptide tag should be at least 2, 3, 4, 5, or 6 orders of magnitude more than the binding affinity for non-cognate molecules (e.g. peptides or polypeptides).

Thus, selective or specific binding refers to affinity of the (affinity purification) polypeptide of the invention for its cognate peptide tag where the dissociation constant of the polypeptide for the cognate peptide tag is less than about 10⁻³ M. In a preferred embodiment the dissociation constant of the polypeptide for its cognate peptide tag is less than about 10⁻⁴ M, 10⁻⁵ M, 10⁻⁶ M, 10⁻⁷M, 10⁻⁸ M or 10⁻⁹ M.

The binding selectivity (e.g. specificity) of the (affinity purification) polypeptide of the invention may also be defined based on the yield and/or purity of the product, i.e. the cognate peptide tag and associated molecule or component (fusion partner, e.g. polypeptide), to which the peptide tag is fused or conjugated, obtained in the isolation or purification process defined below. In some embodiments, the (affinity purification) polypeptide of the invention in the process defined below results in a product with a purity of at least about 75%, such as at least about 80%, 85% or 90%. The purity of the product obtained using the process and (affinity purification) polypeptide of the invention may be determined using any suitable means, such as by the SDS-PAGE method described in WO 2020/115252 (herein incorporated by reference).

In some embodiments, the (affinity purification) polypeptide of the invention in the process defined below results in a product with a yield of at least about 50%, such as about 60%, 70%, 75%, 80% 85% or 90%. The yield of the product obtained using the process and (affinity purification) polypeptide of the invention may be determined using any suitable means.

Thus, a polypeptide of the invention (affinity purification polypeptide) must bind selectively and reversibly to at least one peptide comprising or consisting of an amino acid sequence as set forth in SEQ ID NOs: 3-5 and 17. In a preferred embodiment, the (affinity purification) polypeptide of the invention must bind selectively and reversibly to a peptide comprising or consisting of an amino acid as set forth in SEQ ID NO: 3. Thus, the (affinity purification) polypeptide of the invention binds to at least one peptide comprising or consisting of an amino acid sequence as set forth in SEQ ID NOs: 3-5 or 17 with greater affinity and/or specificity than to other components in the sample (i.e. non-cognate molecules) in which the peptide tag is present. A sample may be any sample (e.g. cell lysate etc. as described below) from which the peptide tag (and associated molecule or component to which the peptide tag is fused or conjugated, i.e. fusion partner) is to be isolated or purified. However, the polypeptide of the invention may also bind to other cognate peptide tags as defined herein.

A non-cognate molecule, particularly a non-cognate peptide or polypeptide may be defined as a peptide or polypeptide that does not contain an amino acid sequence consisting of an amino acid sequence with at least 60% sequence identity to a peptide tag as defined herein, i.e. SEQ ID NOs: 3, 4, 5 or 17. Preferably, the non-cognate molecule does not contain consecutive sequence of 19-23 amino acids with more than about 60%, 55%, 50%, 45%, 40%, 35%, 30%, 25% or 20% sequence identity to a peptide tag as defined herein, i.e. SEQ ID NOs: 3, 4, 5 or 17. Other non-cognate molecules include carbohydrates, sugars, lipids, ions and small molecules.

Suitable conditions for the selective or specific binding of the (affinity purification) polypeptide to its cognate peptide tag may be determined using routine experimentation. For instance, suitable conditions may include the conditions set out above with respect to the peptide tag binding partner polypeptide of the invention and the conditions for the formation of an isopeptide bond with a peptide tag as defined herein.

The term “reversible” or “binds reversibly” refers to the ability of the interaction between the (affinity purification) polypeptide and its cognate peptide tag to be disrupted, resulting in the separation (dissociation) of the complex under suitable conditions. In other words, the non-covalent interaction formed by the affinity purification polypeptide:cognate peptide tag complex can be broken under suitable conditions to enable the separation of the constituent parts. Suitable conditions to dissociate the complex may include any conditions that are able to disrupt or break the non-covalent bonds required to form the complex and may be determined using routine experimentation.

It will be evident that conditions to dissociate the affinity purification polypeptide:cognate peptide tag complex preferably should not lead to irreversible loss of activity of the DogTag peptide and/or fusion partner. For instance, conditions that prevent DogTag from reacting spontaneously with a peptide tag binding partner polypeptide of the invention (e.g. DogCatcher) to form an isopeptide bond should be avoided. Similarly, conditions that alter or inhibit (e.g. denature) the molecule or component fused to the DogTag peptide (i.e. fusion partner, e.g. polypeptide) are not suitable for dissociating the affinity purification polypeptide:cognate peptide tag complex, as such conditions would limit the utility of DogTag fusion in downstream applications. Such conditions will depend on the nature of the fusion partner and the skilled person readily could determine which conditions are suitable (or unsuitable) based on methods known in the art. By way of example, boiling the affinity purification polypeptide:cognate peptide tag complex and/or treatment with 1% sodium dodecyl sulfate (SDS) would dissociate the affinity purification polypeptide:cognate peptide tag complex, but may irreversibly alter (e.g. denature) the fusion partner.

As referred to herein, “functional equivalence” refers to variants of the cognate peptide tag described herein and affinity purification polypeptide of the invention discussed above that may show some reduced selectively (e.g. specificity) or affinity in the binding (formation of the non-covalent complex) with its respective partner (e.g. lower purity or yield in the process of the invention, or activity in a limited range of reaction conditions (e.g. narrower temperature range, such as 10-30° C. etc.)) relative to the parent molecule (i.e. the molecule with which it shows sequence homology), but preferably are as efficient or are more efficient.

A mutant or variant cognate peptide tag described herein with activity that is “equivalent” to the activity of a cognate peptide tag comprising or consisting of an amino acid sequence as set forth in one of SEQ ID NOs: 3-5 or 17 may have activity that is similar (i.e. comparable) to the activity of a peptide tag comprising or consisting of an amino acid sequence as set forth in one of SEQ ID NOs: 3-5 or 17, i.e. such that the practical applications of the peptide tag are not significantly affected, e.g. within a margin of experimental error.

Thus, in some embodiments, an equivalent peptide tag activity means that the mutant or variant cognate peptide tag described is capable of binding selectively and reversibly to the affinity purification polypeptide of the invention. In some preferred embodiments, the mutant or variant cognate peptide tag is capable of spontaneously forming an isopeptide bond with a peptide tag binding partner polypeptide as defined herein with a similar reaction rate (i.e. rate constant as discussed above) and/or yield to a peptide tag comprising or consisting of an amino acid sequence as set forth in one of SEQ ID NOs: 3-5 or 17 under the same conditions.

Similarly, a mutant or variant affinity purification polypeptide of the invention with activity that is “equivalent” to the activity of a polypeptide comprising or consisting of an amino acid sequence as set forth in SEQ ID NO: 18 (preferably wherein X at position 70 is selected from alanine, glycine, serine, asparagine, or threonine) may have activity that is similar (i.e. comparable) to the activity of a polypeptide comprising or consisting of an amino acid sequence as set forth in SEQ ID NO: 18 (preferably wherein X at position 70 is selected from alanine, glycine, serine, asparagine, or threonine), i.e. such that the practical applications of the polypeptide are not significantly affected, e.g. within a margin of experimental error. Thus, an equivalent polypeptide activity means that the mutant or variant affinity purification polypeptide of the invention is capable of binding selectively and reversibly to the cognate peptide tag described herein (e.g. comprising or consisting of an amino acid sequence as set forth in one of SEQ ID NOs: 3-5 or 17) with a similar affinity and/or yield, as described above, to a polypeptide comprising or consisting of an amino acid sequence as set forth in SEQ ID NO: 18 (preferably wherein X at position 70 is selected from alanine, glycine, serine, asparagine, or threonine) under the same conditions.

A mutant or variant polypeptide of the invention with activity that is “equivalent” to the activity of a polypeptide comprising or consisting of an amino acid sequence as set forth in SEQ ID NO: 18 (preferably wherein X at position 70 is selected from alanine, glycine, serine, asparagine, or threonine) may compete with a polypeptide comprising or consisting of an amino acid sequence set forth in SEQ ID NO: 18 (preferably wherein X at position 70 is selected from alanine, glycine, serine, asparagine, or threonine) for binding with a cognate peptide tag as defined herein, e.g. one or all of SEQ ID NOs: 3-5 or 17.

The activity of different polypeptides (e.g. SEQ ID NO: 18 versus mutant) measured under the same reaction conditions, e.g. temperature, ligands (i.e. cognate peptide tag sequence) and their concentration, buffer, salt etc. as exemplified above, can be readily compared to determine whether the affinity and/or yield for each polypeptide is higher, lower or equivalent.

In a particularly useful embodiment, the peptide tag and/or polypeptide (e.g. peptide tag binding partner) is fused or conjugated with another peptide or polypeptide. For instance, the peptide tag and/or polypeptide (e.g. peptide tag binding partner) may be produced as part of another peptide or polypeptide using recombinant techniques as discussed below, i.e. as a recombinant or synthetic protein or polypeptide.

It will be evident that the peptide tag disclosed herein and/or the polypeptide (e.g. peptide tag binding partner) of the invention may be fused to any protein or polypeptide. The protein may be derived or obtained from any suitable source. For instance, the protein may be in vitro translated or purified from biological and clinical samples, e.g. any cell or tissue sample of an organism (eukaryotic, prokaryotic), or any body fluid or preparation derived therefrom, as well as samples such as cell cultures, cell preparations, cell lysates etc. Proteins may be derived or obtained, e.g. purified from environmental samples, e.g. soil and water samples or food samples are also included. The samples may be freshly prepared or they may be prior-treated in any convenient way e.g. for storage.

As noted above, in a preferred embodiment, the peptide or protein fused to the peptide tag disclosed herein and/or polypeptide of the invention may be produced recombinantly and thus the nucleic acid molecules encoding said recombinant proteins may be derived or obtained from any suitable source, e.g. any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa, viruses etc. In some embodiments, the proteins may be synthetic proteins. For example, the peptide and polypeptide (proteins) disclosed herein may be produced by chemical synthesis, such as solid-phase peptide synthesis.

The peptide tag and/or polypeptide (e.g. peptide tag binding partner) may be positioned at any convenient location within a recombinant or synthetic protein. In some embodiments the peptide tag and/or polypeptide (peptide tag binding partner) may be located at the N-terminus or C-terminus of the recombinant or synthetic polypeptide. In some embodiments, the peptide tag and/or polypeptide (e.g. peptide tag binding partner) may be located internally within the recombinant or synthetic polypeptide. Thus, in some embodiments the peptide tag and/or polypeptide (e.g. peptide tag binding partner) may be viewed as an N-terminal, C-terminal or internal domain of the recombinant or synthetic polypeptide.

As noted above, the peptide tag and peptide tag binding partner polypeptide of the present invention are particularly effective in situations where it is necessary to couple proteins together via at least one loop region. Accordingly, in some embodiments, the peptide tag is preferably located internally within the recombinant or synthetic polypeptide. In preferred embodiments, the peptide tag is located within a loop region or domain of the recombinant or synthetic polypeptide. Thus, in some embodiments the peptide tag may be viewed as an internal domain of the recombinant or synthetic polypeptide.

In some embodiments, it may be useful to include one or more spacers, e.g. a peptide spacer, between the peptide or polypeptide to be joined or conjugated with peptide tag and/or polypeptide (e.g. peptide tag binding partner). Thus, the peptide or polypeptide and peptide tag and/or polypeptide (e.g. peptide tag binding partner) may be linked directly to each other or they may be linked indirectly by means of one or more spacer sequences. Thus, a spacer sequence may interspace or separate two or more individual parts of the recombinant or synthetic polypeptide. In some embodiments, a spacer may be N-terminal or C-terminal to the peptide tag and/or polypeptide (e.g. peptide tag binding partner), for example when the peptide tag and/or polypeptide (e.g. peptide tag binding partner) are to be located at the N- or C-terminus of the peptide or polypeptide to be joined or conjugated. As noted above, the peptide tag and polypeptide (e.g. peptide tag binding partner) of the present invention are particularly suitable for use where the peptide tag is located at an internal site in the peptide or polypeptide to be joined or conjugated (or isolated or purified). For example, the peptide tag may be inserted into a loop region. Thus, in some embodiments, spacers may be at both sides of the peptide tag.

The precise nature of the spacer sequence may be of variable length and/or sequence, for example it may have 1-40, more particularly 2-20, 1-15, 1-12, 1-10, 1-8, or 1-6 residues, e.g. 6, 7, 8, 9, 10 or more residues. By way of representative example the spacer sequence, if present, may have 1-15, 1-12, 1-10, 1-8 or 1-6 residues etc. The nature of the residues is not critical and they may for example be any amino acid, e.g. a neutral amino acid, or an aliphatic amino acid, or alternatively they may be hydrophobic, or polar or charged or structure-forming e.g. proline. In some preferred embodiments, the linker is a serine and/or glycine-rich sequence. An exemplary linker/spacer sequence is set forth in SEQ ID NO: 16.

Exemplary spacer sequences thus include any single amino acid residue, e.g. S, G, L, V, P, R, H, M, A or E or a di-, tri- tetra- penta- or hexa-peptide composed of one or more of such residues.

Thus, in some embodiments, the invention provides a recombinant or synthetic polypeptide comprising a polypeptide (e.g. peptide tag binding partner) of the invention as defined above, i.e. a recombinant or synthetic polypeptide comprising a peptide or polypeptide (e.g. a heterologous peptide or polypeptide, i.e. a peptide or polypeptide that is not normally associated with the polypeptide of the invention, e.g. from a different organism) fused to a polypeptide (peptide tag binding partner) of the invention. The recombinant or synthetic polypeptide optionally comprises a spacer as defined above.

The recombinant or synthetic polypeptide of the invention may also comprise purification moieties or tags to facilitate its purification (e.g. prior to use in the methods and uses of the invention discussed below). Any suitable purification moiety or tag may be incorporated into the polypeptide and such moieties are well known in the art. For instance, in some embodiments, the recombinant or synthetic polypeptide may comprise a peptide purification tag or moiety, e.g. a His-tag or C-tag sequence. Such purification moieties or tags may be incorporated at any position within the polypeptide. In some preferred embodiments, the purification moiety is located at or towards (i.e. within 5, 10, 15, 20 amino acids of) the N- or C-terminus of the polypeptide. In some embodiments, the tag may comprise a cysteine residue to facilitate the conjugation of the recombinant or synthetic polypeptide to another molecule or component, e.g. a solid substrate.

As noted above, an advantage of the present invention arises from the fact that the peptide tag and/or polypeptides of the invention incorporated in a peptide or polypeptide (e.g. the recombinant or synthetic polypeptides of the invention) may be completely genetically encoded. Thus, in a further aspect, the invention provides a nucleic acid molecule encoding a polypeptide (e.g. peptide tag binding partner) or recombinant polypeptide as defined above.

In some embodiments, the nucleic acid molecule is codon-optimised for expression in a host cell. Thus, in some embodiments, the nucleic acid molecule is codon optimised for expression in a bacterial cell, such as E. coli, e.g. a nucleotide sequence as set forth in SEQ ID NO: 7. In some embodiments, the nucleic acid molecule is codon optimised for expression in a mammalian cell, such as a human cell, e.g. an HEK cell.

In some embodiments, the nucleic acid molecule encoding a polypeptide binding partner defined above comprises a nucleotide sequence as set forth in SEQ ID NO: 7, or a nucleotide sequence with at least 80% sequence identity to a sequence as set forth in SEQ ID NO: 7.

Preferably, the nucleic acid molecule above is at least 85, 90, 95, 96, 97, 98, 99 or 100% identical to SEQ ID NO: 7.

Nucleic acid sequence identity may be determined by, e.g. FASTA Search using GCG packages, with default values and a variable pamfactor, and gap creation penalty set at 12.0 and gap extension penalty set at 4.0 with a window of 6 nucleotides. Preferably said comparison is made over the full length of the sequence, but may be made over a smaller window of comparison, e.g. less than 300, 200, 100 or 50 contiguous nucleotides.

The nucleic acid molecules of the invention may be made up of ribonucleotides and/or deoxyribonucleotides as well as synthetic residues, e.g. synthetic nucleotides, that are capable of participating in Watson-Crick type or analogous base pair interactions. Preferably, the nucleic acid molecule is DNA or RNA.

The nucleic acid molecules described above may be operatively linked to an expression control sequence, or a recombinant DNA cloning vehicle or vector containing such a recombinant DNA molecule. This allows cellular expression of the peptides and polypeptides of the invention as a gene product, the expression of which is directed by the gene(s) introduced into cells of interest. Gene expression is directed from a promoter active in the cells of interest and may be inserted in any form of linear or circular nucleic acid (e.g. DNA) vector for incorporation in the genome or for independent replication or transient transfection/expression. Suitable transformation or transfection techniques are well described in the literature. Alternatively, the naked nucleic acid (e.g. DNA or RNA, which may include one or more synthetic residues, e.g. base and/or sugar analogues) molecule may be introduced directly into the cell for the production of polypeptides of the invention. Alternatively the nucleic acid may be converted to mRNA by in vitro transcription and the relevant proteins may be generated by in vitro translation.

Appropriate expression vectors include appropriate control sequences such as for example translational (e.g. start and stop codons, ribosomal binding sites) and transcriptional control elements (e.g. promoter-operator regions, termination stop sequences) linked in matching reading frame with the nucleic acid molecules of the invention. Appropriate vectors may include plasmids and viruses (including both bacteriophage and eukaryotic viruses). Suitable viral vectors include baculovirus and also adenovirus, adeno-associated virus, lentivirus, herpes and vaccinia/pox viruses. Many other viral vectors are described in the art. Examples of suitable vectors include bacterial and mammalian expression vectors pGEX-KG, pEF-neo and pEF-HA.

As noted above, the recombinant or synthetic polypeptide of the invention may comprise additional sequences (e.g. peptide/polypeptides tags to facilitate purification of the polypeptide) and thus the nucleic acid molecule may conveniently be fused with DNA encoding an additional peptide or polypeptide, e.g. His-tag, maltose-binding protein etc., to produce a fusion protein on expression.

Thus viewed from a further aspect, the present invention provides a vector, preferably an expression vector, comprising a nucleic acid molecule as defined above.

Other aspects of the invention include methods for preparing recombinant nucleic acid molecules according to the invention, comprising inserting the nucleic acid molecule of the invention encoding the polypeptide (peptide tag binding partner) of the invention into vector nucleic acid.

Nucleic acid molecules of the invention, preferably contained in a vector, may be introduced into a cell by any appropriate means. Suitable transformation or transfection techniques are well described in the literature. Numerous techniques are known and may be used to introduce such vectors into prokaryotic or eukaryotic cells for expression. Preferred host cells for this purpose include insect cell lines, yeast, mammalian cell lines or E. coli, such as strain BL21 (DE3). The invention also extends to transformed or transfected prokaryotic or eukaryotic host cells containing a nucleic acid molecule, particularly a vector as defined above.

Thus, in another aspect, there is provided a recombinant host cell containing a nucleic acid molecule and/or vector as described above.

By “recombinant” is meant that the nucleic acid molecule and/or vector has been introduced into the host cell. The host cell may or may not naturally contain an endogenous copy of the nucleic acid molecule, but it is recombinant in that an exogenous or further endogenous copy of the nucleic acid molecule and/or vector has been introduced.

A further aspect of the invention provides a method of preparing a polypeptide of the invention or recombinant polypeptide as hereinbefore defined, which comprises culturing a host cell containing a nucleic acid molecule as defined above, under conditions whereby said nucleic acid molecule encoding said polypeptide is expressed and recovering said molecule (polypeptide) thus produced. The expressed polypeptide forms a further aspect of the invention.

In some embodiments, the peptide tag disclosed herein and/or polypeptide of the invention, or for use in the method and uses of the invention, may be generated synthetically, e.g. by ligation of amino acids or smaller synthetically generated peptides, or more conveniently by recombinant expression of a nucleic acid molecule encoding said polypeptide as described hereinbefore.

Nucleic acid molecules of the invention may be generated synthetically by any suitable means known in the art.

Thus, the peptide tag disclosed herein and/or polypeptide of the invention may be an isolated, purified, recombinant or synthesised peptide tag or polypeptide.

The term “polypeptide” is used herein interchangeably with the term “protein”. As noted above, the term polypeptide or protein typically includes any amino acid sequence comprising at least 40 consecutive amino acid residues, e.g. at least 50, 60, 70, 80, 90, 100, 150 amino acids, such as 40-1000, 50-900, 60-800, 70-700, 80-600, 90-500, 100-400 amino acids.

Similarly, the nucleic acid molecules of the invention may be an isolated, purified, recombinant or synthesised nucleic acid molecule.

Thus, alternatively viewed, the polypeptides and nucleic acid molecules of the invention preferably are non-native, i.e. non-naturally occurring, molecules.

Standard amino acid nomenclature is used herein. Thus, the full name of an amino acid residue may be used interchangeably with one letter code or three letter abbreviations. For instance, lysine may be substituted with K or Lys, isoleucine may be substituted with I or Ile, and so on. Moreover, the terms aspartate and aspartic acid, and glutamate and glutamic acid are used interchangeably herein and may be replaced with Asp or D, or Glu or E, respectively.

Whilst it is envisaged that the peptide tag disclosed herein and polypeptides of, and for use in, the invention may be produced recombinantly, and this is a preferred embodiment of the invention, it will be evident that the peptide tag disclosed herein and polypeptide of the invention may be conjugated to proteins or other entities, e.g. molecules or components, as defined above by other means. In other words, the peptide tag or polypeptide and other molecule, component or entity, e.g. protein or solid substrate, may be produced separately by any suitable means, e.g. recombinantly, and subsequently conjugated (joined) to form a peptide tag—other component conjugate or polypeptide—other component conjugate that can be used in the methods and uses of the invention. For instance, the peptide tag disclosed herein and/or polypeptide of the invention may be produced synthetically or recombinantly, as described above, and conjugated to another component, e.g. a protein via a non-peptide linker or spacer, e.g. a chemical linker or spacer.

As with the other embodiments discussed above, the peptide tag may be conjugated (joined) to another component at an internal site, i.e. not at one of the termini of the other component. Where the other component is a protein, the peptide tag may preferably be conjugated (joined) to a loop region within said protein.

As discussed above, the affinity purification polypeptide of the present invention forms one part of a two-part affinity purification system and finds particular utility in purifying (i.e. isolating or separating) molecules or components (fusion partners) comprising (e.g. joined or conjugated to) a cognate peptide tag as defined herein.

Thus, in a further aspect, the invention may be seen to provide the use of an affinity purification polypeptide of the invention (e.g. SEQ ID NO: 18) defined above to purify or isolate a molecule or component comprising a cognate peptide tag as defined herein, e.g. a peptide tag having an amino acid sequence with at least 80% sequence identity to a sequence as set forth in one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11.

Affinity purification systems typically utilise a capture molecule (e.g. receptor) immobilised on a solid substrate to facilitate the capture, washing and elution of the target ligand. Thus, the affinity purification polypeptide of the invention may be immobilised (e.g. fused, conjugated or linked) to a solid substrate (i.e. a solid phase or solid support). It will be evident that this may be achieved in any convenient way. Alternatively viewed, the invention provides a solid support on which the polypeptide of the invention is immobilised.

The manner or means of immobilisation and the solid support may be selected, according to choice, from any number of immobilisation means and solid supports as are widely known in the art and described in the literature. Thus, the polypeptide of the invention may be directly bound to the support, for example via a domain or moiety of the polypeptide (e.g. chemically cross-linked). In some embodiments, the polypeptide may be bound indirectly by means of a linker group, or by an intermediary binding group(s) (e.g. by means of a biotin-streptavidin interaction). Thus, the polypeptide may be covalently or non-covalently linked to the solid support. In certain embodiments the polypeptide is immobilised on a solid substrate via a covalent bond.

The linkage may be a reversible (e.g. cleavable) or irreversible linkage. Thus, in some embodiments, the linkage may be cleaved enzymatically, chemically, or with light, e.g. the linkage may be a light-sensitive linkage.

Thus, in some embodiments, the peptide tag and/or polypeptide and other component, e.g. protein or solid substrate, may be joined together either directly through a bond or indirectly through a linking group. Where linking groups are employed, such groups may be chosen to provide for covalent attachment of the peptide tag or polypeptide and other entity, e.g. protein or solid substrate, through the linking group. Linking groups of interest may vary widely depending on the nature of the other entity, e.g. protein. The linking group, when present, is in many embodiments biologically inert.

Many linking groups are known to those of skill in the art and find use in the invention. In representative embodiments, the linking group is generally at least about 50 Daltons, usually at least about 100 Daltons and may be as large as 1000 Daltons or larger, for example up to 1000000 Daltons if the linking group contains a spacer, but generally will not exceed about 500 Daltons and usually will not exceed about 300 Daltons. Generally, such linkers will comprise a spacer group terminated at either end with a reactive functionality capable of covalently bonding to the peptide tag or polypeptide and other molecule or component, e.g. protein or solid substrate.

Spacer groups of interest may include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N, N-diacetic acid, 1,1′-ethylenebis(5-oxo-3-pyrrolidinecarboxylic acid), 4,4′-ethylenedipiperidine, oligoethylene glycol and polyethylene glycol. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the peptide tag/polypeptide binding partner conjugates include heterofunctional compounds, such as azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl-4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like. For instance, a spacer may be formed with an azide reacting with an alkyne or formed with a tetrazine reacting with a trans-cyclooctene or a norbornene.

In some embodiments, it may be useful to modify one or more residues in the peptide tag and/or polypeptide to facilitate the conjugation of these molecules and/or to improve the stability of the peptide tag and/or polypeptide. Thus, in some embodiments, the peptide tag disclosed herein or polypeptide of, or for use in, the invention may comprise unnatural or non-standard amino acids.

In some embodiments, the peptide tag disclosed herein or polypeptide of, or for use in, the invention may comprise one or more, e.g. 1, 2, 3, 4, 5 or more non-conventional amino acids, i.e. amino acids which possess a side chain that is not coded for by the standard genetic code, termed herein “non-coded amino acids”. Such amino acids are well known in the art and may be selected from amino acids which are formed through metabolic processes such as ornithine or taurine, and/or artificially modified amino acids such as 9H-fluoren-9-ylmethoxycarbonyl (Fmoc), (tert)-(B)utyl (o)xy (c)arbonyl (Boc), 2,2,5,7,8-pentamethylchroman-6-sulphonyl (Pmc) protected amino acids, or amino acids having the benzyloxy-carbonyl (Z) group.

Examples of non-standard or structural analogue amino acids which may be used in the peptide tag or polypeptide of, and for use in, the invention are D amino acids, amide isosteres (such as N-methyl amide, retro-inverse amide, thioamide, thioester, phosphonate, ketomethylene, hydroxymethylene, fluorovinyl, (E)-vinyl, methyleneamino, methylenethio or alkane), L-N methylamino acids, D-α methylamino acids, D-N-methylamino acids. Further non-standard amino acids which may be used in the peptide tag disclosed herein and/or polypeptide of, and for use in, the invention are disclosed in Willis and Chin, Nat Chem. 2018; 10(8):831-837, in Table 1 of WO2018/189517 and WO2018/197854, all of which are herein incorporated by reference.

Thus, in some embodiments, a peptide tag or polypeptide of the invention may be provided with means for immobilisation (e.g. an affinity binding partner, e.g. biotin or a hapten, capable of binding to its binding partner, i.e. a cognate binding partner, e.g. streptavidin or an antibody) provided on the support. In some embodiments, the interaction between the peptide tag or polypeptide and the solid support must be robust enough to allow for washing steps, i.e. the interaction between the peptide tag or polypeptide and solid support is not disrupted (significantly disrupted) by the washing steps. For instance, it is preferred that with each washing step, less than 5%, preferably less than 4, 3, 2, 1, 0.5 or 0.1% of the peptide tag or polypeptide is removed or eluted from the solid phase.

The solid support (phase or substrate) may be any of the well-known supports or matrices which are currently widely used or proposed for immobilisation, separation etc. These may take the form of particles (e.g. beads which may be magnetic, para-magnetic or non-magnetic), sheets, gels, filters, membranes, fibres, capillaries, slides, arrays or microtitre strips, tubes, plates or wells etc.

The support may be made of glass, silica, latex or a polymeric material, e.g. a polysaccharide polymer material, such as agarose (e.g. sepharose). Suitable are materials presenting a high surface area for binding of the polypeptide of the invention. Such supports may have an irregular surface and may be for example porous or particulate, e.g. particles, fibres, webs, sinters or sieves. Particulate materials, e.g. beads are useful due to their greater binding capacity, particularly polymeric beads.

Conveniently, a particulate solid support used according to the invention will comprise spherical beads. The size of the beads is not critical, but they may for example be of the order of diameter of at least about 1 μm and preferably at least about 2 μm, 5 μm, 10 μm or 20 μm and have a maximum diameter of preferably not more than about 500 μm, and e.g. not more than about 100 μm.

Monodisperse particles, that is those which are substantially uniform in size (e.g. size having a diameter standard deviation of less than 5%) have the advantage that they provide very uniform reproducibility of reaction. Representative monodisperse polymer particles may be produced by the technique described in U.S. Pat. No. 4,336,173.

However, to aid manipulation and separation, magnetic beads are advantageous. The term “magnetic” as used herein means that the support is capable of having a magnetic moment imparted to it when placed in a magnetic field, and thus is displaceable under the action of that field. In other words, a support comprising magnetic particles may readily be removed by magnetic aggregation, which provides a quick, simple and efficient way of separating the particles following the isopeptide bond formation steps.

In some embodiments, the solid support is a resin, e.g. an amylose resin. In some embodiments, the solid support is a thiol-reactive resin. Thus, in some embodiments, the solid substrate may comprise an iodoacetyl group, e.g. the solid substrate may be an iodoacetyl-activated substrate.

In a further embodiment, the invention provides a kit, particularly a kit for use in the processes and uses of the invention, i.e. for conjugating two molecules or components via an isopeptide bond, wherein two of the molecules or components in the complex are conjugated via an isopeptide bond, wherein said kit comprises:
(a) a peptide tag binding partner polypeptide as defined above, optionally conjugated or fused to a molecule or component, e.g. a protein such as a recombinant or synthetic polypeptide comprising a peptide tag binding partner polypeptide as defined above; and
(b) a peptide (peptide tag) as defined above, optionally conjugated or fused to a molecule or component, e.g. a protein; and/or
(c) a nucleic acid molecule, particularly a vector, encoding a peptide tag binding partner polypeptide as defined in (a); and/or
(d) a nucleic acid molecule, particularly a vector, encoding a peptide tag as defined in (b).

It will be evident that the peptide tag(s) disclosed herein and the peptide tag binding partner polypeptide of the invention have a wide range of utilities. Alternatively viewed, the peptide tag disclosed herein and the peptide tag binding partner polypeptide of the invention may be employed in a variety of industries.

For instance, in some embodiments, the peptide tag(s) disclosed herein and the polypeptide (peptide tag binding partner) of the invention may find utility in targeting fluorescent or other biophysical probes or labels to specific proteins. In this respect, the protein of interest may be modified to incorporate a peptide tag (e.g. one of SEQ ID NOs: 3-5), as discussed above, and the fluorescent or other biophysical probe or label may be fused or conjugated to the polypeptide (peptide tag binding partner, e.g. SEQ ID NO: 1 or 2). The modified protein and probe or label may be contacted together under conditions suitable to allow the spontaneous formation of an isopeptide bond between the peptide tag and polypeptide (peptide tag binding partner), thereby labelling the protein with the label or probe via an isopeptide bond. For instance, the labelled polypeptide of the invention may find utility in an antibody-free Western blot, i.e. where the labelled polypeptide is used to detect a polypeptide containing a DogTag or RrgATag/RrgATag2 peptide (e.g. a peptide having an amino acid sequence as set forth in one of SEQ ID NOs: 3-5) without the need for a separate labelled antibody.

In some embodiments, the peptide tag(s) disclosed herein and polypeptide (peptide tag binding partner) of the invention may find utility in protein immobilisation for proteomics. In this respect, the proteins of interest may be modified to incorporate a peptide tag (e.g. one of SEQ ID NOs: 3-5) and a solid substrate may be fused or conjugated to the polypeptide (peptide tag binding partner, e.g. SEQ ID NO: 1 or 2). The modified proteins and solid substrate may be contacted together under conditions suitable to allow the spontaneous formation of an isopeptide bond between the peptide tag and polypeptide (peptide tag binding partner), thereby immobilising the proteins on the solid substrate via an isopeptide bond. It will be evident that the peptide tag(s) disclosed herein and polypeptide (peptide tag binding partner) of the invention may be used to simultaneously immobilise multiple proteins on a solid phase/substrate, i.e. in a multiplex reaction.

In still further embodiments, the peptide tag(s) disclosed herein and polypeptide (peptide tag binding partner) of the invention may find utility in conjugation of antigens to virus-like particles, viruses, viral vectors, bacteria or multimerisation scaffolds for vaccination. For instance, the production of virus-like particles, viruses, viral vectors or bacteria that display the polypeptide (peptide tag binding partner) of the invention (e.g. SEQ ID NO: 1 or 2) on the surface would facilitate the conjugation of antigens comprising the peptide tag (e.g. one of SEQ ID NOs: 3-5) to their surface via an isopeptide bond. In this respect, antigen multimerisation gives rise to greatly enhanced immune responses. Thus, in some embodiments, the molecule or component fused to the polypeptide of the invention is a viral capsid protein and/or the molecule or component fused to the peptide tag is an antigen, e.g. an antigen associated with a particular disease, e.g. infection, an autoimmune disease, allergy or cancer.

In other embodiments, the peptide tag(s) disclosed herein and polypeptide (peptide tag binding partner) of the invention may be used to cyclise a protein, e.g. an enzyme, e.g. by fusing a peptide tag and binding partner to each end of the protein, e.g. enzyme, and subsequently allowing the spontaneous formation of the isopeptide bond between the peptide tag and polypeptide (peptide tag binding partner). In this respect, cyclisation of enzymes has been shown to increase enzyme resilience.

In particular, cyclisation of enzymes or enzyme polymers (fusion proteins) may improve the thermostability of the protein or protein units in the enzyme polymer. In this respect, enzymes are valuable tools in many processes but are unstable and hard to recover. Enzyme polymers have greater stability to temperature, pH and organic solvents and there is an increased desire to use enzyme polymers in industrial processes. However, enzyme polymer generation commonly uses a glutaraldehyde non-specific reaction and this will damage or denature (i.e. reduce the activity of) many potentially useful enzymes. Site-specific linkage of proteins into chains (polymers) through isopeptide bonds using the peptide tag(s) disclosed herein and polypeptide (peptide tag binding partner) of the present invention is expected to enhance enzyme resilience, such as in diagnostics or enzymes added to animal feed. In particularly preferred embodiments, enzymes may be stabilised by cyclisation, as discussed above.

The peptide tag(s) disclosed herein and polypeptide (peptide tag binding partner) of the invention could also be used to link multiple enzymes into pathways to promote metabolic efficiency, as described in WO 2016/193746. In this respect, enzymes often come together to function in pathways inside cells and traditionally it has been difficult to connect multiple enzymes together outside cells (in vitro). Thus, the peptide tag(s) disclosed herein and polypeptide (peptide tag binding partner) of the invention could be used to couple or conjugate enzymes to produce fusion proteins and therefore enhance activity of multi-step enzyme pathways, which could be useful in a range of industrial conversions and for diagnostics.

The peptide tag(s) disclosed herein and polypeptide (peptide tag binding partner) of the invention will also find utility in the production of antibody polymers. In this respect, antibodies are one of the most important class of pharmaceuticals and are often used attached to surfaces. However, antigen mixing in a sample, and therefore capture of said antigen in said sample, are inefficient near surfaces. By extending chains of antibodies, it is anticipated that capture efficiency will be improved. This will be especially valuable in circulating tumour cell isolation, which at present is one of the most promising ways to enable early cancer diagnosis.

In a still further embodiment, the peptide tag(s) disclosed herein and polypeptide (peptide tag binding partner) of the invention may find utility in the production of drugs for activating cell signalling. In this respect, many of the most effective ways to activate cellular function are through protein ligands. However, in nature a protein ligand will usually not operate alone but with a specific combination of other signalling molecules. Thus, the peptide tag(s) disclosed herein and polypeptide (peptide tag binding partner) of the invention allows the generation of tailored fusion proteins (i.e. protein teams), which could give optimal activation of cellular signalling. These fusion proteins (protein teams) might be applied for controlling cell survival, division, or differentiation.

In yet further embodiments, the peptide tag(s) disclosed herein and polypeptide (peptide tag binding partner) of the invention may find utility in the generation of hydrogels for growth of eukaryotic cells, e.g. neurons, stem cells, preparation of biomaterials, antibody functionalisation with dyes or enzymes and stabilising enzymes by cyclisation.

The primary utility of the affinity purification polypeptide of the invention is in the isolation and/or purification of molecules or components comprising a peptide tag as defined herein. Thus, in a further aspect the invention provides a process for purifying or isolating a molecule or component comprising a peptide (i.e. a cognate peptide tag) having an amino acid sequence with at least 80% sequence identity to a sequence as set forth in one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11, said process comprising:
a) providing a solid substrate on which an affinity purification polypeptide (e.g. SEQ ID NO: 18) of the invention is immobilised;
b) providing a sample comprising said molecule or component;
c) contacting the solid substrate of a) with the sample of b) under conditions that enable said peptide to selectively bind to said polypeptide, thereby forming a non-covalent complex between said polypeptide immobilised on the solid substrate and molecule or component comprising said peptide;
d) washing the solid substrate with a buffer;
e) separating the molecule or component comprising the peptide from the polypeptide immobilised on the solid substrate.

The cognate peptide tag of the affinity purification system described herein may be fused or conjugated to other molecules or to other components or entities (i.e. fusion partners) to facilitate their purification prior to other downstream applications, e.g. reacting the cognate peptide tag with a peptide tag binding partner polypeptides (such as DogCatcher, i.e. a polypeptide comprising an amino acid sequence as set forth in SEQ ID NO: 1). Such molecules or components (i.e. entities) may be a nucleic acid molecule, protein (e.g. an antibody), peptide, lipid, small-molecule organic compound, fluorophore, metal-ligand complex, polysaccharide, nanoparticle, nanotube, polymer, cell, organelle, vesicle, virus, virus-like particle, viral vector or any combination of these.

Thus, process or use of the invention may be used for the purification or isolation of a nucleic acid molecule, protein (e.g. an antibody), peptide, lipid, small-molecule organic compound, fluorophore, metal-ligand complex, polysaccharide, nanoparticle, nanotube, polymer, cell, organelle, vesicle, virus, virus-like particle, viral vector or any combination of these to which the cognate peptide tag is fused or conjugated. Further examples of molecules or components to which the peptide tag may be fused or conjugated are provided above.

The terms “conjugating” or “linking” in the context of the present invention with respect to connecting the cognate peptide tag to molecules or components for purification or isolation in the process or use of the invention refers to joining said peptide tag to said molecules or components, e.g. proteins, via a covalent bond, particularly a peptide bond between the peptide tag and a polypeptide. With respect to connecting the affinity purification polypeptide of the invention to a solid substrate, “conjugating” or “linking” refers to joining said polypeptide to said solid substrate, e.g. beads, via a covalent bond, particularly a thioether bond between the polypeptide (e.g. a cysteine residue in the polypeptide) and solid substrate.

The sample used in the process of the invention (i.e. comprising the molecule or component comprising the cognate peptide tag, e.g. recombinant protein) may be from any biological or clinical sample, e.g. any cell or tissue sample of an organism (eukaryotic, prokaryotic), or any body fluid or preparation derived therefrom, as well as samples such as cell cultures, cell preparations, cell lysates etc. The samples may be freshly prepared or they may be prior-treated in any convenient way e.g. for storage.

In some embodiments, the step of separating the molecule or component comprising the peptide from the polypeptide immobilised on the solid substrate may comprise subjecting the solid substrate to conditions suitable to disrupt the (affinity purification) polypeptide:cognate peptide tag complex, i.e. to disrupt the non-covalent interaction between the polypeptide and the cognate peptide tag. Suitable conditions may depend on the molecule or component linked or conjugated to the polypeptide and may be determined using routine experimentation.

In a representative embodiment, conditions suitable to disrupt the polypeptide:cognate peptide tag complex may comprise contacting said complex with a with a solution comprising imidazole (e.g. at least 1.0 M, e.g. 1.0-4.0 M, 1.0-3.0 M or 2.0-3.0 M, preferably about 2.5 M imidazole). Other conditions that may be suitable to disrupt the complex include contacting the solid substrate with a low pH solution or buffer (e.g. 0.1 M glycine pH 2.0 at 4° C.), subjecting said complex to elevated temperatures, e.g. at least 30, 35, 40 or 45° C., such as 30-65, 35-60, 40-55° C., and/or incubating said complex with a solution comprising a competitor (e.g. a cognate peptide tag as defined above, e.g. SEQ ID NO: 3).

In some embodiments, the solid substrate may be subjected to these conditions repeatedly, e.g. 2, 3, 4, 5 or more times, in order to maximise the yield of the molecule or component to be purified. In some embodiments, it may be advantageous to use a combination of conditions to maximise the yield of the molecule or component to be purified, e.g. a first step using a solution comprising imidazole and a second step using a low pH solution or buffer. Any suitable combination of conditions may be used and is within the purview of the skilled person. In embodiments where competitive peptide elution is used, i.e. wherein the complex is incubated with a competitor, such as the cognate peptide tag, the elution step may be repeated multiple times, e.g. 2, 3, 4, 5 or more times.

A “low pH solution or buffer” may be viewed as any solution or buffer suitable for disrupting the non-covalent interaction between the (affinity purification) polypeptide of the invention and its cognate peptide tag partner. In some embodiments, the low pH solution or buffer is an antibody elution buffer. In this respect, it is evident that the pH of the solution necessary to disrupt the interaction between the (affinity purification) polypeptide of the invention and its cognate peptide tag partner may depend on the components in the solution. By way of example, antibody elution buffers may comprise or consist of 50 mM glycine pH 2.2-2.8 or 100 mM citric acid buffer pH 3.5-4.0. Thus, in some embodiments, the low pH solution or buffer has a pH of 4.0 or less, e.g. 3.9, 3.8, 3.7, 3.6, 3.5, 3.4, 3.3, 3.2, 3.1, 3.0 or less, e.g. about 1.5-3.5, 1.6-3.4, 1.7-3.3, 1.8-3.2, 1.9-3.1 or 2.0-3.0, such as about 2.2-2.8 or 2.5-2.7.

Preferably the conditions that are used to disrupt the (affinity purification) polypeptide:cognate peptide tag complex are such that the cognate peptide tag can still be used in downstream applications, i.e. the conditions do not lead to irreversible loss of activity of the cognate peptide tag.

While the use of peptide tags as defined herein for affinity purification is particularly advantageous because it provides the purified molecule or component with downstream functionality (i.e. the ability to be conjugated to other molecules via a peptide tag binding partner polypeptide of the invention), the process of invention may find utility in the purification or isolation of only the target molecule or component, i.e. without the peptide tag. This may be achieved by separating the target molecule or component from the polypeptide immobilised on the solid substrate through a cleavage reaction that cleaves the peptide tag from the target molecule or component.

Thus, in some embodiments, the step of separating the molecule or component comprising the peptide from the polypeptide immobilised on the solid substrate may comprise subjecting the solid substrate to conditions suitable to cleave the peptide tag from the molecule or component comprising the peptide tag, e.g. by on-resin tag cleavage. This may be accomplished incorporating (e.g. genetically encoding) a cleavage site which can be recognised by one or more proteases specific for that site between the peptide tag and the target molecule or component. Cleavage of the target molecule:peptide tag fusion at the cleavage site by the specific protease(s) releases the target molecule or component from the polypeptide:cognate peptide tag complex, leaving the peptide tag still bound to the polypeptide. Suitable proteases and their respective recognition sites are well known in the art, and any appropriate setup may be utilised in the present method.

Thus, in some embodiments, the molecule or component comprising the peptide tag contains a cleavage site between the peptide tag and molecule or component, e.g. a cleavage site linking the peptide tag and molecule or component. Alternatively viewed, the peptide tag is fused or conjugated to the molecule or component indirectly via a cleavable linker. In some embodiments, the cleavage site or cleavable linker is a protease cleavage site, such as a TEV recognition site. Thus, in some embodiments, the step of separating the molecule or component comprising the peptide from the polypeptide immobilised on the solid substrate may comprise contacting the solid substrate with an entity (e.g. protease, e.g. SuperTEV) under conditions suitable to cleave the cleavage site or cleavable linker thereby releasing the molecule or component from the peptide tag and the polypeptide immobilised on the solid substrate.

The step of washing the solid substrate with a buffer prior to separating said complex from the solid substrate may utilise any suitable buffer, e.g. TBS. The buffer may be selected based on the molecules or components conjugated or linked to the peptide tag. Furthermore, the step of washing the solid substrate may be repeated multiple times, e.g. 2, 3, 4, 5 or more times. Alternatively viewed, in some embodiments the process comprises multiple wash steps, wherein the same or different washing conditions may be used in each step.

Where the solid substrate comprises beads (e.g. agarose-based beads) the volume of buffer used in the wash steps may be at least about 2 times the volume of the beads, e.g. at least about 3, 4, 5, 6, 7, 8, 9 or 10 times the volume of the beads.

In some embodiments, the solid substrate is subjected to stringent washing conditions. The nature of the stringent washing conditions will depend on the molecules or components conjugated or linked to the peptide tags and/or the composition of the solid substrate. The skilled person could select such conditions as a matter of routine.

The temperature of the washing and separation (elution) steps may be determined readily by a person of skill in the art based on routine experimentation and may depend on the nature of the molecule or component being isolated or purified. In some embodiments, the washing and/or separation steps are performed at 10° C. or less, e.g. 9, 8, 7, 6, 5 or 4° C. or less.

Whilst it may be useful to immobilise the affinity purification polypeptide of the invention on a solid support prior to contact with the sample comprising the molecule or component comprising the cognate peptide tag, it will be evident that this is not essential. For instance, the binding of the polypeptide of the invention and the component comprising the cognate peptide tag may take place in solution, which is subsequently applied to a solid support or solid phase, e.g. column, for subsequent washing and separation (e.g. elution) steps. In some embodiments, the polypeptide:cognate peptide tag complex may be applied to the solid phase under conditions suitable to immobilise the complex on the solid phase via the polypeptide (e.g. an immobilisation domain on the polypeptide), washed under suitable conditions and subsequently subjected to one or more of the conditions mentioned above, e.g. contacted with a solution comprising imidazole, to disrupt the complex, thereby separating the polypeptide and the component comprising the cognate peptide tag.

In a further aspect, the invention provides an apparatus for use in the process or use hereinbefore defined comprising a solid substrate on which the (affinity purification) polypeptide of the invention is immobilised.

In some embodiments, the apparatus may comprise a chromatography column comprising the solid substrate on which the (affinity purification) polypeptide of the invention is immobilised. The apparatus may further comprise means for contacting the solid substrate with the sample, washing and elution buffers and/or means for removing (e.g. aspirating) or collecting liquids (e.g. wash-through, eluted fractions) from the solid substrate.

In a further aspect, the invention provides a kit, particularly a kit for use in preparing a solid substrate on which the (affinity purification) polypeptide of the invention is immobilised, comprising:
a) the (affinity purification) polypeptide of the invention; and
b) means for immobilising the polypeptide of a) on a solid substrate.

In a further embodiment, the kit further comprises a solid substrate as defined above.

Means for immobilising the polypeptide of the invention on a solid substrate may comprise reagents for activating the solid substrate (e.g. resin) and/or polypeptide (e.g. tris(2-carboxyethyl)phosphine), reagents for coupling the polypeptide to the solid substrate (e.g. coupling buffer, such as 50 mM Tris-HCl, 5 mM EDTA, pH 8.5) and/or reagents for blocking the solid substrate (e.g. L-cysteine-HCl in coupling buffer).

The invention will now be described in more detail in the following non-limiting Examples with reference to the following drawings:

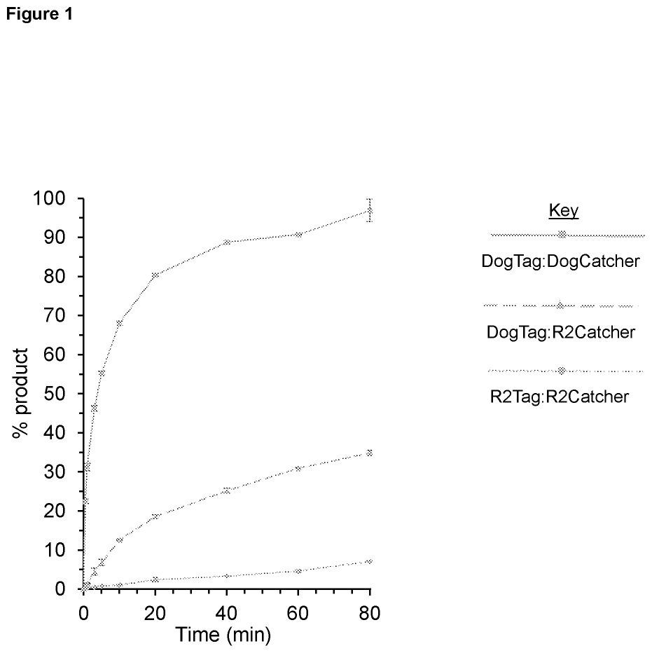

FIG. 1 Amide bond formation rate for R2Tag/R2Catcher, with the increase upon use of DogTag (DogTag/R2Catcher curve) and upon use of DogCatcher (DogTag/DogCatcher curve) was measured in PBS pH 7.5 at 25° ° C. with 5 μM of each protein. Mean±1 s.d., n=3 based on SDS-PAGE densitometry. Some error bars are too small to be visible.

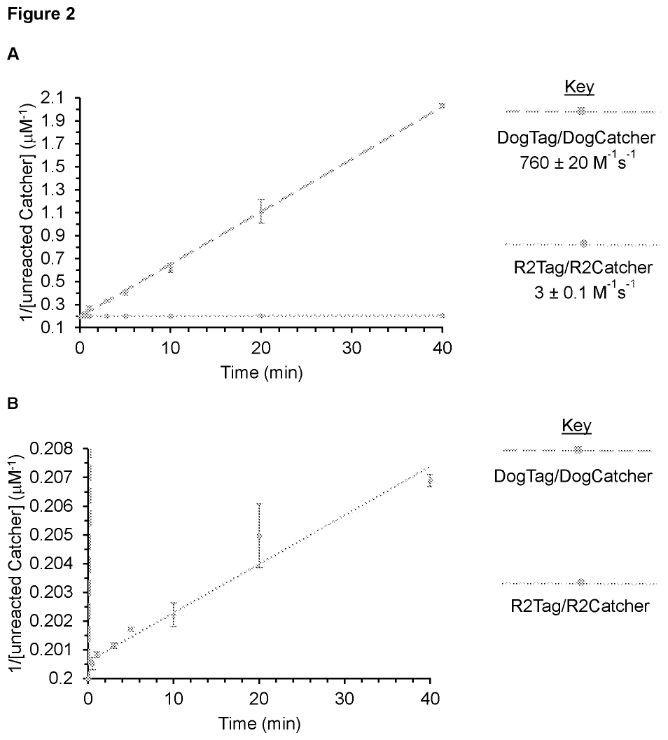

FIG. 2 Second-order rate constant determination for DogTag/DogCatcher and R2Tag/R2Catcher. (A) Time-course of reaction for DogTag/DogCatcher or R2Tag/R2Catcher. 5 μM AviTag-DogTag-MBP and 5 μM DogCatcher or 5 μM AviTag-R2Tag-MBP and 5 μM R2Catcher were incubated in PBS pH 7.5 at 25° C., with quantification by SDS-PAGE/Coomassie and densitometry (mean±1 s.d., n=3). Some error bars are too small to be visible. The resultant second-order rate constant is marked (mean±1 s.d., n=3). (B) Zoom of the y-axis from (A), to make the data clearer for R2Tag/R2Catcher.

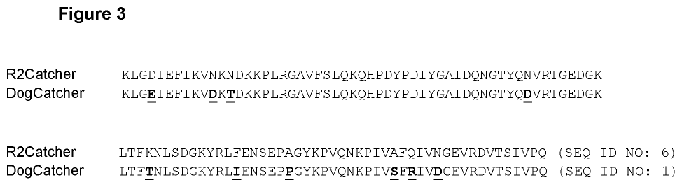

FIG. 3 Sequence alignment of R2Catcher (SEQ ID NO: 6) with DogCatcher (SEQ ID NO: 1). The mutations to create DogCatcher are underlined and in bold.

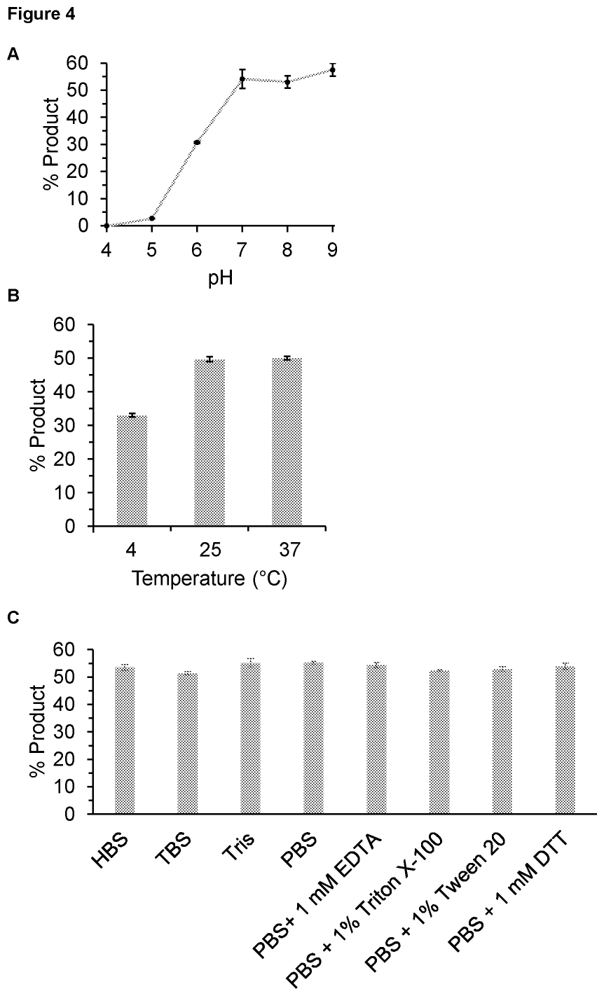

FIG. 4 Condition-dependence of DogTag/DogCatcher reactivity. (A) pH-dependence. 2 μM AviTag-DogTag-MBP and 2 μM DogCatcher were reacted for 30 min at 25° C. in SPG buffer at the indicated pH. (B) Temperature-dependence. 2 μM AviTag-DogTag-MBP and 2 μM DogCatcher were reacted for 30 min at 25° C. in SPG pH 7.0 at the indicated temperature. (C) Buffer-dependence. 5 μM AviTag-DogTag-MBP and 5 μM DogCatcher were reacted for 5 min at 25° C. at pH 7.5 in the indicated buffer (HBS is HEPES-buffered saline; TBS is Tris-buffered saline). Data represent mean±1 s.d., n=3; some error bars are too small to be visible.

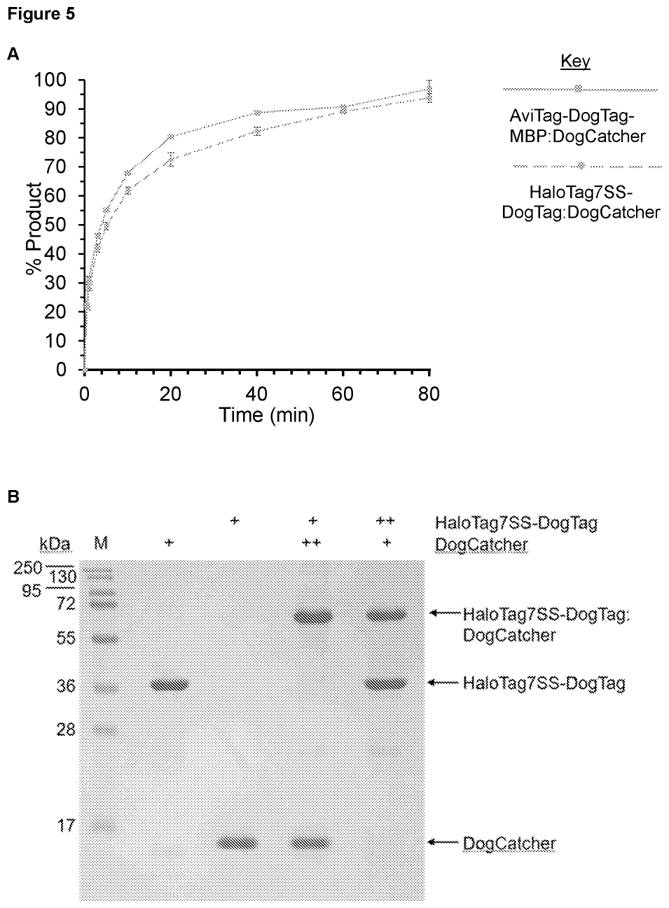

FIG. 5 DogTag/DogCatcher reaction to completion when DogTag was internal. (A) DogCatcher reaction rate with the internal DogTag in HaloTag7SS was similar to that for the unconstrained DogTag in AviTag-DogTag-MBP. Data represent mean±1 s.d., n=3; some error bars are too small to be visible. (B) Testing DogTag/DogCatcher reaction to completion. DogCatcher was incubated with HaloTag7SS-DogTag in PBS pH 7.5 for 200 min, before SDS-PAGE with Coomassie staining. +=10 μM, ++=20 μM. M=molecular weight markers. 98% loss was seen for HaloTag7SS-DogTag in the presence of excess DogCatcher, based on densitometry. 98% loss was seen for DogCatcher in the presence of excess HaloTag7SS-DogTag.

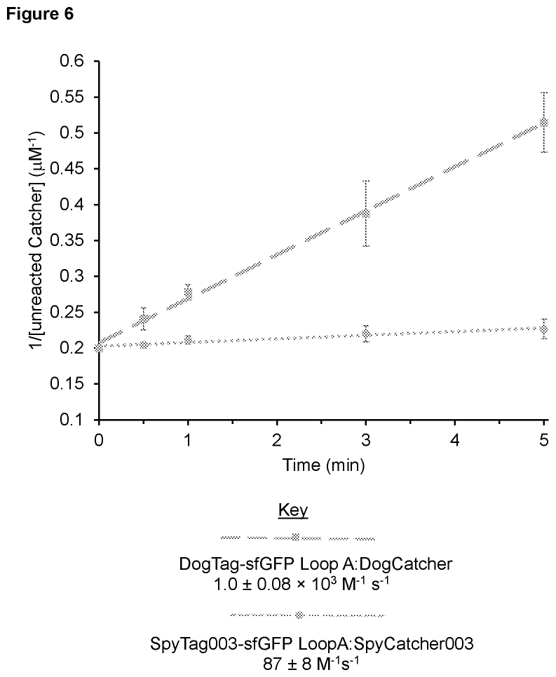

FIG. 6 DogTag functioned well within the β-barrel domain of sfGFP and reacted faster than SpyTag003. Second-order reaction plot comparing the reaction speed of DogCatcher with DogTag in sfGFP Loop A relative to SpyCatcher003 reaction with SpyTag003. Mean±1 s.d., n=3. Some error bars are too small to be visible.

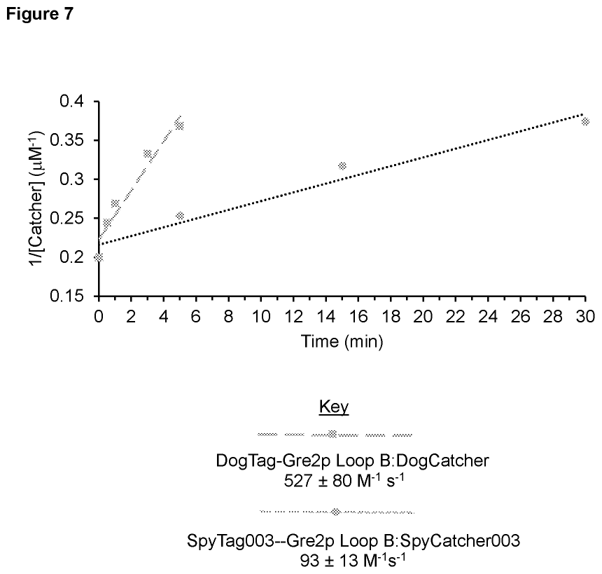

FIG. 7 Tag reactivity and enzyme activity after Tag insertion in loops of isovaleraldehyde reductase. Second-order reaction plot. DogTag/DogCatcher reacted faster than SpyTag003/SpyCatcher003 in loop B of Gre2p.

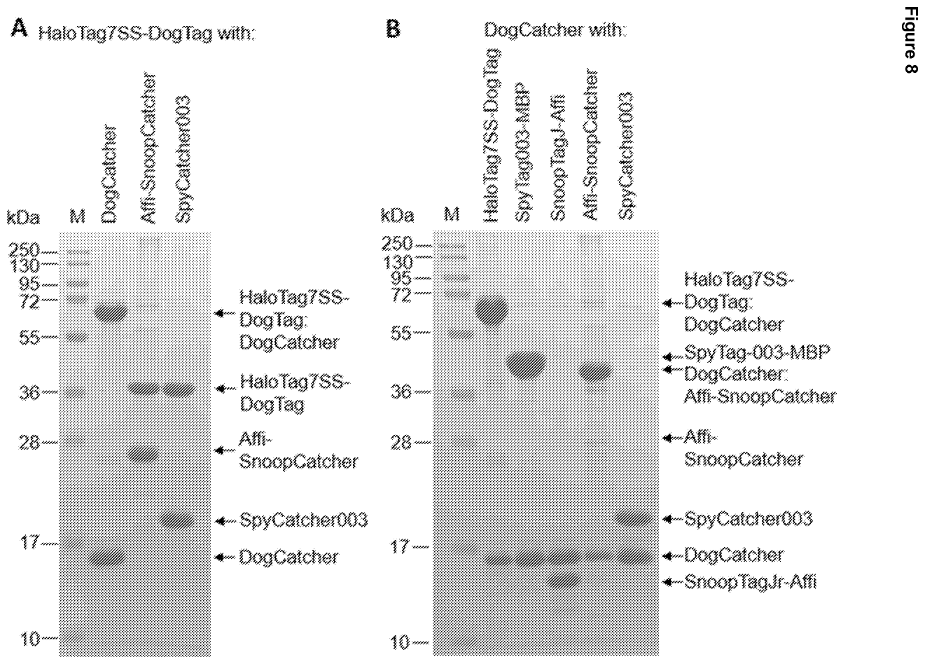

FIG. 8 DogTag/DogCatcher orthogonality. (A) DogTag reacted with DogCatcher but not SnoopCatcher or SpyCatcher003. 15 μM DogCatcher, Affi-SnoopCatcher or SpyCatcher003 was incubated with 10 μM HaloTag7SS-DogTag for 24 h in PBS pH 7.5 at 25° C., before SDS-PAGE with Coomassie staining. (B) DogCatcher reacted with DogTag and SnoopCatcher. 15 μM DogCatcher was incubated with 10 μM HaloTag7SS-DogTag, SpyTag003-MBP, SnoopTagJr-Affi, Affi-SnoopCatcher or SpyCatcher003 for 24 h in PBS pH 7.5 at 25° C., before SDS-PAGE with Coomassie staining. M=molecular weight markers.

FIG. 9 The bar chart shows the effects of various modifications to R2Catcher on its solubility, based on the yield of soluble protein from 1 L culture of E. coli after Ni-NTA purification.

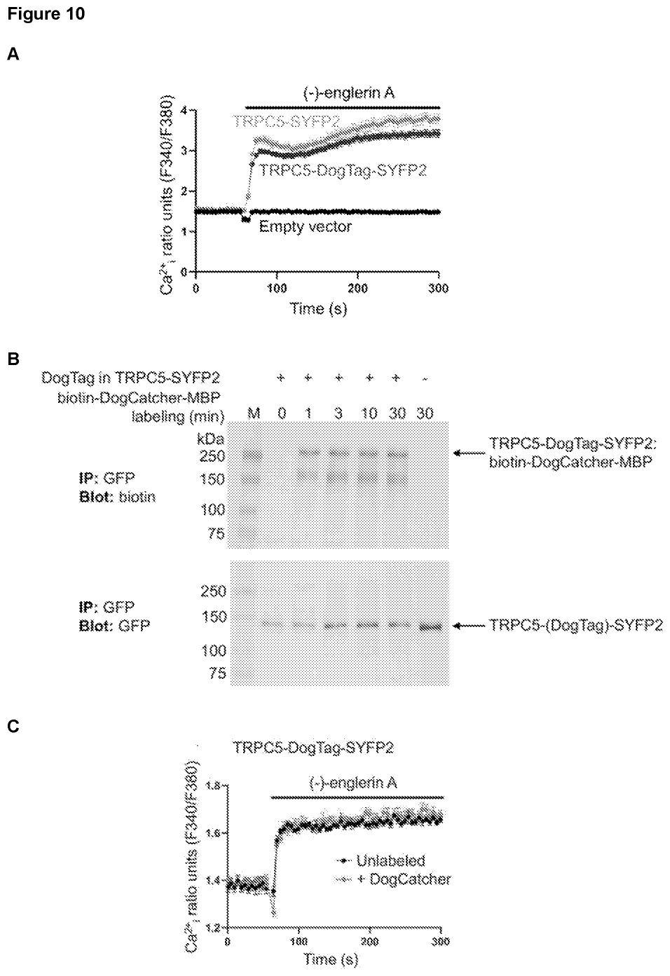

FIG. 10 shows the results of specific targeting of an ion channel at the mammalian cell-surface using DogTag/DogCatcher. (A) DogTag insertion had minimal effect on ion channel opening. Representative intracellular calcium measurements (Ca²⁺ᵢ) from one 96-well plate (mean±1 SE, n=4) showing activation of TRPC5-SYFP2 (grey trace) or TRPC5-DogTag-SYFP2 (middle trace) in HEK 293 cells by 30 nM (−)-englerin A (present during the period marked with a horizontal line). No calcium response was induced by (−)-englerin A in empty vector-transfected cells (lower trace). (B) Rapid labelling by DogCatcher at the cell surface. COS-7 cells expressing TRPC5-DogTag-SYFP2 or TRPC5-SYFP2 control were incubated with 5 μM biotin-DogCatcher-MBP for the indicated time at 25° C. Cell lysates were immunoprecipitated with GFP-Trap before blotting for either biotin (top panel) or fluorescent protein (bottom panel). (C) DogCatcher reaction had minimal effect on ion channel opening. Representative intracellular calcium measurements (Ca²⁺ᵢ) from one 96-well plate (mean±1 SE, n=6) showing activation of TRPC5-DogTag-SYFP2 in HEK293 cells by 10 nM (−)-englerin A (present during the period marked with a horizontal line), with (grey trace) or without (black trace) 30 min pre-treatment with 5 μM biotin-DogCatcher-MBP.

### EXAMPLES

### Example 1: Improvement of a Tag-Catcher Pair Derived from RrgA Domain 4

RrgA is an adhesin from S. pneumoniae that consists of 4 domains. Domain 4 (residues 734-861) forms a spontaneous intramolecular isopeptide bond by a transamidation reaction between Lys742 and Asn854, directed by Glu803. This domain was previously split and engineered to create the protein coupling reagents R2Catcher (also known as RrgACatcher (SEQ ID NO: 6), corresponding to residues 734-838 of RrgA and containing the reactive Lys and catalytic Glu) and R2Tag (SEQ ID NO: 17, which corresponds to residues 838-860 of RrgA).

It was found that R2Tag and R2Catcher did successfully reconstitute and react upon mixing, but the rate was slow (FIG. 1). The second-order rate constant was determined as 3±0.1 M⁻¹s⁻¹ (mean±1 s.d., n=3) in PBS pH 7.4 at 25° C. (FIG. 2). R2Tag was engineered for faster reconstitution. The flexible Gly at 842 within a β-strand was substituted with Thr, maintaining hydrophilicity and being favoured within β-sheets. Asp848 was substituted with Gly to favour tight turn formation. Asn847 was substituted with Asp to improve electrostatic interaction with Lys 849. R2Tag with the mutations G842T, N847D, and D848G (termed DogTag, SEQ ID NO: 3) improved reaction 10-fold with R2Catcher. The second-order rate constant for DogTag with R2Catcher was 30±2 M⁻¹s⁻¹ (mean±1 s.d., n=3).

A major problem for R2Catcher was its limited solubility in PBS pH 7.4 (˜140 μM), which is low when compared to SpyCatcher (>1 mM). SnoopLigase, a polypeptide derived from the D4 domain of RrgA, has previously been optimised computationally via PROSS and Rosetta, leading to mutations D737S, D838G, and I839V. However, mutation of acidic residues in R2Catcher variants led to highly insoluble proteins at neutral pH. The inventors observed that the predicted pI of R2Catcher was close to neutral (6.6) and hypothesised that the introduction of mutations to increase the surface negative change of R2Catcher may improve the solubility of the protein. The inventors identified numerous mutations that may increase the surface negative change of the polypeptide. Selected mutations were evaluated by Rosetta to see that the mutation did not greatly reduce the predicted stability of the polypeptide (see Table 1).

TABLE 1
Predicted stability changes for mutations in R2Catcher. Protein
stabilities are calculated by Rosetta as the difference
in the relative energy units (DREU) for the isopeptide bond-
formed version relative to R2Catcher isopeptide

| | ΔREU_{isopeptide} |
|---|---|
| Protein model | (kcal · mol⁻¹) |
| PDB: 2WW8 734-860 energy-minimised against | 0 |
| CCP4 map (R2Catcher) | |
| R2Catcher + A808P | −5.0 |
| R2Catcher + N744D N746T A808P | −4.8 |
| R2Catcher + D737E K792T A808P | −6.9 |
| R2Catcher + D737E N744D N746T K792T | −6.6 |
| R2Catcher + A808P N780D | −5.0 |
| R2Catcher + A808P N825D | −4.2 |
| R2Catcher + N780D A808P N825D | −7.5 |
| R2Catcher + D737E N744D N746T N780D | −5.4 |
| A808P K792T N825D (R2CatcherB) | |
| R2CatcherB + F802I | −6.9 |
| R2CatcherB + Q822R | −6.2 |
| R2CatcherB + A820S | −5.5 |
| R2CatcherB + F802I A820S Q822R | −11.4 |
| (DogCatcher) | |

The combination of mutations D737E, N744D, N746T, N780D, K792T, and N825D, in addition to A808P, which was introduced to reduce the conformational flexibility of a β-turn in R2Catcher increased the solubility to 316 μM in PBS PH 7.4. The resultant mutant was terms R2CatcherB (SEQ ID NO: 8).

### Example 2: Improvement of R2Catcher Reactivity

Phage display of new protein scaffolds often runs into obstacles, including misfolding, degradation in the periplasm, loss of phage infectivity, and accumulation of frame-shifted or truncated variants. Therefore, it was necessary to optimise R2Catcher rationally, before attempting directed evolution. With the highly soluble R2CatcherB in hand, the inventors applied directed evolution to enhance reaction speed. A library of mutations in R2CatcherB was generated by error-prone PCR. During conventional phage display panning, non-covalently bound phage are eluted from the bait protein by conditions such as glycine pH 2.5. In the current approach, this same wash was used to remove any non-covalently bound phage, to select only for variants that allow isopeptide bond formation to occur. Phage were then specifically eluted using TEV protease. Following multiple rounds of phage display and evaluation of different phage libraries, the best performing variant, termed DogCatcher (SEQ ID NO: 1), reacted with AviTag-DogTag-MBP 25-fold faster than R2Catcher (760±20 M⁻¹s⁻¹, mean±1 s.d., n=3) (FIGS. 1 and 2). DogCatcher contained 3 further mutations compared to R2CatcherB (F802I, A820S, and Q822R) (FIG. 3). The effect of these mutations on domain stability was assessed individually using Rosetta and found only a minor predicted change (Table 1 above). Overall, DogTag/DogCatcher represents a 250-fold improvement of the rate of reaction over the initial split pair (R2Tag and R2Catcher) (FIGS. 1 and 2).

### Example 3: Characterisation of DogTag/DogCatcher

The DogTag/DogCatcher pair was characterised to determine its dependence on reaction conditions (FIG. 4).

DogTag/DogCatcher reacted poorly at pH 4 and 5, with reactivity rising sharply to pH 7 and high reactivity maintained at pH 8 and 9 (FIG. 4A). DogTag/DogCatcher was shown to have substantial activity at 4° C., along with high reactivity from 25-37° C. (FIG. 4B). DogTag/DogCatcher showed high reactivity in a range of buffers (HEPES, PBS, Tris) and was tolerant to chelator (EDTA) or detergent (FIG. 4C).

### Example 4: DogTag Inserted within a Loop Retained Good DogCatcher Reactivity

The Tag/Catcher approach has been employed on hundreds of proteins, with the vast majority inserting the Tag at a flexible terminus of the protein of interest. Given that DogTag is expected to form a β-hairpin to reconstitute the Domain 4 structure, the inventors hypothesised that constraining DogTag at a structured internal site of a protein would allow efficient isopeptide bond formation. Therefore, the inventors assayed DogTag inserted in an α-helix in the 42 kDa HaloTag7 protein (a version named HaloTag7SS) between residues 139 and 140. Comparison with reaction of a non-constrained DogTag (fused N-terminally to the MBP domain) revealed that DogTag demonstrated similar reactivity in these different environments (FIG. 5A).

The ability of DogTag/DogCatcher reaction to go to completion was also tested. With two-fold excess of DogCatcher, 98% of HaloTag7SS-DogTag reacted (FIG. 5B). Conversely, with two-fold excess of HaloTag7SS-DogTag, 98% of DogCatcher reacted (FIG. 5B).

### Example 5: DogTag was Superior to SpyTag003 for Catcher Reactivity within Superfolder GFP

The insertion of a Tag such as SpyTag003 or DogTag into the loop within the protein should ideally allow both high reactivity with the Catcher protein, as well as retaining the function of the host protein. In the first case, DogTag or SpyTag003 flanked on each side by G₅S linkers was cloned into loops within superfolder GFP (sfGFP), a β-barrel protein previously shown permissible for loop insertions. All the variants of sfGFP were solubly expressed (with DogTag or SpyTag003 and loops A, B or C).

A major difference in reactivity was observed between the Catchers. For reaction of DogTag within Loop A with DogCatcher (FIG. 6), the second-order rate constant was 1.0±0.08×10³ M⁻¹s⁻¹ (mean±1 s.d., n=3), which is comparable to the rate for a terminal DogTag fusion (FIG. 2). In contrast, the second-order rate constant for SpyCatcher003 reaction with SpyTag003 in the same loop of sfGFP is 87±8 M⁻¹s⁻¹ (mean±1 s.d., n=3), 6,000-fold slower than for SpyTag003 as a terminal fusion (5.5±0.6×10⁵ M⁻¹s⁻¹).

All the loop-insertion variants of sfGFP showed comparable absorption intensity and spectrum to unfused WT sfGFP. Similarly, there was minimal change to the intensity or spectrum of fluorescence emission for any of the variants. Therefore, insertion of DogTag or SpyTag003 was well tolerated for retention of fluorescent protein function.

### Example 6: DogTag could be Inserted into Loops within an Enzyme Whilst Maintaining Catalytic Activity

Tag/Catcher reaction has been used for scaffolding of multi-enzyme complexes and creation of catalytic hydrogels. The isovaleraldehyde reductase Gre2p was used with SpyTag/SpyCatcher in this application and has a mixed β-α-β Rossmann fold. This protein was selected to test whether DogTag/DogCatcher can be used in an enzyme which must maintain flexibility for efficient function. Three loops within Gre2p away from the active site were selected to insert DogTag or SpyTag003 flanked by G₅S linkers. All the insertions of SpyTag003 or DogTag allowed soluble enzyme expression. Reduction of isovaleraldehyde to isoamyl alcohol by Gre2p is NADPH-dependent. The absorbance change upon NADPH oxidation into NADP⁺ was used to follow the reaction of wild-type (WT) and loop-inserted Gre2p variants. With SpyTag003 or DogTag in each loop, the isovaleraldehyde reductase activity was successfully maintained within 2-fold of WT Gre2p (Table 2).

TABLE 2
Specific enzyme activities for Gre2p variants. Each Gre2p variant
was incubated with isovaleraldehyde and NADPH in phosphate
buffer at 25° C. and reaction was monitored spectrophotometrically
(mean ± 1 s.d., n = 3 biological replicates).

| | Specific activity |
|---|---|
| Gre2p Variant | (μmol_{NAPDH} · min⁻¹ · μmolₚᵣₒₜₑᵢₙ⁻¹) |
| WT | 1,892 ± 294 |
| SpyTag003 Loop A | 2,391 ± 347 |
| SpyTag003 Loop B | 1,572 ± 372 |
| SpyTag003 Loop C | 1,814 ± 83 |
| DogTag Loop A | 3,087 ± 259 |
| DogTag Loop B | 3,268 ± 361 |
| DogTag Loop C | 1,484 ± 223 |

For Gre2p loop B, the second-order rate constant for reaction of DogTag with DogCatcher was 527±80 M⁻¹s⁻¹, whilst reaction was much slower for SpyTag003 with SpyCatcher003 (93±13 M⁻¹s⁻¹; mean±1 s.d., n=3, FIG. 7).

### Example 7: DogTag/DogCatcher Orthogonality Testing

SnoopTagJr/SnoopCatcher is derived from the D4 domain of RrgA and is orthogonal to the SpyTag/SpyCatcher family of Tag/Catchers. The cross-reactivity of DogTag/DogCatcher with SnoopTagJr/SnoopCatcher or SpyTag003/SpyCatcher003 was tested. DogTag only reacted with DogCatcher (FIG. 8A), even after 24 h at high protein concentrations. DogCatcher only reacted with DogTag-containing Tag/Catcher constructs (FIG. 8B). Consequently, DogCatcher did not react with SpyTag003, SpyCatcher003 or SnoopTagJr. In contrast, DogCatcher reacted to completion with HaloTag7SS-DogTag or SnoopCatcher (FIG. 8B). DogCatcher reacts with SnoopCatcher because SnoopCatcher contains a sequence like DogTag at its C-terminus (with DogCatcher likewise containing a sequence like SnoopTag at its N-terminus).

### Example 8: DogCatcher Reacts Specifically with an Ion Channel at the Mammalian Cell Surface

Various cell-surface proteins lack N or C termini accessible at the plasma membrane. Therefore, covalent labelling with exogenous probes could be facilitated by loop-mediated ligation.

Transient receptor potential canonical 5 (TRPC5) is an ion channel permeable to Na⁺ and Ca²⁺ and involved in various conditions, including anxiety, kidney disease, and cardiovascular and metabolic disease. Both termini of TRPC5 are on the cytosolic side of the membrane.

DogTag was genetically inserted into the second extracellular loop of TRPC5 between residues 460 and 461, at a site distant from the pore. The bright and rapidly maturing yellow fluorescent protein SYFP2 was fused to the C-terminus, which allows imaging of the distribution of total TRPC5 but does not highlight the active surface pool.

Intracellular calcium measurements in transiently transfected HEK293 cells were performed to test the functionality of the DogTag insertion by stimulating TRPC5 opening with the sesquiterpenoid activator (−)-englerin A. The DogTag fusion formed functional channels with efficient agonist response (FIG. 10A).

The efficacy of DogCatcher recognition at the cell surface was tested by adding biotin-DogCatcher-MBP to COS-7 cells expressing TRPC5-DogTag-SYFP2. Whole-cell lysate was blotted with streptavidin-HRP, after GFP-Trap pull-down of the SYFP2 fusion. There was rapid reaction of DogCatcher with TRPC5-DogTag-SYFP2, detectable after only 1 min incubation, with minimal signal on the negative control cells lacking DogTag fusion (FIG. 10B).

The functionality of TRPC5 in HEK293 cells after labelling with biotin-DogCatcher-MBP was also tested. DogCatcher labelling had no effect on TRPC5-mediated calcium influx into these cells stimulated by (−)-englerin A (FIG. 10C).

To visualize the surface exposed TRPC5 pool, a unique cysteine was introduced the N-terminus of DogCatcher and coupled to maleimide-Alexa Fluor 647, to give DogCatcher-647. DogCatcher-647 allowed selective staining of TRPC5-DogTag-SYFP2 in COS-7 cells, compared with the controls lacking DogTag, with receptor visualization by confocal fluorescence microscopy.

DogCatcher staining was observed as early as 1 min after addition, with optimal staining at 10 min. Overall, DogTag/DogCatcher allowed rapid and selective covalent labelling of an ion channel at the surface of different mammalian cell types.

### Conclusion

The DogTag/DogCatcher pair is efficient for covalent protein-protein reaction in diverse protein loops. DogTag/DogCatcher shows a number of features that make the system easy to apply. Both partners are genetically encodable from the regular 20 amino acids, with reaction tolerant to a range of conditions (4-37° C., pH 6-8, detergents, and different buffers). Reaction can proceed to ˜98% conversion without detectable side-products and leaves an amide bond which is anticipated to have high stability. Neither DogTag nor DogCatcher contains any cysteine residues, so coupling can be performed on proteins requiring reducing or oxidizing conditions.

DogTag reacts efficiently with DogCatcher at the terminus of a protein or inserted internally in proteins that are predominantly α-helical, predominantly β-sheet, or α+β folds. Maintenance of good fluorescence characteristics when inserted in different loops of sfGFP, and good catalytic activity in different loops of Gre2p, was observed. Insertion of DogTag within a loop of a membrane protein (TRCP5) also enabled labelling of mammalian cells. In the case of HaloTag, DogTag was inserted within a secondary structure element.

It is a considerable challenge to obtain Tag/Catcher pairs with rapid and high yielding reaction. The majority of Tag/Catcher pairs in the literature require high micromolar concentration and days for substantial coupling. In some cases, the split proteins show no reactivity at all. Therefore, substantial protein engineering effort was required to achieve efficient spontaneous intermolecular isopeptide bond formation demonstrated herein. The rate of DogTag/DogCatcher reaction was comparable at a terminal site or a loop site and the DogTag/DogCatcher pair therefore represents a preferred pairing for reaction with various loops.

### Materials and Methods

### Plasmids and Cloning of Constructs

PCR-based cloning and site-directed mutagenesis were carried out by standard methods using Q5 High-Fidelity Polymerase (NEB) or KOD polymerase (EMD Millipore) and Gibson assembly. pDEST14-R2Catcher was derived by cloning residues 734-838 of the RrgA adhesin from Streptococcus pneumoniae TIGR4 (GenBank AAK74622), with numbering based on PDB ID 2WW8 into the backbone from pDEST 14-SpyCatcher (GenBank JQ478411, Addgene plasmid ID 35044). Mutations D737E, N744D, N746T, N780D, K792T, A808P and N825D were overlaid on to R2Catcher to form pDEST14-R2CatcherB by Gibson assembly. Phagemid vector pFab5cHis-R2CatcherB was derived from pFab5cHis-SpyCatcher-gIII. pDEST14-DogCatcher (FIG. 3) was derived from pDEST14-R2CatcherB by inclusion of the F802I, A820S and Q822R mutations by Gibson assembly. pDEST14-SpyCatcher003 has been described (GenBank Accession no. MN433887, Addgene plasmid ID 133447). pET28-AviTag-R2Tag-MBP was derived from pET28a-SpyTag003-MBP (GenBank Accession no. MN433888, Addgene plasmid ID 133450). pET28-AviTag-DogTag-MBP was derived from pET28a-SpyTag003-MBP (GenBank Accession no. MN433888, Addgene plasmid ID 133450). pET28-AviTag-DogTag NA-MBP was derived from pET28-AviTag-DogTag-MBP by Gibson assembly. pET28a-HaloTag7SS-DogTag encodes DogTag inserted in HaloTag7 between residues D139 and E140 and C61S and C261S mutations in HaloTag7 to block disulfide bond formation. pET28-Gre2p was derived from pET28-SpyTag003-sfGFP (Addgene plasmid ID 133454) by inserting the Gre2p isovaleraldehyde reductase from Saccharomyces cerevisiae (as a synthetic gene block with codons optimised for expression in E. coli B strains) in place of sfGFP by Gibson assembly. pET28-Gre2p-SpyTag003 loop insertions were derived from pET28-Gre2p by insertion of spacer-SpyTag003-spacer (sequence GGGGSRGVPHIVMVDAYKRYKGGGGS, SEQ ID NO: 10) between residues Lys140 and Ser141 (pET28-Gre2p-SpyTag003 Loop A), Glu229 and Asp230 (pET28-Gre2p-SpyTag003 Loop B), or Ser297 and Thr303 (pET28-Gre2p-SpyTag003 Loop C) by Gibson assembly. pET28-Gre2p-DogTag loop insertions were derived from pET28-Gre2p by insertion of spacer-DogTag-spacer (sequence GGGGSDIPATYEFTDGKHYITNEPIPPKGGGGS, SEQ ID NO: 11) between residues Lys 140 and Ser141 (pET28-Gre2p-DogTag Loop A), Glu229 and Asp230 (pET28-Gre2p-DogTag Loop B), or Ser297 and Thr303 (pET28-Gre2p-DogTag Loop C) by Gibson assembly. pET28-sfGFP was derived from pET28-SpyTag003-sfGFP (Addgene plasmid ID 133454) by deletion of the N-terminal SpyTag003 by Gibson assembly. pET28-sfGFP-SpyTag003 loop insertions were derived from pET28-sfGFP by insertion of spacer-SpyTag003-spacer (SEQ ID NO: 10) between residues Val22 and Asn23 (pET28-sfGFP-SpyTag003 Loop A), Asp102 and Asp103 (pET28-sfGFP-SpyTag003 Loop B), or Asp173 and Gly174 (pET28-sfGFP-SpyTag003 Loop C) by Gibson assembly. pET28-sfGFP-DogTag loop insertions were derived from pET28-sfGFP by insertion of spacer-DogTag-spacer (SEQ ID NO: 11) between residues Val22 and Asn23 (pET28-sfGFP-DogTag Loop A), Asp102 and Asp103 (pET28-sfGFP-DogTag Loop B), or Asp173 and Gly174 (pET28-sfGFP-DogTag Loop C) by Gibson assembly. pGEX-2T-GST-BirA was a gift from Chris O'Callaghan, University of Oxford. pET28-MBP-sTEV is a modified TEV protease construct with the domain arrangement MBP-His₆-TEV protease-Arg₆, but with no internal TEV cleavage site between the MBP and TEV protease. The TEV protease domain contains the following solubility/stability mutations (numbers refer to the standard TEV protease numbering scheme): C19V L56V C110V C130S S135G and S219D. pET28 Affi-SnoopCatcher was created by cloning an anti-HER2 affibody on to the N-terminus of pET28 SnoopCatcher (GenBank Accession no. KU500646, Addgene plasmid ID 72322). pDEST14-Cys-DogCatcher was derived by Gibson assembly from pDEST14-DogCatcher by insertion of a cysteine between the TEV cleavage site and the DogCatcher portion.

### Protein Expression and Purification

R2Catcher, DogCatcher variants, AviTag-R2Tag-MBP, DogTag-MBP fusions, SpyTag003-MBP, SpyCatcher003-sfGFP and His₆-MBP were expressed in E. coli BL21 DE3 RIPL (Agilent). SpyCatcher003 was expressed in E. coli C41 DE3 (a gift from Anthony Watts, University of Oxford). Single colonies were inoculated into 10 mL LB containing either 100 μg/mL ampicillin (SpyCatcher003, SpyCatcher003-sfGFP, R2Catcher or DogCatcher variants) or 50 μg/mL kanamycin (His₆-MBP, SpyTag003-MBP, AviTag-R2Tag-MBP and DogTag fusions) and grown for 16 h at 37° C. with shaking at 200 rpm. For secondary culture, 1/100 dilution of the saturated overnight culture was inoculated in 1 L auto-induction LB broth plus 0.8% (v/v) glucose with appropriate antibiotic and grown at 37° C. with shaking at 200 rpm ultra-yield baffled flasks (Thomson Instrument Company) until an OD₆₀₀ of 0.5 followed by induction of overexpression with 0.42 mM IPTG at 30° ° C. with shaking at 200 rpm for 4 hours. Cells were harvested and lysed by sonication on ice in 50 mM Tris-HCl pH 8.0 containing 300 mM NaCl and 10 mM imidazole containing mixed protease inhibitors (complete mini EDTA-free protease inhibitor cocktail, Roche) and 1 mM phenylmethylsulfonyl fluoride (PMSF) and purified by Ni-NTA (Qiagen). Proteins were dialysed into PBS (137 mM NaCl, 2.7 mM KCl, 10 mM Na₂HPO₄, 1.8 mM KH₂PO₄) PH 7.5 using 3.5 kDa molecular weight cut-off dialysis tubing (Spectrum Labs). MBP-sTEV was expressed and purified as described above except without protease inhibitor cocktail tablets. Protein concentrations were determined from OD₂₈₀ using the extinction coefficient from ExPASy ProtParam.

GST-BirA was expressed in E. coli BL21 DE3 RIPL (Agilent). Single colonies were inoculated into 10 mL LB containing 100 μg/mL ampicillin and grown for 16 h at 37° C. with shaking at 200 rpm. For secondary culture, 1/100 dilution of the saturated overnight culture was inoculated in 1 L auto-induction LB broth plus 0.8% (v/v) glucose with appropriate antibiotic and grown at 37° C. with shaking at 200 rpm ultra-yield baffled flasks (Thomson Instrument Company) until an OD₆₀₀ of 0.5. Cells were induced with 0.42 mM IPTG at 30° C., with shaking at 200 rpm for 4 h. Proteins were purified using glutathione-sepharose purification as described (Fairhead and Howarth, 2015).

AviTag biotinylation with GST-BirA was performed and examined on SDS-PAGE as described (Fairhead and Howarth, 2015). Briefly, a master mix was made of 100 μM bait protein in 952 μL PBS, 5 μL 1 M MgCl₂, 20 μL 100 mM ATP, 20 μL 50 μM GST-BirA and 1.5 mM biotin. This was incubated for 1 h at 30° C. with shaking at 800 rpm. An additional 20 μL 50 μM GST-BirA was added followed by a further 1 h incubation. Finally, the bait was dialysed in PBS buffer pH 7.5 at 4° C. The extent of protein biotinylation was tested by a streptavidin gel-shift assay.

Superfolder GFP (sfGFP) variants were expressed in E. coli BL21 DE3 RIPL. Single colonies were inoculated into LB plus 50 μg/mL kanamycin and grown for 16 h at 37° C. with shaking at 200 rpm. For secondary culture, 1/100 dilution of the saturated overnight culture was inoculated into LB plus 50 μg/mL kanamycin, grown at 37° C. with shaking at 200 rpm until OD₆₀₀ reached 0.5, upon which 0.42 mM IPTG was added and the culture grown at 22° C. for 18 h. Cells were harvested and lysed by sonication on ice in 50 mM Tris-HCl PH 8.0 containing 300 mM NaCl and 10 mM imidazole containing complete mini EDTA-free protease inhibitor cocktail and 1 mM PMSF and purified by Ni-NTA (Qiagen) using standard procedures. Proteins were dialysed into PBS pH 7.5 using 3.5 kDa molecular weight cut-off dialysis tubing (Spectrum Labs). Proteins were quantified using the Pierce bicinchoninic acid (BCA) Protein assay kit (Thermo Fisher) according to the manufacturer's instructions with the modification that the proteins were incubated for 1 h at 60° C. in the assay solution before reading the absorbance.

Gre2p variants were expressed in E. coli BL21 DE3 RIPL. Single colonies were inoculated into LB plus 50 μg/mL kanamycin and grown for 16 h at 37° C. with shaking at 200 rpm. For secondary culture, 1/100 dilution of the saturated overnight culture was inoculated into LB plus 50 μg/mL kanamycin, grown at 37° C. with shaking at 200 rpm until OD₆₀₀ reached 0.5, upon which 0.42 mM IPTG was added and the culture grown for 18 h at 25° C. Cells were harvested and lysed by sonication on ice in 50 mM Tris pH 8.0 containing 300 mM NaCl and 10 mM imidazole containing mixed protease inhibitors (complete mini EDTA-free protease inhibitor cocktail, Roche) and 1 mM phenylmethylsulfonyl fluoride (PMSF) and purified by Ni-NTA (Qiagen) using standard procedures. Proteins were dialysed into 100 mM potassium phosphate pH 7.4 [formed by mixing 100 mM solutions of monobasic (KH₂PO₄) and dibasic (K₂HPO₄) potassium phosphate solutions] using 3.5 kDa molecular weight cut-off dialysis tubing (Spectrum Labs). Proteins were quantified using the Pierce BCA Protein assay kit (Thermo Fisher) according to the manufacturer's instructions

### Modeling of R2Catcher Mutations

Rosetta3 was used to model the effects of mutations on R2Catcher (Leaver-Fay et al., 2011). The crystal structure of RrgA (PDB code 2WW8) residues 734-838 with the A808P mutation was relaxed, and the pmut_scan protocol was used to calculate Rosetta Energy Units for each mutant.

### R2CatcherB WT Phage Production

Two different cell-lines were selected to identify better conditions for R2CatcherB phage production, since R2CatcherB initially displayed poorly on the phage surface. R2CatcherB phagemid was transformed into E. coli XL1-Blue (Agilent) or E. coli K12 ER2738 (Lucigen) and grown at 18, 25 or 30° C. for 16 h for phage production. Transformed cells were grown in 50 mL 2YT with 100 μg/mL ampicillin and 10 μg/mL tetracycline and 0.2% (v/v) glycerol at 37° ° C., 200 rpm until OD₆₀₀=0.5 (˜2-3 h). Cells were infected in log phase with 10¹² R408 helper phage (Agilent) and incubated at 80 rpm at 37° C. for 30 min. Expression of R2CatcherB-pIII was induced with 0.1 mM IPTG and cells were incubated for 18-20 h at 200 rpm at 18, 25 or 30° ° C. Phage were harvested using one volume of precipitation buffer [sterile, 20% (w/v) PEG8000, 2.5 M NaCl] per 4 volumes of supernatant (Keeble et al., 2017). Briefly, the supernatants were mixed with the precipitation buffer and incubated at 4° C. for 3-4 h. Phage were pelleted by centrifugation at 15,000 g for 30 min at 4° C. and the supernatant was removed. Phage pellets were resuspended in PBS (2 mL per 100 mL culture) and centrifuged at 15,000 g for 10 min at 4° C. to clear any residual cells, before the supernatant was transferred to a new tube. The mixture was precipitated again as previously, but this time resuspended in 0.25 mL PBS per 100 mL culture. Samples were centrifuged at 15,000 g for 10 min at 4° C. and phage were precipitated a third time and resuspended in a final volume of 0.25 mL PBS per 100 mL culture. Samples were stored short-term (1-2 weeks) at 4° C., or long-term at −80° ° C. with 20% glycerol (v/v) as cryoprotectant. Phage were quantified by plating serial dilutions after re-infection.

### Phage Library Generation

To create the randomised mutagenesis library, pFab5cHis R2CatcherB phagemid construct was used as a template in PCR reactions. The vector was amplified using KOD polymerase (EMD Millipore) with oligonucleotide primers (forward primer: 5′-GGATCCAGTGGTAGCGAAAACCTCTAC (SEQ ID NO: 12); reverse primer: 5′-CATGGCGCCCTGATCTCGAGG (SEQ ID NO: 13)). The insert was amplified with forward primer 5′-GACCTCGAGATCAGGGCGCCATG (SEQ ID NO: 14) and reverse primer 5′-GAAGTAGAGGTTTTCGCTACCACTGGATC (SEQ ID NO: 15) using GeneMorph II Random Mutagenesis kit (Agilent) according to the manufacturer's protocol. DpnI was added following thermal cycling, incubated at 37° C. for 1 h, and heat-inactivated at 80° C. for 20 min. The amplified fragments were separated by agarose gel electrophoresis and DNA bands for the vector and insert were purified by gel extraction (Thermo Scientific). Ligation was performed at the optimised vector:insert molar ratio of 1:3 with ˜500 ng of DNA in a total volume of 20 μL. Equal volume of 2× master mix Gibson (New England Biotech) was added to the insert-vector mixture and incubated at 50° C. for 16 h. DNA was concentrated on a spin-filter (Wizard PCR clean up kit; Promega) and 3 μL (˜700 ng) of DNA was transformed into 50 μL electrocompetent ER2738 amber stop codon suppressor cells (Lucigen) by electroporation in Bio-Rad 2 mm electroporation cuvettes in a GenePulserXcell (Bio-Rad) with a 2.5 kV voltage setting. Transformants were recovered by addition of 950 μL SOC medium at 37° ° C. for 1 h and then further grown in 50 mL 2YT media, containing 100 μg/mL ampicillin and 10 μg/mL tetracycline for 16 h at 37° C. Transformation efficiency was determined by plating serial dilutions of 1 mL rescue culture on an agar plate with 100 μg/mL ampicillin and 10 μg/mL tetracycline. Aliquots were flash-frozen and stored at −80° C. To harvest the library, 1 mL of overnight culture was added to 250 mL 2YT media with 100 μg/mL ampicillin and 10 μg/mL tetracycline and 0.2% (v/v) glycerol and grown at 37° ° C. at 200 rpm until OD₆₀₀ 0.5 (˜2-3 h). Cells were infected with 10¹² R408 helper phage (Agilent) and incubated at 80 rpm at 37° ° C. for 30 min. Expression of R2CatcherB-pIII library was induced with 0.1 mM IPTG and incubated for 18-20 h at 200 rpm at 18° C. Cells were removed by centrifugation at 15,000 g for 10 min at 4° C. and phage were purified as described above.

### Phage Selections

Biotinylated AviTag-DogTag-MBP was used as bait to react with the R2CatcherB phage library. The non-reactive bait variant (biotinylated AviTag-DogTag NA-MBP) was included in parallel selections to assess the efficiency of the panning. Reactions were carried out in PBS pH 7.5 at 25° C. with 3% (w/v) bovine serum albumin (BSA; Sigma A9418) and supplemented with 25 μM His₆-MBP (to counter-select for any DogCatcher variants that bind to MBP). In the first round of selection, 1012 phage were mixed with 0.5 μM bait and reacted for 18 h. Three subsequent selection rounds were carried out with increasing stringency (0.2 μM bait and 60 min reaction in round 2; 0.1 μM bait and 15 min reaction in round 3; 0.05 μM bait and 10 min reaction in round 4). Reaction was stopped by adding 100-fold excess bait without an AviTag (DogTag-MBP).

Phage were purified from unreacted biotinylated bait by PEG-NaCl precipitation. The pellet containing the phage-biotinylated bait adduct was resuspended in PBS pH 7.5 with 0.1% (v/v) Tween-20. 200 μL phage were mixed with 20 μL Biotin-Binder Dynabeads (Thermo Fisher Scientific) in a 96-well low bind Nunc plate that had been pre-blocked for 2 h at 25° C. with 3% (w/v) BSA in PBS pH 7.5+0.1% (v/v) Tween-20. The beads were pre-washed four times with 200 μL/well of PBS pH 7.5+0.1% (v/v) Tween-20. Phage-biotinylated bait adduct was incubated with beads in the microtiter plate for 1 h at 25° C. with shaking at 800 rpm in an Eppendorf Thermomixer. To remove weakly bound phage, beads were washed once with 150 μL glycine-HCl pH 2.2 at 25° C., then four times with 150 μL TBS (50 mM Tris-HCl+150 mM NaCl, pH 7.5) with 0.5% (v/v) Tween-20 at 25° C. Phage were eluted from beads by TEV protease digestion at 34° C. for 2 h in 50 mM Tris-HCl PH 8.0 with 0.5 mM EDTA. Eluted phage were rescued by infection of 10 mL mid-log phase (OD₆₀₀=0.5) cultures of ER2738 cells. Cells were grown at 37° C. at 80 rpm for 30 min and then transferred into 200 mL 2YT supplemented with ampicillin (100 μg/mL), tetracycline (10 μg/mL), 0.2% (v/v) glycerol and grown at 37° C. at 200 rpm for ˜2 h (until OD₆₀₀=0.5). Cultures were infected with 10¹² R408 helper phage and incubated at 80 rpm at 37° C. for 30 min. Expression of R2CatcherB-pIII was induced with 0.1 mM IPTG and cells were incubated for 18-20 h at 200 rpm at 18° C. The number of phage eluted was quantified by plating serial dilutions from 10 mL rescue culture.

### Isopeptide Bond Formation Assays

Reactions were generally carried out at 25° C. in PBS pH 7.5. Reactions were analysed by SDS-PAGE on 16% (w/v) polyacrylamide gels using the XCell SureLock system (Thermo Fisher) at 180 V. The reaction was quenched at 95° C. for 5 min after addition of 6×SDS-loading buffer [0.23 M Tris-HCl, pH 6.8, 24% (v/v) glycerol, 120 μM bromophenol blue, 0.23 M SDS] in a Bio-Rad C1000 thermal cycler. Proteins were stained using InstantBlue (Expedeon) Coomassie. Band intensities were quantified using a Gel Doc XR imager and Image Lab 5.0 software (Bio-Rad). Percentage isopeptide bond formation was calculated by dividing the intensity of the band for the covalent complex by the intensity of all the bands in the lane and multiplying by 100.

The second-order rate constant for covalent complex formation when reacting 5 μM AviTag-DogTag-MBP and 5 μM Catcher protein was determined by monitoring the reduction in the relative intensity of the band for the R2Catcher or DogCatcher, to give the change in the concentration of the unreacted Catcher variant. Time-points were analysed during the linear portion of the reaction curve. 1/[Catcher variant] was plotted against time and analysed by linear regression using Excel (Microsoft) and Origin 2015 (OriginLab Corporation), including calculation of the s.d. for the best fit. The data represent the mean±1 s.d. from triplicate measurement.

Temperature-dependence of DogTag:DogCatcher isopeptide bond formation was carried out in succinate-phosphate-glycine (SPG) buffer (12.5 mM succinic acid, 43.75 mM NaH₂PO₄, 43.75 mM glycine; pH adjusted to 7.0 using NaOH) with 2 μM of AviTag-DogTag-MBP and DogCatcher with the 15 min time-point assessed at 4, 25 or 37° C. in triplicate.

The pH-dependence of DogTag:DogCatcher isopeptide bond formation was carried out in SPG buffer with 2 μM each for AviTag-DogTag-MBP and DogCatcher with the 30 min time-point assessed at pH 4, 5, 6, 7, 8, or 9 in triplicate.

The buffer-dependence of DogTag:DogCatcher isopeptide bond formation was carried out in a range of buffers all at pH 7.5 with 5 μM AviTag-DogTag-MBP and 5 μM DogCatcher with the 5 min time point assessed. Buffers used were PBS, PBS+1 mM DTT, PBS+1 mM EDTA, PBS+1% (v/v) Triton X-100, PBS+1% (v/v) Tween-20, HBS (50 mM HEPES+150 mM NaCl), TBS (50 mM Tris-HCl+150 mM NaCl), or Tris (50 mM Tris-HCl).

Condition-dependence of SpyTag003/SpyCatcher003 was determined as follows. For the temperature-dependence assay, 100 nM SpyCatcher003-sfGFP and SpyTag003-MBP were reacted for 2 min in PBS pH 7.4 supplemented with 0.2% (w/v) BSA at 4, 25, 30 or 37° C. For the buffer-dependence assay, 100 nM SpyCatcher003-sfGFP and SpyTag003-MBP were reacted for 2 min at 25° C. in a range of buffers: PBS pH 7.4, PBS pH 7.4+1 mM EDTA (ethylenediaminetetraacetic acid), PBS pH 7.4+1% (v/v) Triton X-100, PBS pH 7.4+1% (v/v) Tween-20, HBS (20 mM HEPES pH 7.4+150 mM NaCl), or TBS (20 mM Tris-HCl PH 7.4+150 mM NaCl). Each buffer was supplemented with 0.2% (w/v) BSA. For the pH-dependence assay, 1 μM SpyCatcher003 and SpyTag003-MBP were reacted in SPG buffer at 25° C.

DogCatcher and DogTag reaction to completion was tested with 10 or 20 μM DogCatcher reacting with 10 or 20 μM HaloTag7SS-DogTag in PBS pH 7.5 for 200 min. 5 μM DogCatcher was reacted with either 5 μM HaloTag7SS-DogTag or AviTag-DogTag-MBP in PBS pH 7.5 to compare the reaction of DogTag constrained in a loop (HaloTag7SS-DogTag) or free from this constraint (AviTag-DogTag-MBP).

Reaction of loop variants for sfGFP or Gre2p was carried out in PBS pH 7.5 at 25° C. with 5 μM loop variant reacted with 5 μM DogCatcher or SpyCatcher003.

Cross-reactivity of DogCatcher (15 μM) and HaloTag7SS-DogTag (10 μM) was tested with Affi-SnoopCatcher, SnoopTagJr-AffiHer2, SpyCatcher003, SpyTag003-MBP (all at 10 μM for testing DogCatcher reactivity; with Affi-SnoopCatcher and SpyCatcher003 at 15 μM for reaction with HaloTag7SS-DogTag) in PBS pH 7.5 at 25° C. for 24 h.

### Spectroscopic Measurements

Spectra of 0.5 μM sfGFP variants were collected at 25° C. in PBS pH 7.5, using a Horiba-Yvon Fluoromax 4 with an excitation wavelength of 488 nm and fluorescence emission collected between 500 and 660 nm using a monochromator with data collected with polarizers set to the magic angle (54.7°). Absorbance spectra of 10 μM sfGFP variants were collected at 25° C. in PBS pH 7.5 using a Jasco V-550 UV/VIS Spectrophotometer. Data were collected every nm from 250 nm to 600 nm with a scanning speed of 200 nm/min, a fast response, and a bandwidth of 2.0 nm. The data represent the mean of biological triplicates.

### Gre2p Activity Assay

50 nM Gre2p variant was incubated with 1.5 mM isovaleraldehyde (Merck) and 0.25 mM reduced nicotinamide dinucleotide phosphate (NADPH) (ChemCruz) in 100 mM potassium phosphate pH 7.4 [formed by mixing 100 mM solutions of monobasic (KH₂PO₄) and dibasic (K₂HPO₄) potassium phosphate solutions]+0.1% (w/v) BSA+1 mM dithiothreitol (DTT) at 25° C. Reaction was initiated by pipetting in 100 μL of a 15 mM stock of the isovaleraldehyde in 100 mM potassium phosphate pH 7.4 to the reaction mixture and the progress was measured by the decrease in A340 measured using a Jasco V-550 UV/VIS Spectrophotometer with a medium response and 5.0 nm band width. Data were collected every second for 200 s.

### DogCatcher Dye Labelling

Dye labelling took place with tubes wrapped in foil, to minimize light exposure. Alexa Fluor 647-maleimide (Thermo Fisher) was dissolved in DMSO to 10 mg/mL. Cys-DogCatcher was dialyzed into TBS pH 7.4 and reduced for 30 min at 25° C. with 1 mM TCEP [tris(2-carboxyethyl)phosphine)]. 100 μM Cys-DogCatcher was incubated with a 3-fold molar excess of dye:protein and reacted with end-over-end rotation at 25° C. for 4 hr. After quenching the unreacted maleimide with 1 mM DTT for 30 min at 25° C., samples were centrifuged at 16,000 g for 5 min at 4° C. to remove any aggregates. Free dye was removed using Sephadex G-25 resin (Merck) and dialyzing thrice each time for at least 3 hr in PBS pH 7.4 at 4° C.

### Intracellular Calcium Measurement

HEK 293 cells were plated onto a 6-well plate at 0.8×10⁶ cells/well for 24 hr prior to transfection. Cells were transfected with 2 μg DNA for either pcDNA4/TO (empty vector), TRPC5-SYFP2, or TRPC5-DogTag-SYFP2 using jetPRIME transfection reagent (VWR). 24 hr after transfection, cells were plated onto black, clear-bottomed 96 well plates (Greiner) at 60,000 cells per well and left to adhere for 16-18 hr. For intracellular calcium recordings, media was removed and replaced with SBS containing 2 μM Fura-2 AM (Thermo Fisher) and 0.01% (v/v) pluronic acid. SBS contained (in mM): NaCl 130, KCl 5, glucose 8, HEPES 10, MgCl₂ 1.2, CaCl₂) 1.5, titrated to pH 7.4 with NaOH. Cells were then incubated for 1 hr at 37° C. After incubation, Fura-2 AM was removed and replaced with fresh SBS. Cells were incubated at 25° C. for 30 min. SBS was then replaced with recording buffer [SBS with 0.01% (v/v) pluronic acid and 0.1% (v/v) DMSO, to match compound buffer]. For experiments to determine the effect of DogCatcher labelling on TRPC5 function, cells were washed twice with SBS after Fura-2 AM incubation. SBS with or without 5 μM biotin-DogCatcher-MBP was added and cells were incubated at 25° C. for 30 min. The buffer was then replaced by recording buffer. Intracellular calcium was measured by use of a FlexStation3 (Molecular Devices), using excitation of 340 nm and 380 nm, with emission of 510 nm. Recordings were taken for 5 min at 5 s intervals. At 60 s, the agonist (−)-englerin A (PhytoLab) was added from a compound plate containing compound buffer [SBS with 0.01% (v/v) pluronic acid and (−)-englerin A] to a final concentration of 30 nM (FIG. 10A) or 10 nM (FIG. 10C).

1. A polypeptide comprising:
i) an amino acid sequence as set forth in SEQ ID NO: 1;
ii) a portion of (i) comprising an amino acid sequence as set forth in SEQ ID NO: 2;
iii) an amino acid sequence with at least 80% sequence identity to a sequence as set forth in SEQ ID NO: 1, wherein said amino acid sequence comprises a lysine at position 9, a glutamic acid at position 70 and two or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein if the amino acid sequence comprises proline at position 75, it also comprises one or more amino acid residues selected from 1)-6) and 8)-10), and wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2, wherein the amino acid sequence comprises a lysine at position 5, a glutamic acid at position 66 and one or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein if the amino acid sequence comprises proline at position 71, it also comprises one or more amino acid residues selected from 1)-5) and 7)-9), and wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

2. The polypeptide of claim 1, wherein the polypeptide comprises an amino acid sequence with at least 80% sequence identity to a sequence as set forth in SEQ ID NO: 1, wherein said amino acid sequence comprises a lysine at position 9, a glutamic acid at position 70 and three or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein the specified amino acid residues are at positions equivalent to the positions in SEQ ID NO: 1.

3. The polypeptide of claim 1, wherein the polypeptide comprises an amino acid sequence with at least 80% sequence identity to a sequence as set forth in SEQ ID NO: 1, wherein said amino acid sequence comprises a lysine at position 9, a glutamic acid at position 70, a proline at position 75 and one or more of the following:
1) isoleucine at position 69;
2) serine at position 87; and
3) arginine at position 89;
wherein the specified amino acid residues are at positions equivalent to the positions in SEQ ID NO: 1.

4. The polypeptide of claim 3, wherein the polypeptide further comprises one or more of the following:
1) glutamic acid at position 4;
2) aspartic acid at position 11;
3) threonine at position 13;
4) aspartic acid at position 47;
5) threonine at position 59; and
6) aspartic acid at position 92;
wherein the specified amino acid residues are at positions equivalent to the positions in SEQ ID NO: 1.

5. A polypeptide comprising:
i) an amino acid sequence as set forth in SEQ ID NO: 1;
ii) a portion of (i) comprising an amino acid sequence as set forth in SEQ ID NO: 2;
iii) an amino acid sequence with at least 80% sequence identity to a sequence as set forth in SEQ ID NO: 1, wherein said amino acid sequence comprises a lysine at position 9, a glutamic acid at position 70, one or more of the following:
1) glutamic acid at position 4;
2) aspartic acid at position 11;
3) threonine at position 13;
4) aspartic acid at position 47;
5) threonine at position 59;
6) proline at position 75; and
7) aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 2, wherein the amino acid sequence comprises a lysine at position 5, a glutamic acid at position 66, one or more of the following:
1) aspartic acid at position 7;
2) threonine at position 9;
3) aspartic acid at position 43;
4) threonine at position 55;
5) proline at position 71; and
6) aspartic acid at position 88;
and one or more of the following:
1) isoleucine at position 65;
2) serine at position 83; and
3) arginine at position 85;
wherein the specified amino acid residues are at positions equivalent to the positions in SEQ ID NO: 2,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

6. The polypeptide of claim 5, wherein the polypeptide comprises an amino acid sequence with at least 80% sequence identity to a sequence as set forth in SEQ ID NO: 1, wherein said amino acid sequence comprises a lysine at position 9, a glutamic acid at position 70, two or more of the following:
1) glutamic acid at position 4;
2) aspartic acid at position 11;
3) threonine at position 13;
4) aspartic acid at position 47;
5) threonine at position 59;
6) proline at position 75; and
7) aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1.

7. The polypeptide according to any one of claims 1 to 6, wherein the polypeptide comprises an amino acid sequence with at least 80% sequence identity to a sequence as set forth in SEQ ID NO: 1, wherein said amino acid sequence comprises a lysine at position 9, a glutamic acid at position 70, a proline at position 75, one or more of the following:
1) glutamic acid at position 4;
2) aspartic acid at position 11;
3) threonine at position 13;
4) aspartic acid at position 47;
5) threonine at position 59; and
6) aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1,
and wherein said polypeptide is capable of spontaneously forming an isopeptide bond with a peptide comprising an amino acid sequence as set forth in SEQ ID NO: 3, wherein said isopeptide bond forms between the asparagine residue at position 17 of SEQ ID NO: 3 and the lysine residue at position 9 of SEQ ID NO: 1 or position 5 of SEQ ID NO: 2.

8. The polypeptide of any one of claims 1 to 7, wherein the polypeptide comprises an amino acid sequence with at least 80% sequence identity to a sequence as set forth in SEQ ID NO: 1, wherein said amino acid sequence comprises a lysine at position 9, a glutamic acid at position 70 and all of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein the specified amino acid residues are at positions equivalent to the positions in SEQ ID NO: 1.

9. The polypeptide of any one or claims 1 to 8, wherein the polypeptide is conjugated to a nucleic acid molecule, protein, peptide, small-molecule organic compound, fluorophore, metal-ligand complex, polysaccharide, nanoparticle, 2D monolayer (e.g. graphene), lipid, nanotube, polymer, cell, virus, virus-like particle, viral vector or a combination thereof.

10. A polypeptide comprising:
i) an amino acid sequence as set forth in SEQ ID NO: 18, wherein X at position 70 is not glutamic acid or aspartic acid, optionally wherein X at position 70 is selected from alanine, glycine, serine, asparagine, or threonine;
ii) a portion of (i) comprising an amino acid sequence as set forth in SEQ ID NO: 19, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 18, wherein X at position 70 is not glutamic acid or aspartic acid, optionally wherein X at position 70 is selected from alanine, glycine, serine, asparagine, or threonine, and wherein the amino acid sequence comprises one or more of the following:
1) glutamic acid at position 4;
2) aspartic acid at position 11;
3) threonine at position 13;
4) aspartic acid at position 47;
5) threonine at position 59;
6) isoleucine at position 69;
7) proline at position 75;
8) serine at position 87;
9) arginine at position 89; and
10) aspartic acid at position 92;
wherein if the amino acid sequence comprises proline at position 75, it also comprises one or more amino acid residues selected from 1)-6) and 8)-10), and wherein the specified amino acid residues are at positions equivalent to the positions in SEQ ID NO: 18; or
iv) a portion of (iii) comprising an amino acid sequence with at least 80% sequence identity to a sequence as set forth in SEQ ID NO: 19, wherein X at position 66 is not glutamic acid or aspartic acid, optionally wherein X at position 66 is selected from alanine, glycine, serine, asparagine, or threonine and wherein the amino acid sequence comprises one or more of the following:
1) aspartic acid at position 7;
2) threonine at position 9;
3) aspartic acid at position 43;
4) threonine at position 55;
5) isoleucine at position 65;
6) proline at position 71;
7) serine at position 83;
8) arginine at position 85; and
9) aspartic acid at position 88;
wherein if the amino acid sequence comprises proline at position 71, it also comprises one or more amino acid residues selected from 1)-5) and 7)-9), and wherein the specified amino acid residues are at positions equivalent to the positions in SEQ ID NO: 19,
and wherein the polypeptide binds selectively and reversibly to a peptide comprising an amino acid sequence as set forth in SEQ ID NO: 3.

11. A polypeptide comprising:
i) an amino acid sequence as set forth in SEQ ID NO: 18, wherein X at position 70 is not glutamic acid or aspartic acid, optionally wherein X at position 70 is selected from alanine, glycine, serine, asparagine, or threonine;
ii) a portion of (i) comprising an amino acid sequence as set forth in SEQ ID NO: 19, wherein X at position 66 is not glutamic acid or aspartic acid, optionally wherein X at position 66 is selected from alanine, glycine, serine, asparagine, or threonine;
iii) an amino acid sequence with at least 80% sequence identity to a sequence as set forth in SEQ ID NO: 18, wherein X at position 70 is not glutamic acid or aspartic acid, optionally wherein X at position 70 is selected from alanine, glycine, serine, asparagine, or threonine, and wherein the amino acid sequence comprises one or more of the following:
1) glutamic acid at position 4;
2) aspartic acid at position 11;
3) threonine at position 13;
4) aspartic acid at position 47;
5) threonine at position 59;
6) proline at position 75; and
7) aspartic acid at position 92;
and one or more of the following:
1) isoleucine at position 69;
2) serine at position 87; and
3) arginine at position 89;
wherein the specified amino acid residues are at positions equivalent to the positions in SEQ ID NO: 1; or
iv) a portion of (iii) comprising an amino acid sequence with at least 80% sequence identity to a sequence as set forth in SEQ ID NO: 19, wherein X at position 66 is not glutamic acid or aspartic acid, optionally wherein X at position 66 is selected from alanine, glycine, serine, asparagine, or threonine and wherein the amino acid sequence comprises one or more of the following:
1) aspartic acid at position 7;
2) threonine at position 9;
3) aspartic acid at position 43;
4) threonine at position 55;
5) proline at position 71; and
6) aspartic acid at position 88;
and one or more of the following:
1) isoleucine at position 65;
2) serine at position 83; and
3) arginine at position 85;
wherein the specified amino acid residues are at positions equivalent to the positions in SEQ ID NO: 19,
and wherein the polypeptide binds selectively and reversibly to a peptide comprising an amino acid sequence as set forth in SEQ ID NO: 3.

12. The polypeptide of claim 10 or 11, wherein the polypeptide comprises an amino acid sequence with at least 80% sequence identity to a sequence as set forth in SEQ ID NO: 18 or 19 and wherein the amino acid sequence comprises lysine at a position equivalent to position 9 in SEQ ID NO: 18 or position 5 in SEQ ID NO: 19.

13. The polypeptide of any one of claims 10 to 12, wherein the polypeptide comprises an additional N-terminal or C-terminal sequence comprising a cysteine residue.

14. The polypeptide of any one of claims 10 to 12, wherein the polypeptide comprises an amino acid sequence with at least 80% sequence identity to a sequence as set forth in SEQ ID NO: 18 or 19, wherein the polypeptide comprises a cysteine residue.

15. The polypeptide of claim 14, wherein the cysteine residue is at a position equivalent to position 31 or 41 in SEQ ID NO: 18 or a position equivalent to position 27 or 37 in SEQ ID NO: 19.

16. The polypeptide of any one of claims 1 to 15, wherein the polypeptide is immobilised on a solid substrate.

17. The polypeptide of any one of claims 1 to 16, wherein the polypeptide is immobilised on a solid substrate via a covalent bond.

18. The polypeptide of any one of claims 10 to 15, wherein the polypeptide is immobilised on a solid substrate via a covalent bond between a cysteine residue and the solid substrate.

19. A recombinant or synthetic polypeptide comprising a peptide or polypeptide linked to a polypeptide as defined in any one of claims 1 to 18.

20. A nucleic acid molecule comprising a nucleotide sequence which encodes the polypeptide of any one of claims 1 to 8 or 10 to 18 or the recombinant polypeptide of claim 19.

21. A vector comprising the nucleic acid molecule of claim 20.

22. A cell comprising the nucleic acid molecule of claim 20 or the vector of claim 21.

23. A process for producing or expressing the polypeptide of any one of claims 1 to 8 or 10 to 18 or the recombinant polypeptide of claim 19 comprising the steps of:
a) transforming or transfecting a host cell with a vector as defined in claim 21;
b) culturing the host cell under conditions which allow the expression of the polypeptide; and optionally
c) isolating the polypeptide.

24. Use of a polypeptide as defined in any one of claim 1 to 9 or 16 to conjugate two molecules or components via an isopeptide bond,
wherein said molecules or components conjugated via an isopeptide bond comprise:
a) a first molecule or component comprising a polypeptide of any one of claim 1 to 9 or 16; and
b) a second molecule or component comprising a peptide selected from:
(i) a peptide comprising an amino acid sequence as set forth in any one of SEQ ID NOs: 3-5 or 17; and
(ii) a peptide comprising an amino acid sequence with at least 80% sequence identity to a sequence as set forth in any one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11,
and wherein said peptide is capable of spontaneously forming an isopeptide bond with a polypeptide comprising an amino acid sequence as set forth in SEQ ID NO: 1, wherein said isopeptide bond forms between the asparagine residue at position 17 of SEQ ID NO: 3, 4, 5 or 17 and the lysine residue at position 9 of SEQ ID NO: 1.

25. The use of claim 24, wherein the second molecule or component comprises the peptide at an internal site.

26. The use of claim 24 or 25, wherein the second molecule or component is a protein and wherein said protein comprises the peptide within a loop.

27. A process for conjugating two molecules or components via an isopeptide bond comprising:
a) providing a first molecule or component comprising a polypeptide of any one of claim 1 to 9 or 16;
b) providing a second molecule or component comprising a peptide selected from:
(i) a peptide comprising an amino acid sequence as set forth in any one of SEQ ID NOs: 3-5 or 17; and
(ii) a peptide comprising an amino acid sequence with at least 80% sequence identity to a sequence as set forth in any one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11,
wherein said peptide is capable of spontaneously forming an isopeptide bond with a polypeptide comprising an amino acid sequence as set forth in SEQ ID NO: 1, wherein said isopeptide bond forms between the asparagine residue at position 17 of SEQ ID NO: 3, 4, 5 or 17 and the lysine residue at position 9 of SEQ ID NO: 1; and
c) contacting said first and second molecules or components under conditions that enable the spontaneous formation of an isopeptide bond between the polypeptide and peptide, thereby conjugating said first molecule or component to said second molecule or component via an isopeptide bond to form a complex.

28. The process of claim 27, wherein the second molecule or component comprises the peptide at an internal site.

29. The process of claim 27 or 28, wherein the second molecule or component is a protein and wherein said protein comprises the peptide within a loop.

30. A kit, preferably for use in the use of any one of claims 24 to 26 or the process of any one of claims 27 to 29, wherein said kit comprises:
(a) a polypeptide of any one of claim 1 to 9 or 16, optionally conjugated or fused to a molecule or component; and
(b) a peptide, optionally conjugated or fused to a molecule or component, selected from:
(i) a peptide comprising an amino acid sequence as set forth in any one of SEQ ID NOs: 3-5 or 17; and
(ii) a peptide comprising an amino acid sequence with at least 80% sequence identity to a sequence as set forth in any one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11,
wherein said peptide is capable of spontaneously forming an isopeptide bond with a polypeptide comprising an amino acid sequence as set forth in SEQ ID NO: 1, wherein said isopeptide bond forms between the asparagine residue at position 17 of SEQ ID NO: 3, 4, 5 or 17 and the lysine residue at position 9 of SEQ ID NO: 1, optionally conjugated or fused to a molecule or component; and/or
(c) a nucleic acid molecule, particularly a vector, encoding a polypeptide as defined in (a); and/or
(d) a nucleic acid molecule, particularly a vector, encoding a peptide as defined in (b).

31. The use of any one of claims 24 to 26, process of any one of claims 27 to 29 or kit of claim 30, wherein the peptide is selected from:
(i) a peptide comprising an amino acid sequence as set forth in SEQ ID NO: 3; and
(ii) a peptide comprising an amino acid sequence with at least 80% sequence identity to a sequence as set forth in SEQ ID NO: 3, wherein the amino acid sequence comprises a threonine residue at position 5, an aspartic acid residue at position 10 and a glycine residue at position 11 and an asparagine residue at position 17,
wherein the specified amino acid residues are at positions equivalent to the positions in SEQ ID NO: 3.

32. A process for purifying or isolating a molecule or component comprising a peptide having an amino acid sequence with at least 80% sequence identity to a sequence as set forth in one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11, said process comprising:
a) providing a solid substrate on which a polypeptide of any one of claims 10 to 15 is immobilised;
b) providing a sample comprising said molecule or component;
c) contacting the solid substrate of a) with the sample of b) under conditions that enable said peptide to selectively bind to said polypeptide, thereby forming a non-covalent complex between said polypeptide immobilised on the solid substrate and molecule or component comprising said peptide;
d) washing the solid substrate with a buffer;
e) separating the molecule or component comprising the peptide from the polypeptide immobilised on the solid substrate.

33. Use of a polypeptide of any one of claims 10 to 18 to purify or isolate a molecule or component comprising a peptide having an amino acid sequence with at least 80% sequence identity to a sequence as set forth in one of SEQ ID NOs: 3-5 or 17, wherein the amino acid sequence comprises an asparagine residue at position 17 and optionally comprises a threonine residue at position 5, an aspartic acid residue at position 10 and a glycine residue at position 11.

34. An apparatus for use in the process of claim 32 or use of claim 33 comprising a solid substrate on which a polypeptide of any one of claims 10 to 15 is immobilised.

35. A kit for use in preparing a solid substrate on which a polypeptide of any one of claims 10 to 15 is immobilised, comprising:
a) a polypeptide of any one of claims 10 to 15; and
b) means for immobilising the polypeptide of a) on a solid substrate.

36. The use, process or kit of any preceding claim, wherein the peptide comprises an amino acid sequence as set forth in SEQ ID NO: 3.


SEQUENCE LISTING (24 sequences)

SEQ ID NO: 1 - protein, 104 aa, Artificial Sequence
  DogCatcher
KLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 2 - protein, 95 aa, Artificial Sequence
  DogCatcher Truncation
IEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVT

SEQ ID NO: 3 - protein, 23 aa, Artificial Sequence
  DogTag
DIPATYEFTDGKHYITNEPIPPK

SEQ ID NO: 4 - protein, 19 aa, Artificial Sequence
  RrgATag
DIPATYEFTNDKHYITNEP

SEQ ID NO: 5 - protein, 23 aa, Artificial Sequence
  RrgATag2
DIPATYEFTNGKHYITNEPIPPK

SEQ ID NO: 6 - protein, 104 aa, Artificial Sequence
  RrgACatcher
KLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 7 - DNA, 312 bp, Artificial Sequence
  DogCatcher DNA
aaactgggcgagattgaatttattaaagtggacaaaaccgataaaaagccgctgcgtggtgccgtgtttagcctgcagaaacagcatcccgactatcccgatatctatggcgcgattgatcagaatgggacctatcaagatgtgcgtaccggcgaagatggtaaactgacctttacgaatctgagcgatggcaaatatcgcctgattgaaaatagcgaacccccgggctataaaccggtgcagaataagccgattgtgagctttcgtattgtggatggcgaagtgcgtgatgtgaccagtattgtgccgcag

SEQ ID NO: 8 - protein, 104 aa, Artificial Sequence
  RrgACatcherB
KLGEIEFIKVDKTDKKPLRGAVFSLQKQHPDYPDIYGAIDQNGTYQDVRTGEDGKLTFTNLSDGKYRLFENSEPPGYKPVQNKPIVAFQIVDGEVRDVTSIVPQ

SEQ ID NO: 9 - protein, 104 aa, Artificial Sequence
  RrgACatcher with reactivity modifications
KLGDIEFIKVNKNDKKPLRGAVFSLQKQHPDYPDIYGAIDQNGTYQNVRTGEDGKLTFKNLSDGKYRLIENSEPAGYKPVQNKPIVSFRIVNGEVRDVTSIVPQ

SEQ ID NO: 10 - protein, 26 aa, Artificial Sequence
  Spacer-SpyTag003-Spacer
GGGGSRGVPHIVMVDAYKRYKGGGGS

SEQ ID NO: 11 - protein, 33 aa, Artificial Sequence
  Spacer-DogTag-Spacer
GGGGSDIPATYEFTDGKHYITNEPIPPKGGGGS

SEQ ID NO: 12 - DNA, 27 bp, Artificial sequence
  Primer
ggatccagtggtagcgaaaacctctac

SEQ ID NO: 13 - DNA, 21 bp, Artificial sequence
  Primer
catggcgccctgatctcgagg

SEQ ID NO: 14 - DNA, 23 bp, Artificial sequence
  Primer
gacctcgagatcagggcgccatg

SEQ ID NO: 15 - DNA, 29 bp, Artificial sequence
  Primer
gaagtagaggttttcgctaccactggatc

SEQ ID NO: 16 - protein, 9 aa, Artificial Sequence
  Linker
GTSGSGGSG

SEQ ID NO: 17 - protein, 23 aa, Artificial Sequence
  R2Tag
DIPAGYEFTNDKHYITNEPIPPK

SEQ ID NO: 18 - protein, 104 aa, Artificial Sequence
  Affinity purification polypeptide
  MISC_FEATURE (70)..(70): Xaa is not glutamic acid or aspartic acid
KLGEIEFIKVDKTDKKPLRGAVFSLQKQHPDYPDIYGAIDQNGTYQDVRTGEDGKLTFTNLSDGKYRLIXNSEPPGYKPVQNKPIVSFRIVDGEVRDVTSIVPQ

SEQ ID NO: 19 - protein, 95 aa, Artificial Sequence
  Truncated Affinity purification polypeptide
  MISC_FEATURE (66)..(66): Xaa is not glutamic acid or aspartic acid
IEFIKVDKTDKKPLRGAVFSLQKQHPDYPDIYGAIDQNGTYQDVRTGEDGKLTFTNLSDGKYRLIXNSEPPGYKPVQNKPIVSFRIVDGEVRDVT

SEQ ID NO: 20 - protein, 131 aa, Artificial sequence
  Affinity purification polypeptide extension
  MISC_FEATURE (97)..(97): Xaa is not glutamic acid or aspartic acid
MSYYHHHHHHDYDIPTTENLYFQGGCGKLGEIEFIKVDKTDKKPLRGAVFSLQKQHPDYPDIYGAIDQNGTYQDVRTGEDGKLTFTNLSDGKYRLIXNSEPPGYKPVQNKPIVSFRIVDGEVRDVTSIVPQ

SEQ ID NO: 21 - protein, 104 aa, Artificial sequence
  Affinity purification polypeptide D31C
  MISC_FEATURE (70)..(70): Xaa is not glutamic acid or aspartic acid
KLGEIEFIKVDKTDKKPLRGAVFSLQKQHPCYPDIYGAIDQNGTYQDVRTGEDGKLTFTNLSDGKYRLIXNSEPPGYKPVQNKPIVSFRIVDGEVRDVTSIVPQ

SEQ ID NO: 22 - protein, 104 aa, Artificial sequence
  Affinity purification polypeptide Q41C
  MISC_FEATURE (70)..(70): Xaa is not glutamic acid or aspartic acid
KLGEIEFIKVDKTDKKPLRGAVFSLQKQHPDYPDIYGAIDCNGTYQDVRTGEDGKLTFTNLSDGKYRLIXNSEPPGYKPVQNKPIVSFRIVDGEVRDVTSIVPQ

SEQ ID NO: 23 - protein, 95 aa, Artificial sequence
  Truncated Affinity purification polypeptide D27C
  MISC_FEATURE (66)..(66): Xaa is not glutamic acid or aspartic acid
IEFIKVDKTDKKPLRGAVFSLQKQHPCYPDIYGAIDQNGTYQDVRTGEDGKLTFTNLSDGKYRLIXNSEPPGYKPVQNKPIVSFRIVDGEVRDVT

SEQ ID NO: 24 - protein, 95 aa, Artificial sequence
  Truncated Affinity purification polypeptide Q37C
  MISC_FEATURE (66)..(66): Xaa is not glutamic acid or aspartic acid
IEFIKVDKTDKKPLRGAVFSLQKQHPDYPDIYGAIDCNGTYQDVRTGEDGKLTFTNLSDGKYRLIXNSEPPGYKPVQNKPIVSFRIVDGEVRDVT